(19) United States
(12) Patent Application Publication
(10) Pub. No.: US 2011/0280940 A1
(43) Pub. Date: Nov. 17, 2011

(54) Di-Vanilloyl And Tri-Vanilloyl Derivatives For Use In Anti-Cancer Therapy

(76) Inventors: Robert Kiss, Sint-Pieters-Leeuw (BE); Jacques Dubois, Villers la Ville (BE); Jean Neve, Bruxelles (BE); Delphine Lamoral-Theys, Bruxelles (BE); Francois Dufrasne, Bruxelles (BE); Laurent Pottier, Bruxelles (BE)

(21) Appl. No.: 12/998,410

(22) PCT Filed: Oct. 13, 2009
(86) PCT No.: PCT/EP2009/063369

§ 371 Date: Apr. 15, 2011

Related U.S. Application Data
(60) Provisional application No. 61/106,251, filed on Oct. 17, 2008.
(60) Provisional application No. 61/166,439, filed on Apr. 3, 2009.

(30) Foreign Application Priority Data
Oct. 17, 2008 (EP) 08166852.7
Mar. 4, 2009 (EP) 09157289.1

(51) Int. Cl. A61K 31/235; A61K 9/36; C12Q 1/48; A61P 25/00; C07C 69/84; A61P 35/00; A61P 25/28; A61P 25/16; C07C 69/92; C07C 69/76

(52) U.S. Cl. 424/475; 560/67; 514/533; 435/15; 560/64; 560/72

(57) ABSTRACT

The invention relates to the medical field, more precisely in the field of anti-cancer treatment and treatment of Alzheimer's disease, Parkinson's disease or Pick's disease or for ameliorating symptoms of Down syndrome, providing newly synthesised multi-vanilloyl derivative compounds and their use in the treatment of said disorders.

### FIELD OF THE INVENTION

The invention lies in the medical field, more precisely in the field of new therapeutic compounds, more particularly for use in anti-cancer treatment or in treatment of Down syndrome and to sickle cell anemia disease using newly synthesized divanilloyl derivatives.

### BACKGROUND OF THE INVENTION

The search for novel anti-cancer drugs is a never ending story, since cancer is becoming a more and more important cause of death amongst humans. More than 80% of all anti-cancer drugs are directed towards the apoptosis pathway of tumor cells and are cytotoxic upon activating said pathway.

A large number of cancer cells such as glioblastomas (brain cancers), brain metastases, melanomas, pancreatic cancers, lung cancers of the NSCLC-type, refractory prostate cancers (HRPC), breast cancers such as triple negatives and other types are naturally resistant to apoptosis and cannot be treated by the many known drugs and chemotherapeutics. The present invention therefore investigated the potential of new compounds, to have a cytotoxic and/or a cytostatic effect on apoptosis resistant tumor cells or cancer cells.

There is a continuous need in the art for improving the efficacy of antiproliferative treatments in humans by providing suitable combinations of new drugs with conventional antineoplastic agents.

### SUMMARY OF THE INVENTION

The invention provides a solution to the above stated problem by providing new compounds based on a common structure comprising vanillic acid groups.

Previously, 3 new isomeric divanilloylquinic acids respectively named Burkinabin A, B and C from the root bark of Fagara zanthoxyloides, an African tree growing in Burkina Faso were isolated. Burkinabins are associated with erythrocyte antisickling activity, knowing that sickle cell disease seems to occur through ion channels impairment followed by actin cytoskeleton disorganization. In sickle cell disease, it is thus an impairment of ion channels that impairs erythrocyte biology. Since actin cytoskeleton is also a key player in cell division (cytokinesis) and cell motility, the inventors hypothesized that impairment of ion channels implicated in cancer cell division and motility (migration) could mimic “sickle cell installment” in cancer cells, a feature that in turn could impair cancer cell division and migration.

The inventors therefore set up a program of full chemical synthesis in order to obtain simplified burkinabins in a limited number of chemical steps (<6) and thus designed and synthesized polyvanilloyl (e.g. di- and tri-vanilloyl) derivatives. These derivatives are relatively easy and cheap to synthesize, which is already an advantage over a lot of known drugs or chemotherapeutics.

The compounds of the invention were subsequently evaluated for their 1) anti-proliferative effect (by means of the colorimetric MTT assay), 2) pro-autophagic and pro-apoptotic effect (by means of flow cytometry analyses), and 3) anti-migratory effect (by means of quantitative videomicroscopy), in the following human cancer cell-lines: a) U373, T98G and Hs683 glioblastoma cells, b) VM21 and VM48 melanoma cells, c) PC-3 prostate cancer cells, d) MCF-7 breast cancer cells, e) LoVo colon cancer cells, f) OE21 oesophageal cancer cells, and g) A549 NSCLC cancer cells, as well as in human WS1 and WI38 normal fibroblasts (i.e. non-cancer cells). The inventors have unexpectedly found that the backbone of the simplified burkinabin vanilloyd derivatives, i.e. the compounds of the invention, appear to have important anti-cancer effects at a given concentration, while not impairing normal cell biology of non-tumor or non-cancer cells at said concentration.

In addition, the inventors could establish that the compounds of the invention act through a kinase-related, but certainly non-apoptosis-related mechanism, making them good candidates as anti-cancer drugs for treating apoptosis-resistant tumor or cancer cells and for overcoming problems linked to the known anti-cancer drugs.

The invention relates to methods and compounds for treating proliferative disorders (such as cancers). In particular, the invention provides di- and tri-vanilloyl derivative compounds and the use thereof for treating proliferative disorders (such as cancers). In addition, the invention also provides for methods of treatment of oxidative disorders, inflammatory disorders, Alzheimer's disease, Parkinson's disease, Pick's disease or for ameliorating symptoms of Down syndrome and the invention provides di- and tri-vanilloyl derivative compounds and the use thereof for treating oxidative disorders, inflammatory disorders, Alzheimer's disease, Parkinson's disease, Pick's disease or for ameliorating symptoms of Down syndrome.

The compounds of the invention have the general formula (I):

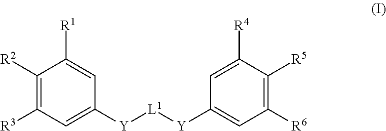

wherein R¹⁻⁶ can be each independently of each other ═H, OH, C₁₋₈ alkoxyalkylene, OMe, Ac, OAc, C₁₋₈ alkyl, NO₂ or a halogen such as F or Cl, and wherein two contiguous substituents among R¹⁻³ can be together a dioxole;
wherein Y is selected from the group comprising COO, tetrazole, OCO, OCOO, CONR¹⁰, NR¹⁰CO, OCONR¹⁰, NR¹⁰COO, NR¹⁰CONR¹⁰, COCH₂CO, COCH₂CH₂, CH₂CH₂CO, CH₂COCH₂, COOCH₂, CONHCH₂, CON—C₁₋₆alkylCH₂, CONHCO, CON—C₁₋₆alkylCO, CH₂NHCH₂, CH₂N—C₁₋₆alkylCH₂, CH₂OCO, CH₂NHCO, CH₂N(C₁₋₆alkyl)CO, CH₂OCH₂, CH₂SCH₂, SO₂OCH₂, SO₂NHCH₂, and SO₂N—C₁₋₆alkylCH₂, wherein R¹⁰═H, C₁₋₄ Alkyl;
wherein L¹=C₁₋₈alkylene, preferably C₅₋₁₀ alkylene; or
wherein L¹=(CH₂)ₙ, wherein n is an integer selected from 2-10; or
wherein

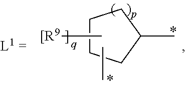

or
wherein

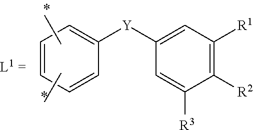

(* The asterisk is used herein to indicate the point at which a mono- or bivalent radical depicted is connected to the structure to which it relates and of which the radical forms part),
wherein R⁹ is selected from the group comprising OH, CO₂H, or NH₂ and q is an integer selected from 0, 1, 2, or 3, each group being optionally substituted with one, two or three substituents each independently selected form the group comprising C₁₋₆alkyl, CO₂H, vanillic acid, amine, and C₁₋₆alkyloxycarbonyl, wherein p is an integer selected from 0, 1, 2, or 3; or stereoisomeric forms thereof and the pharmaceutically acceptable addition salts, hydrates or solvates thereof.

These compounds have been shown by the inventors to be useable for treating proliferative disorders, oxidative disorders, inflammatory disorders, Alzheimer's disease, Parkinson's disease, Pick's disease or for ameliorating symptoms of Down syndrome.

In preferred embodiments, the Y is COO or COOCH₂.

In preferred embodiments, R¹ and R⁴ are each independently hydrogen, and R², R³, R⁵ and R⁶ are each independently hydrogen, hydroxyl, or C₁₋₆alkoxy.

In further preferred embodiments, R¹ and R⁴ are each independently hydrogen, and R², R³, R⁵ and R⁶ are each independently hydroxyl or C₁₋₆alkoxy.

In further preferred embodiments, R¹ and R⁴ are each independently hydrogen, R² and R⁵ are each independently C₁₋₆alkoxy and R³ and R⁶ are each independently hydroxyl.

In further preferred embodiments, R¹ and R⁴ are each independently hydrogen, R² and R⁵ are each independently methoxy and R³ and R⁶ are each independently hydroxyl.

In a further preferred embodiment, Y═COOCH₂,

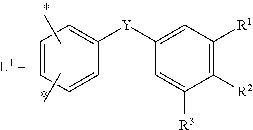

(* The asterisk is used herein to indicate the point at which a mono- or bivalent radical depicted is connected to the structure to which it relates and of which the radical forms part), R¹ and R⁴═OMe, F or Cl, R² and R⁵═OH, and R³ and R⁶═H (DLT-95, DLT-95-F and DLT-95-Cl),

In a further embodiment of the invention, the compounds of the invention have the general formula (II):

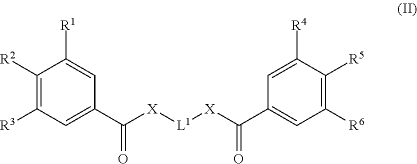

wherein X is selected from the group comprising O, O—C₁₋₆alkyl, NH, and N—C₁₋₆alkyl;
wherein each R¹, R², R³, R⁴, R⁵, R⁶ is independently selected from the group comprising H, OH, C₁₋₈alkoxyC₁₋₆alkyl, C₁₋₆alkoxy, and halogen such as e.g. F and Cl;
wherein L¹ is a group selected from C₁₋₈alkylene,

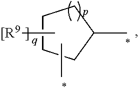

or

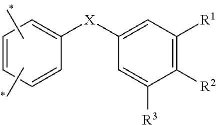

(* The asterisk is used herein to indicate the point at which a mono- or bivalent radical depicted is connected to the structure to which it relates and of which the radical forms part);
each group being optionally substituted with one, two or three substituents each independently selected form the group comprising C₁₋₆alkyl, CO₂H, vanillic acid, amine, and C₁₋₆alkyloxycarbonyl, wherein p is an integer selected from 0, 1, 2, or 3;
wherein R⁹ is selected from the group comprising OH, CO₂H, and NH₂ and q is an integer selected from 0, 1, 2, or 3;
or stereoisomeric forms thereof, and the pharmaceutically acceptable addition salts, hydrates or solvates thereof.

These compounds have been shown by the inventors to be useable for treating proliferative disorders, oxidative disorders, inflammatory disorders, Alzheimer's disease, Parkinson's disease, Pick's disease or for ameliorating symptoms of Down syndrome.

In preferred embodiments, the X is O.

In alternatively preferred embodiments, the X is NH.

In preferred embodiments, R¹ and R⁴ are each independently hydrogen, and R², R³, R⁵ and R⁶ are each independently hydrogen, hydroxyl, or C₁₋₆alkoxy.

In further preferred embodiments, R¹ and R⁴ are each independently hydrogen, and R², R³, R⁵ and R⁶ are each independently hydroxyl or C₁₋₆alkoxy.

In further preferred embodiments, R¹ and R⁴ are each independently hydrogen, R² and R⁵ are each independently C₁₋₆alkoxy and R³ and R⁶ are each independently hydroxyl.

In further preferred embodiments, R¹ and R⁴ are each independently hydrogen, R² and R⁵ are each independently methoxy and R³ and R⁶ are each independently hydroxyl.

In a further preferred embodiment,

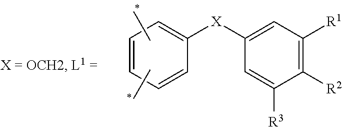

(* The asterisk is used herein to indicate the point at which a mono- or bivalent radical depicted is connected to the structure to which it relates and of which the radical forms part), R¹ and R⁴═OMe, F or Cl, R² and R⁵═OH, and R³ and R⁶═H (DLT-95, DLT-95-F and DLT-95-Cl).

In further preferred embodiments, the compound of the invention is selected from the group comprising the compounds of table 1.

In addition, the invention provides compounds of the formula (III)

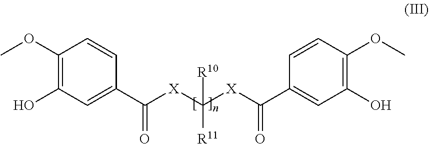

wherein X is selected from the group comprising O, O—C₁₋₆alkyl, NH, and N—C₁₋₆alkyl;
wherein n is an integer selected from 1, 2, 3, 4, 5, 6, 7, or 8.
wherein R¹⁰ and R¹¹ are, each independently selected from the group comprising H, CO₂H, C₁₋₆alkyl, amine and vanillic acid.

In further preferred embodiments X is oxygen and n is 2.

In a further preferred embodiment, X is NH and N is 2.

In a more preferred embodiment, the compounds of the invention are selected from the group comprising Ethane-1,2-diyl bis-(4-hydroxy-3-methoxybenzoate), Propane-1,3-diyl bis-(4-hydroxy-3-methoxybenzoate), Butane-1,4-diyl bis-(4-hydroxy-3-methoxybenzoate), Pentane-1,5-diyl bis-(4-hydroxy-3-methoxybenzoate), Hexane-1,6-diyl bis-(4-hydroxy-3-methoxybenzoate).

Additionally, the invention provides compounds of the formula (IV)

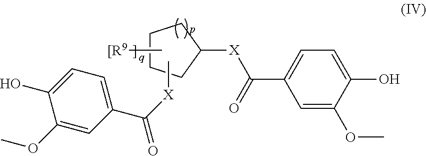

wherein X is selected from the group comprising O, O—C₁₋₆alkyl, NH, and N—C₁₋₆alkyl;
wherein p is an integer selected from 0, 1, 2, or 3; and
wherein R⁹ is selected from the group comprising OH, CO₂H, NH₂ and q is an integer selected from 0, 1, 2, or 3.

In a preferred embodiment, X is oxygen, p is 2 and q is 0.

In a further preferred embodiment, X is NH, p is 2 and q is 0.

In further preferred embodiments, the compound of the invention is selected from the group comprising trans-cyclohexane-1,2-diyl bis-(4-hydroxy-3-methoxybenzoate); cis-cyclohexane-1,2-diyl bis-(4-hydroxy-3-methoxybenzoate); racemic cyclohexane-1,3-diyl bis-(4-hydroxy-3-methoxybenzoate); cis-cyclohexane-1,3-diyl bis-(4-hydroxy-3-methoxybenzoate); trans-cyclohexane-1,3-diyl bis-(4-hydroxy-3-methoxybenzoate); cis-cyclohexane-1,4-diyl bis-(4-hydroxy-3-methoxybenzoate); trans-cyclohexane-1,4-diyl bis-(4-hydroxy-3-methoxybenzoate); racemic cyclohexane-1,4-diyl bis-(4-hydroxy-3-methoxybenzoate).

In an even more preferred embodiment, the compound of the invention is trans-cyclohexane-1,2-diyl bis-(4-hydroxy-3-methoxybenzoate).

In a further embodiment, the compounds of the invention is selected from the group comprising: [2-[(4-hydroxy-3-methoxy-benzoyl)oxymethyl]phenyl]methyl 4-hydroxy-3-methoxy-benzoate (DLT 24), 6-(3,4-dimethoxybenzoyl)oxyhexyl 3,4-dimethoxybenzoate (DLT 26), 1,4-oxybut-2-enyl-bis(4-hydroxy-3-methoxybenzoate) (DLT27), 1,4-oxybut-2-ynyl bis(4-hydroxy-3-methoxybenzoate) (DLT 28), 6-(3-hydroxy-4-methoxy-benzoyl)oxyhexyl 3-hydroxy-4-methoxy-benzoate (DLT 29), [3-[(4-hydroxy-3-methoxy-benzoyl)oxymethyl]phenyl]methyl 4-hydroxy-3-methoxy-benzoate (DLT 25), 2-[bis[2-(4-hydroxy-3-methoxy-benzoyl)oxyethyl]amino]ethyl 4-hydroxy-3-methoxy-benzoate (DLT93), and [7-[(4-hydroxy-3-methoxy-benzoyl)oxymethyl]-2,6-dimethyl-3,5-dioxo-pyrazolo[1,2-a]pyrazol-1-yl]methyl 4-hydroxy-3-methoxy-benzoate (DLT 94).

In addition, the invention provides compounds having formula (V)

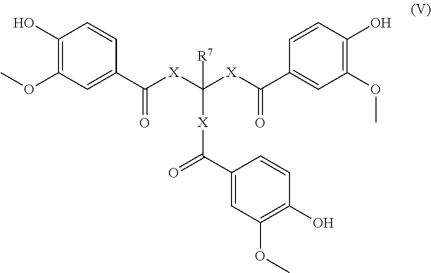

wherein X is selected from the group comprising O, O—C₁₋₆alkyl, NH, and N—C₁₋₆alkyl;
wherein R⁷ is selected from H, CO₂H, or C₁₋₆alkyl.

In addition, the invention provides compounds having general formula (VI).

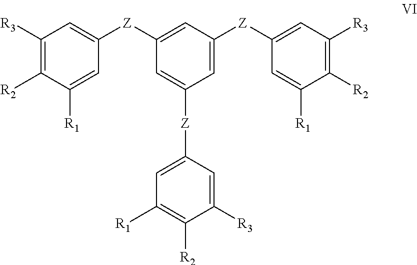

wherein Z is selected from the group comprising COCH₂CO, COCH₂CH₂, CH₂CH₂CO, CH₂COCH₂, COOCH₂, CONHCH₂, CON—C₁₋₆alkylCH₂, CONHCO, CON—C₁₋₆alkylCO, CH₂NHCH₂, CH₂N—C₁₋₆alkylCH₂, CH₂OCO, CH₂NHCO, CH₂N(C₁₋₆alkyl)CO, CH₂OCH₂, CH₂SCH₂, SO₂OCH₂, SO₂NHCH₂, SO₂N—C₁₋₆alkylCH₂;
and wherein R¹⁻³ can be each independently of each other ═H, OH, Halogen, C₁₋₈ alkoxyalkylene, OMe Ac, OAc, C₁₋₈ alkyl, NO₂;
and wherein two contiguous substituents among R¹⁻² can be together a dioxole.

In a more specific embodiment, the following compounds are envisaged by the invention: [3,5-bis-[(4-hydroxy-3-methoxy-benzoyl)-oxymethyl]-phenyl]-methyl-4-hydroxy-3-methoxy-benzoate (DLT95), [3,5-bis[(4-hydroxy-3-fluoro-benzoyl)oxymethyl]-phenyl]-methyl-4-hydroxy-3-fluoro-benzoate (DLT95-F), and [3,5-bis-[(4-hydroxy-3-chloro-benzoyl)-oxymethyl]-phenyl]methyl-4-hydroxy-3-chloro-benzoate (DLT95-Cl).

The invention further provides compounds having formula (IIa)

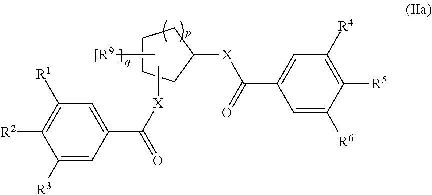

wherein X is selected from the group comprising O, O—C₁₋₆alkyl, NH, N—C₁₋₆alkyl;
wherein each R¹, R², R³, R⁴, R⁵, R⁶ is independently selected from the group comprising H, OH, C₁₋₈alkoxyC₁₋₆alkyl, C₁₋₆alkoxy, and halogen;
wherein p is an integer selected from 0, 1, 2, or 3; and
wherein R⁹ is selected from the group comprising OH, CO₂H, NH₂ and
wherein q is an integer selected from 0, 1, 2, or 3

In a preferred embodiment, X is oxygen, p is 2 and q is 0.

In a further preferred embodiment, X is NH, p is 2 and q is 0.

The invention further provides a pharmaceutical composition comprising one or more of the compound(s) of the invention and a pharmaceutically acceptable carrier.

The invention further provides compounds or a pharmaceutical composition according to the invention, for use as a medicament.

The invention further provides compounds or a pharmaceutical composition according to the invention for treating proliferative disorders (such as cancers), oxidative disorders, inflammatory disorders, Alzheimer's disease, Parkinson's disease, Pick's disease or for ameliorating symptoms of Down syndrome and sickle cell anemia disease.

The invention further provides the use of the compounds or the pharmaceutical composition according to the invention for the manufacturing of a medicament for treating proliferative disorders (such as cancers), oxidative disorders, inflammatory disorders, Alzheimer's disease, Parkinson's disease, Pick's disease or for ameliorating symptoms of Down syndrome and sickle cell anemia disease.

The invention further provides a method of treating proliferative disorders (such as cancers), oxidative disorders, inflammatory disorders, Alzheimer's disease, Parkinson's disease, Pick's disease or for ameliorating symptoms of Down syndrome and sickle cell disease anemia in a subject needing such therapy, comprising administering a therapeutically effective amount of one or more of the compound(s) or the pharmaceutical preparation according to the invention to a patient in need thereof. Optionally, the anti-cancer treatment is performed in combination with any of the cancer therapies selected from the group comprising of: chemotherapy, radiation therapy, immunotherapy, and/or gene therapy.

The invention further provides a method for treating oxidative and inflammatory disorders in a subject needing such therapy, comprising administering a therapeutically effective amount of one or more of the compound(s) or the pharmaceutical preparation according to the invention to said patient.

The invention further provides a method for treating oxidative and inflammatory disorders in a subject needing such therapy wherein the compound or the pharmaceutical preparation according to the invention is administered in combination with one or more active compounds, before, after or simultaneously with the administration of said compound or pharmaceutical composition.

In a preferred embodiment, the composition or the pharmaceutical preparation according to the invention is administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories, parenterally, for example subcutaneously, intramuscularly or intravenously in the form of solutions for injection or infusion, percutaneous or topical administration, for example in the form of ointments, tinctures, sprays or transdermal therapeutic systems, or the inhalative administration in the form of nasal sprays or aerosol mixtures, or, for example, microcapsules, implants or rods.

The invention also provides divanilloyl derivatives according to the invention for treating proliferative disorders such as neoplasma and cancers, dysplasia, premalignant or precancerous lesions, abnormal cell growths, benign tumours, malignant tumours, cancer or metastasis, wherein the cancer is selected from the group of: leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, and lymphoma.

In a preferred embodiment, the cancer is selected from the group of: leukemia, non-small cell lung cancer, small cell lung cancer, CNS cancer, melanoma, ovarian cancer, kidney cancer, prostate cancer, breast cancer, glioma, colon cancer, bladder cancer, sarcoma, pancreatic cancer, colorectal cancer, head and neck cancer, liver cancer, bone cancer, bone marrow cancer, stomach cancer, duodenum cancer, oesophageal cancer, thyroid cancer, hematological cancer, and lymphoma.

In a preferred embodiment, the patient is a mammal, e.g. a Horse, Rabbit, Mouse, Rat, Pig, Sheep, Cow or Dog. Preferably the subject is human.

In addition, the invention provides a pharmaceutical preparation comprising one or more of the compound(s) of the invention and a pharmaceutically acceptable carrier and/or additives selected from the group of: fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

In a further embodiment, the invention provides for a method of treating proliferative disorders in a subject needing such therapy, comprising administering a therapeutically effective amount of one or more of the compound(s) or the pharmaceutical preparation according to the invention to a patient in need thereof.

Alternatively, the invention provides for a method of treating oxidative and inflammatory disorders in a subject needing such therapy, comprising administering a therapeutically effective amount of one or more of the compound(s) or the pharmaceutical preparation according to the invention to said patient. In a specific embodiment, said treatment is performed in combination with any of the cancer therapies selected from the group comprising of: chemotherapy, radiation therapy, immunotherapy, and/or gene therapy. In a further embodiment, the compound or the pharmaceutical preparation according to the invention is administered in combination with one or more active compounds, before, after or simultaneously with the administration of the said compounds according to the invention. Administration can for example be orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories, parenterally, for example subcutaneously, intramuscularly or intravenously in the form of solutions for injection or infusion, percutaneous or topical administration, for example in the form of ointments, tinctures, sprays or transdermal therapeutic systems, or the inhalative administration in the form of nasal sprays or aerosol mixtures, or, for example, microcapsules, implants or rods.

In a further embodiment, the invention provides for a method for identifying agents for treating proliferative disorders, oxidative disorders, inflammatory disorders, Alzheimer's disease, Parkinson's disease, Pick's disease or for ameliorating symptoms of Down syndrome that inhibit the activity of one or more of the kinases selected from the group consisting of or comprising: Aurora A, B, or C, WEE1 and DYRK1A kinase; comprising the steps of

measuring the activity of said one or more kinases in the presence and absence of said agent, wherein a decrease in enzyme activity in the presence of the agent indicates that it is an inhibitory agent, and thus suitable for treating said disorders.

In a preferred embodiment of such a screening method, the agents are divanilloyl derivatives according to the invention. Preferably, said compounds are for treating proliferative disorders (such as cancers).

The present invention has permitted establishment of a rudimentary structure-activity relationship. Without wanting to be bound by any theory, it would appear that the anti-proliferative effect is depending on how the vanilloyl esters are linked to each other (e.g. carbon linear chain or cycloalkane diol structures). Even the stereoisomeric state is important, indicating that the positioning of the vanillic acid groups with respect to each other is important.

The best current hits, DLT12 and DLT4, display anti-proliferative, anti-migratory effects and anti-kinase effects which are ˜10 times more pronounced in cancer cells than in normal fibroblasts. Flow cytometry analyses has revealed that DLT4 does not induce pro-autophagic or pro-apoptotic effects in the cancer cell lines studied. Computer-assisted phase-contrast microscopy has however revealed that DLT compounds markedly impair both cell division and migration in the distinct cancer cell lines investigated but not in the normal fibroblasts. FIG. 1 illustrates the data obtained with respect to the human U373 glioblastoma model, while FIG. 2 illustrates the data obtained in human normal fibroblasts. When compared to other compounds known to impair ion channels in cancer cells, the data obtained with various DLT compounds, including for example DLT4, assessed using quantitative video-microscopy, are indeed suggestive of inhibition of ion channels. DLT-95 and its derivatives DLT-95-F and DLT-95-Cl were also shown to have a significant effect on the proliferation 7 different human cancer cell-lines.

### BRIEF DESCRIPTION OF THE DRAWINGS

FIG. 1: Illustrative phase contrast pictures obtained in an in vitro cellular imaging approach on human U373 glioblastoma cells untreated or treated with compounds according to the invention. Cellular imaging: pictures (time=72 h) of each cell line left untreated or treated with vanilloyl-ester compounds according to the invention (50 μM).

FIG. 2: Cellular imaging with human normal fibroblasts: pictures (time=72 h) of each cell line left untreated or treated with vanilloyl-ester compounds according to the invention (50 μM).

FIG. 3: DLT-11 kinase inhibition activity with 20 μM DLT11 was tested on 251 protein kinases. This figure illustrates only those kinases whose activity has been impaired by DLT 11 at a concentration (20 μM) below the IC₅₀ growth inhibitory values (obtained by means of the MTT colorimetric assay) relating to the in vitro DLT11 anti-tumor activity, which ranges between 22 and 71 μM depending on the cancer cell line analyzed (see Table 3). The activity of the Aurora kinases is the most impaired by DLT11 at 20 μM.

FIG. 4: In view of the results in FIG. 3, the inventors performed a broader analysis on impairment of Aurora kinase activity by different DLT compounds of the invention. This figure shows dose-response curves for Aurora A, B and C kinase activity inhibition with distinct DLT compounds and vanillic acid (AcVan) as a control, showing that unlike the control compound, all DLT compounds at a concentration of about 20 μM inhibit the activity of all three Aurora kinases with at least 50%.

FIG. 5: A similar study as in FIG. 4A was done for compound DLT-95 and its fluoride (DLT-95-F) and chloride (DLT-95-Cl) derivatives on Aurora A (A), B (B) and C (C), DYRK-1A (D) and WEE1 kinases (E) Vanillic acid (AcVan) was used as the control substance. Unlike the control compound, DLT-95-F and DLT-95-CL at a concentration of 57 μM inhibit activity of all five kinases with at least 50%, while DLT-95 itself has an inhibitory activity which is more specific for the 3 Aurora kinases.

FIG. 6: A similar study as in FIG. 1 was done for compound DLT-95 and its fluoride (DLT-95-F) and chloride (DLT-95-Cl) substituents. The compounds were administered in their respective IC₅₀ concentrations given is the figures. As becomes clear from the figure, after 72 hours, the DLT-95 compound is the most effective in reducing U373 cell growth, while DLT-95-CL and -F are less effective but nonetheless still result in a significant reduction of the growth of the U373 cells as compared to the control, e.g. U373 cells that have been left untreated.

### DETAILED DESCRIPTION OF THE PREFERRED EMBODIMENTS

The invention provides new compounds with an anti-cancer activity. Said compounds are defined as being di- and tri-vanilloyl derivatives of the general formula (I):

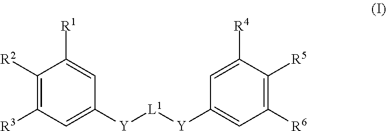

wherein R¹⁻⁶ can be each independently of each other ═H, OH, C₁₋₈ alkoxyalkylene, OMe, Ac, OAc, C₁₋₈ alkyl, NO₂ or a halogen such as F or Cl, and wherein two contiguous substituents among R¹⁻³ can be together a dioxole;
wherein Y is selected from the group comprising COO, tetrazole, OCO, OCOO, CONR¹⁰, NR¹⁰CO, OCONR¹⁰, NR¹⁰COO, NR¹⁰CONR¹⁰, COCH₂CO, COCH₂CH₂, CH₂CH₂CO, CH₂COCH₂, COOCH₂, CONHCH₂, CON—C₁₋₆alkylCH₂, CONHCO, CON—C₁₋₆alkylCO, CH₂NHCH₂, CH₂N—C₁₋₆alkylCH₂, CH₂OCO, CH₂NHCO, CH₂N(C₁₋₆alkyl)CO, CH₂OCH₂, CH₂SCH₂, SO₂OCH₂, SO₂NHCH₂, and SO₂N—C₁₋₆alkylCH₂,
wherein R¹⁰=H, C₁₋₄ Alkyl,
wherein L¹=C₁₋₈alkylene, preferably C₅₋₁₀ alkylene; or
wherein L¹=(CH₂)ₙ, wherein n is an integer selected from 2-10; or
wherein

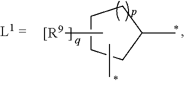

or
wherein

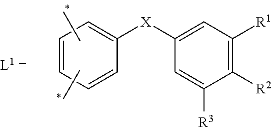

(* The asterisk is used herein to indicate the point at which a mono- or bivalent radical depicted is connected to the structure to which it relates and of which the radical forms part),
wherein R⁹ is selected from the group comprising OH, CO₂H, and NH₂ and
wherein q is an integer selected from 0, 1, 2, or 3;
each group being optionally substituted with one, two or three substituents each independently selected form the group comprising C₁₋₆alkyl, CO₂H, vanillic acid, amine, and C₁₋₆alkyloxycarbonyl, wherein p is an integer selected from 0, 1, 2, or 3; or
stereoisomeric forms thereof and the pharmaceutically acceptable addition salts, hydrates or solvates thereof, for treating proliferative disorders, oxidative disorders, inflammatory disorders, Alzheimer's disease, Parkinson's disease, Pick's disease or for ameliorating symptoms of Down syndrome.

More particularly, the divanilloyl derivatives are esters or amides of the general formula (II):

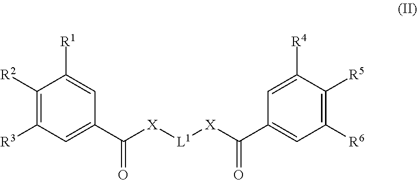

wherein X is selected from the group comprising O, O—C₁₋₆alkyl, NH, and N—C₁₋₆alkyl;
wherein each R¹, R², R³, R⁴, R⁵, R⁶ is independently selected from the group comprising H, OH, C₁₋₈alkoxyC₁₋₆alkyl, C₁₋₆alkoxy, and halogen, such as e.g. F and Cl;
wherein L¹ is a group selected from C₁₋₈alkylene,

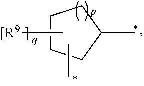

or

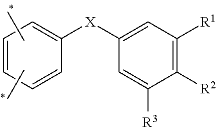

(* The asterisk is used herein to indicate the point at which a mono- or bivalent radical depicted is connected to the structure to which it relates and of which the radical forms part);
each group being optionally substituted with one, two or three substituents each independently selected form the group comprising C₁₋₆alkyl, CO₂H, vanillic acid, amine, and C₁₋₆alkyloxycarbonyl,
wherein p is an integer selected from 0, 1, 2, or 3;
wherein R⁹ is selected from the group comprising OH, CO₂H, and NH₂ and
wherein q is an integer selected from 0, 1, 2, or 3;
or stereoisomeric forms thereof, and the pharmaceutically acceptable addition salts, hydrates or solvates thereof; and their use for treating proliferative disorders, oxidative disorders, inflammatory disorders, Alzheimer's disease, Parkinson's disease, Pick's disease or for ameliorating symptoms of Down syndrome and sickle cell anemia disease.

Said compounds of the invention can be used for treating proliferative disorders (such as cancers), oxidative disorders, inflammatory disorders, Alzheimer's disease, Parkinson's disease, Pick's disease or for ameliorating symptoms of Down syndrome and sickle cell anemia disease.

Preferred embodiments are:

### I—Divanilloyl Esters or Amides on a Linear Carbon Chain (Formula (III))

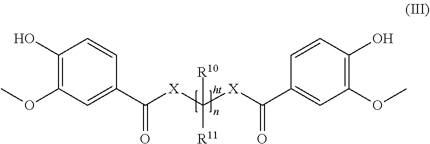

wherein X is selected from the group comprising O, O—C₁₋₆alkyl, NH, and N—C₁₋₆alkyl;
wherein n is an integer selected from 1, 2, 3, 4, 5, 6, 7, or 8.
wherein R¹⁰ and R¹¹ are, each independently selected from the group comprising H, CO₂H, or C₁₋₆alkyl and vanillic acid.

Specific examples of compounds of the invention are (cf. also Table 1):

DLT1: wherein X=O, R¹⁰ and R¹¹=H and n=2
DLT2: wherein X=O, R¹⁰ and R¹¹=H and n=3
DLT10: wherein X=O, R¹⁰ and R¹¹=H and n=4
DLT3: wherein X=O, R¹⁰ and R¹¹=H and n=5
DLT11: wherein X=O, R¹⁰ and R¹¹=H and n=6
DLT12: wherein X=O, R¹⁰ and R¹¹=H and n=7
DLT13: wherein X=O, R¹⁰ and R¹¹=H and n=8

### II—Divanilloyl Esters or Amides on a Cyclic Carbon Chain (Formula (IV))

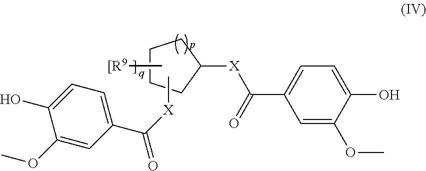

wherein X is selected from the group comprising O, O—C₁₋₆alkyl, NH, and N—C₁₋₆alkyl;
wherein p is an integer selected from 0, 1, 2, or 3; and
R⁹ is selected from the group comprising OH, CO₂H, NH₂ and q is an integer selected from 0, 1, 2, or 3.

Specific examples of compounds of the invention of the cyclohexane esters type (X=O, p=2 and q=0) are (cf. also Table 1):

DLT4: which is 1, 2 racemic trans
DLT7: which is 1, 2 trans S,S
DLT8 which is 1,2 trans R,R
DLT9: which is 1,2 cis
DLT5: which is 1,3 cis-trans 3:7

### III—Trivanilloyl Esters or Amides (Formula (V))

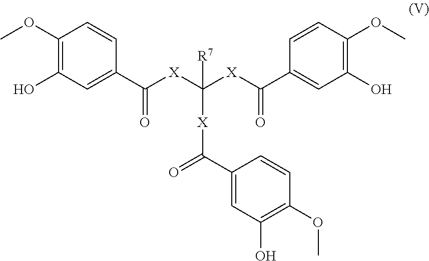

wherein X is selected from the group comprising O, O—C₁₋₆alkyl, NH, N—C₁₋₆alkyl;
wherein R⁷ is selected from H, CO₂H, or C₁₋₆alkyl.

Further specific examples of compounds according to the invention are:

### [2-[(4-hydroxy-3-methoxy-benzoyl)oxymethyl]phenyl]methyl 4-hydroxy-3-methoxy-benzoate; methanol) (DLT 24)

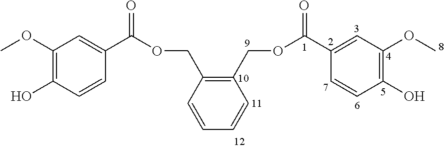

### 6-(3,4-dimethoxybenzoyl)oxyhexyl 3,4-dimethoxybenzoate (DLT 26)

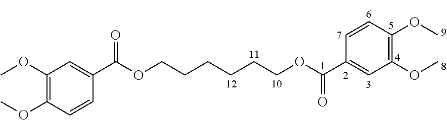

### 1,4-oxybut-2-enyl-bis(4-hydroxy-3-methoxybenzoate) (or [(E)-4-(4-hydroxy-3-methoxy-benzoyl)oxybut-2-enyl]4-hydroxy-3-methoxy-benzoate; methanol) (DLT27). Mixture of Cis and Trans

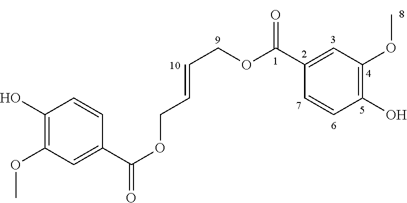

### 1,4-oxybut-2-ynyl bis(4-hydroxy-3-methoxybenzoate) (DLT 28)

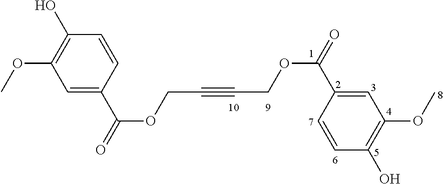

### 6-(3-hydroxy-4-methoxy-benzoyl)oxyhexyl 3-hydroxy-4-methoxy-benzoate (DLT 29)

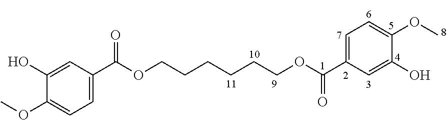

### [3-[(4-hydroxy-3-methoxy-benzoyl)oxymethyl]phenyl]methyl 4-hydroxy-3-methoxy-benzoate (DLT 25)

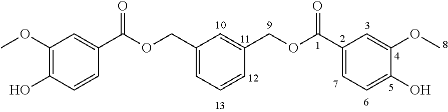

### 2-[bis[2-(4-hydroxy-3-methoxy-benzoyl)oxyethyl]amino]ethyl 4-hydroxy-3-methoxy-benzoate (DLT93)

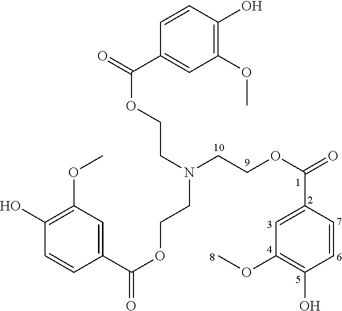

A further specific example of compound according to the invention is the bimane derivative below. Such a compound is prone to have fluorescent properties, suitable for biological probing purpose.

### [7-[(4-hydroxy-3-methoxy-benzoyl)oxymethyl]-2,6-dimethyl-3,5-dioxo-pyrazolo[1,2-a]pyrazol-1-yl]methyl 4-hydroxy-3-methoxy-benzoate (DLT 94)

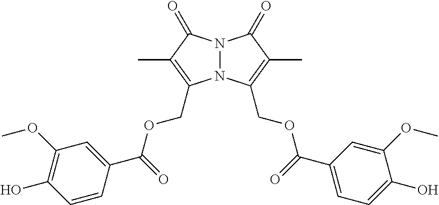

This compound could be synthesized according to exactly the same alkylation strategy from the commercially available dibromobimane and vanillic acid.

### IV Trivanilloyl Derivatives of General Formula (VI)

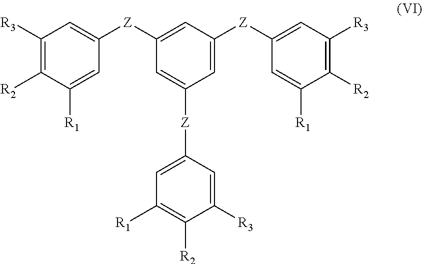

wherein Z is selected from the group comprising COCH₂CO, COCH₂CH₂, CH₂CH₂CO, CH₂COCH₂, COOCH₂, CONHCH₂, CON—C₁₋₆alkylCH₂, CONHCO, CON—C₁₋₆alkylCO, CH₂NHCH₂, CH₂N—C₁₋₆alkylCH₂, CH₂OCO, CH₂NHCO, CH₂N(C₁₋₆alkyl)CO, CH₂OCH₂, CH₂SCH₂, SO₂OCH₂, SO₂NHCH₂, SO₂N—C₁₋₆alkylCH₂;
and wherein R¹⁻³ can be each independently of each other ═H, OH, Halogen, O₁₋₈ alkoxyalkylene, OMe Ac, OAc, C₁₋₈ alkyl, NO₂;
and wherein two contiguous substituents among R¹⁻³ can be together a dioxole.

Three compounds have already been synthesized by a single step in this series:

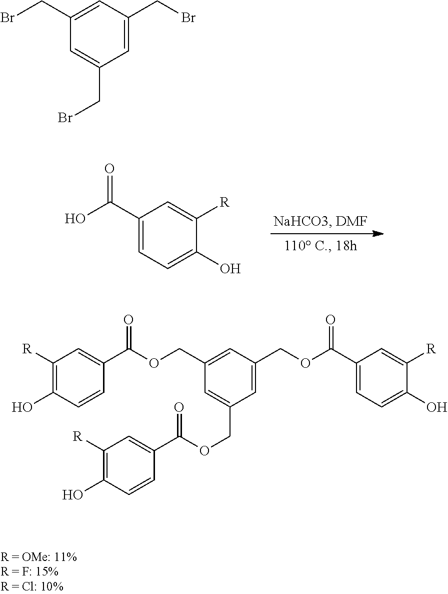

### [3,5-bis[(4-hydroxy-3-methoxy-benzoyl)oxymethyl]phenyl]methyl 4-hydroxy-3-methoxy-benzoate. DLT95

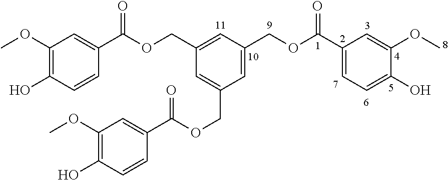

Yield=11%, R_{f}=0.23 (Cyclohexane/AcOEt: 5/5), then precipitation from CH₂Cl₂. RP-HPLC: purity=100% (254 nm), t_{R}=4.04 min; ¹H NMR (DMSO-d6): δ 9.99 (bs, 3H, OH), 7.50 to 7.45 (m, 9H, H-3, H-7, H-11), 6.85 (d, ³J_{6,7}=8.1, 3H, H-6), 5.34 (s, 6H, H-9), 3.78 (s, 9H, H-8). ¹³C NMR (DMSO-d6): δ 165.3 (C-1), 151.6 (C-5), 147.3 (C-4), 137.1 (C-10), 126.5 (C-11), 123.5 (C-7), 120.1 (C-2), 115.1 (C-6), 112.4 (C-3), 65.2 (C-9), 55.5 (C-8). Mp: 161° C. Anal. Calcd for C₃₃H₃₀O₁₂.3/2CH₂Cl₂: C, 59.89; H, 4.68. Found: C, 59.84; H, 4.72 (equivalent to 8% of CH₂Cl₂ w/w). MS (ESI+) m/z 641.1625 (MNa+), 0.6 ppm. IR-FT: 3378.86; 2941.00; 1701.97; 1597.35; 1528.19; 1516.96.

### [3,5-bis[(4-hydroxy-3-fluoro-benzoyl)oxymethyl]phenyl]methyl 4-hydroxy-3-fluoro-benzoate. DLT95-F

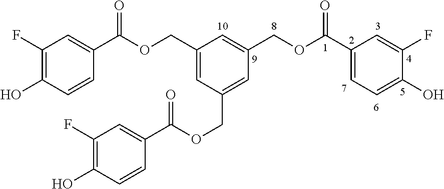

Yield=15%, R_{f}=0.4 (CH₂Cl₂/MeOH: 9/1), RP-HPLC: purity=98.4% (254 nm), t_{R}=3.96 min, ¹H NMR (DMSO-d6): δ 10.91 (bs, 3H, OH), 7.68 to 7.7.65 (m, 6H, H-3, H-7), 7.51 (s, 3H, H-10), 7.03 (t, ³J_{6,7}=⁴J_{6-F}=9.0, 2H, H-6), 5.34 (s, 6H, H-8). ¹³C NMR (DMSO-d6): δ 164.5 (d, ⁴J_{1-F}=2.3, C-1), 150.4 (d, ¹J_{4-F}=240.7, C-4), 149.1 (d, ²J_{5-F}=12.0, C-5), 137.0 (C-9), 126.8 (C-10), 126.7 (d, ⁴J_{7-F}=2.3, C-7), 120.5 (d, ³J_{2-F}=6.0, C-2), 117.6 (d, ³J_{6-F}=3.0, C-6), 117.0 (d, ²J_{3-F}=19.5.0, C-3), 65.6 (C-8). Mp: 88° C. Anal. Calcd for C₃₀H₂₁F₃O₉1/4CH₂Cl₂: C, 60.18; H, 3.59. Found: C, 60.16; H, 4.00. IR-FT: 3382.35; 2957.89; 1702.31; 1617.81; 1597.99; 1518.77.

### [3,5-bis[(4-hydroxy-3-chloro-benzoyl)oxymethyl]phenyl]methyl 4-hydroxy-3-chloro-benzoate. DLT95-Cl

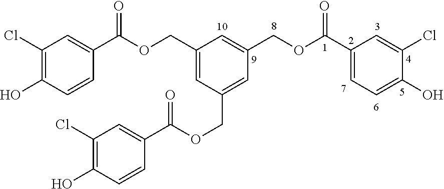

Yield=10%, R_{f}=0.27 (CH₂Cl₂/MeOH: 98/2), RP-HPLC: purity=89.8% (210 nm), t_{R}=2.73 min, ¹H NMR (DMSO-d6): δ 11.3 (bs, 3H, OH), 7.88 (d, ⁴J_{3,7}=1.8, 3H, H-3), 7.79 (dd, ³J_{6,7}=8.7, ⁴J_{3,7}=1.8, 3H, H-7), 7.51 (s, 3H, H-10), 7.05 (d, ³J_{6,7}=8.7, 3H, H-6), 5.34 (s, 6H, H-8). ¹³C NMR (DMSO-d6): δ 164.3 (C-1), 157.7 (C-5), 136.9 (C-4), 131.0 (C-3), 129.8 (C-7), 126.9 (C-2), 121.1 (C-4), 119.9 (C-10), 116.4 (C-6). Mp: 178° C. Anal. Calcd for C₃₀H₂₁Cl₃O₉1/8CH₂Cl₂: C, 56.32; H, 3.33. Found: C, 56.43; H, 3.58. IR-FT: 3393.09; 2961.56; 1690.44; 1601.50; 1579.04; 1500.77.

Other compounds of the general formula VI family can be synthesized using similar synthesis processes.

The inventors have established that simplified Burkinabin-like chemical compounds have certain anti-cancer treatment properties. These properties appear to be depending on 1) the distance between the two vanillic acid components, i.e. the length of the linear carbon chains in between both vanillic acid groups appears to be important; 2) the relative position of the vanillic acid groups, i.e. in the same plane or not, depending on the stereoisomery of the structures; 3) on the number of vanillic acids present in the structure, i.e. 2 in the divanilloyl esters or amides or 3 in the trivanilloyl esters or amides. A combination of all three factors influences the activity of the compounds. The inventors therefore investigated the anti-cancer activity of several newly synthesized vanilloyl esters. Additional examples of such compounds can be found in table 1 below.

Accordingly, the present invention provides a method for the treatment of cancer comprising administering to an individual an effective amount of at least one compound or pharmaceutical composition of the invention as an active ingredient, such that the cancer is treated. By way of example, in an embodiment of the invention, cancer is treated in a subject in need of treatment by administering to the subject a therapeutically effective amount of at least one compound of the invention, effective to treat the cancer.

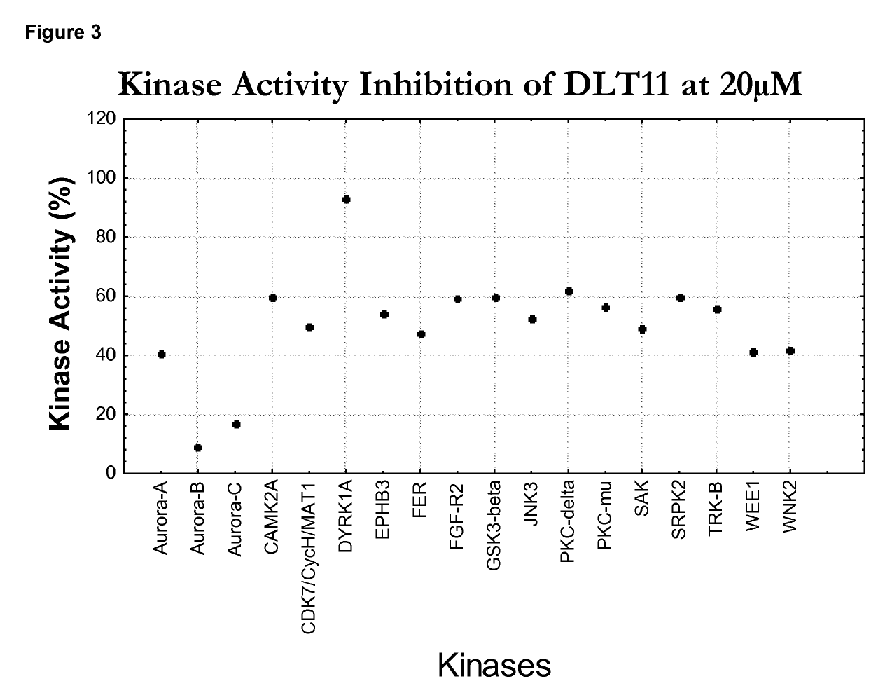

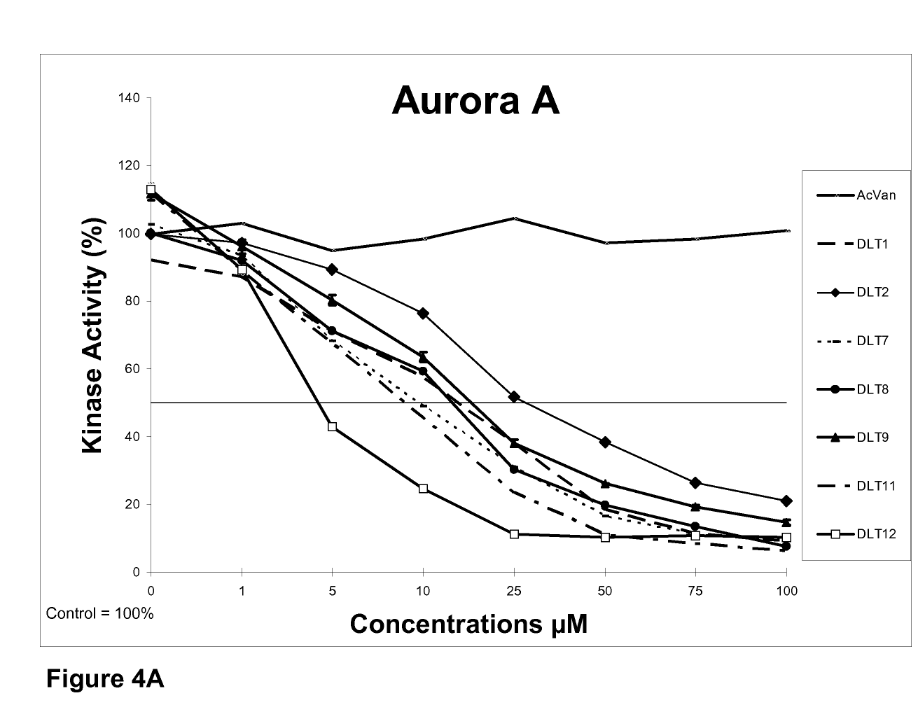

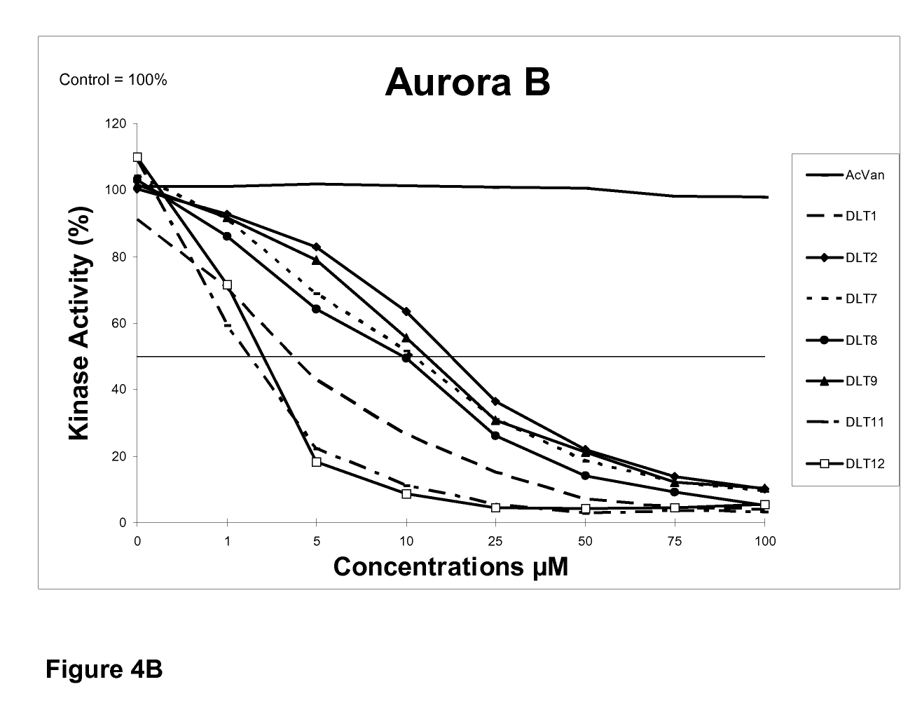

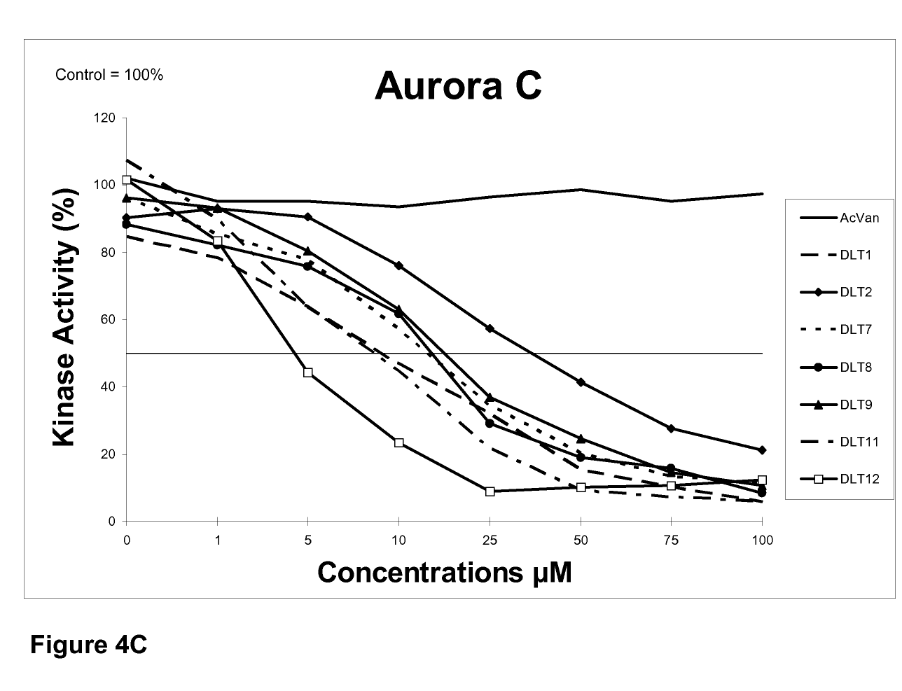

In addition, the inventors have embarked on a route to identify the actual targets of the compounds of the invention and have established that certain kinases, known to be involved in proliferation disorders are inhibited by some of the compounds of the invention. The results are presented in examples 3 to 5 and in FIGS. 3 and 4.

From these results, it becomes clear that the DLT11 compound inhibits 2 Aurora kinases for more than 80%, but also inhibits 15 further kinases for 40 to 60% 1 kinase is inhibited by for 5%. The Dyrk1A (dual specificity tyrosine-phosphorylated and regulated kinase 1a) is inhibited by DLT11 for only 5%, but also by two other tested compounds of the invention, namely DLT1 (51% inhibition) and DLT5 (26% inhibition) (Table 6). Of >250 kinases analyzed, the inventors showed that the activity of the Aurora A, B and C kinases has been impaired by DLT compounds most markedly (Table 6 and FIG. 4).

This enabled the inventors to construct a screening assay for further compounds having a similar effect, without the need of cellular or in vivo experimentation. The idea is to measure the activity of a certain kinase, shown to be inhibited by one or more of the compounds of the invention in the presence and absence of a candidate agent, wherein an inhibition of said kinase is indicative of the anti-proliferative effect of the agent. This way, high throughput screening assays or platforms can be established to identify lead compounds, which can then be further tested in vitro and in vivo.

The invention thus additionally provides a method for screening or a method to identify agents or compounds that have a use in treatment of proliferative disorders (such as cancers), but also for oxidative disorders, inflammatory disorders, Alzheimer's disease, Parkinson's disease, Pick's disease or for ameliorating symptoms of Down syndrome and sickle cell anemia disease comprising the steps of:

a) providing a kinase and measuring its activity
b) contacting said kinase with a candidate agent and re-measuring the activity of said kinase, and
c) comparing the activity of said kinase between steps a) and b), wherein a decrease of the activity of said kinase in step b) compared to step a) indicates that the candidate agent has an anti-proliferative effect.

In a preferred embodiment, said kinase is Dyrk1A (dual specificity tyrosine-phosphorylated and regulated kinase 1a) or CK-1 (casein kinase 1) or Aurora, most preferably Aurora A, B and C.

The candidate agents can be any molecule or compound binding to and acting on said kinase, e.g. antibodies, aptamers, specifically interacting small molecules or chemical compounds, specifically interacting proteins, and other molecules that specifically bind to one of the biomarkers.

The inhibitory effect can is preferably 10% or more, 15% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, 99% or more or can be total inhibition.

In a preferred embodiment, said percentage of inhibition is at least 40% or at least 50%.

In a preferred embodiment of the screening method, the compounds to be screened are Divanilloyl derivatives disclosed in the present invention to have anti-proliferative effects for the first time.

Overexpression of the DYRK1A kinase has been implicated in the multiple diseases or disorders or syndromes: cancer, tumorigenesis and uncontrolled proliferation (Laguna A et al., Dev Cell. 2008 December; 15(6):841-53); Alzheimer disease, Down syndrome, Pick disease (Ferrer I et al., Neurobiol Dis. 2005 November; 20(2):392-400; Kimura R et al., Hum Mol Genet. 2007 Jan. 1; 16(1):15-23) and Down Syndrome (Guedj F et al., PLoS ONE. 2009; 4(2):e4606; Lepagnol-Bestel A M et al., Hum Mol Genet. 2009 Feb. 12; Wegiel J et al., Acta Neuropathol. 2008 October; 116(4):391-407). The compounds according to the invention can thus also be used in the treatment or for the amelioration of the effects of diseases correlated with uncontrolled or increased DYRK1A expression such as cancer, proliferation disorders, Alzheimer disease, Down syndrome, Pick disease and Down's syndrome.

Overexpression of the Aurora A, B an C kinases has been implicated in multiple diseases or disorders or syndromes such as cancer, tumorigenesis and uncontrolled proliferation (Carjaval R D et al., Clin Cancer Res 2006; Fu J et al., Mol Cancer Res 2007; Vader G & Lens S M A, Biochim Biophys Acta 2008).

The term “anti-migratory” as used herein refers to the ability of a compound or pharmaceutical composition of the invention to stop the migration of cells, required to go away from the neoplastic tumor tissue, and thus to reduce the colonization of new tissues by these cells.

The term “treating” as used herein includes treating any one or more of the conditions underlying or characteristic of cancer. Treatment of cancer means administration of a medicament in the form of a compound or pharmaceutical composition of the invention with the result that cancer is stabilized, reduced or the patient is cured.

As used herein, the singular forms “a”, “an”, and “the” include both singular and plural referents unless the context clearly dictates otherwise. By way of example, “an antibody” refers to one or more than one antibody; “an antigen” refers to one or more than one antigen.

The terms “comprising”, “comprises” and “comprised of” as used herein are synonymous with “including”, “includes” or “containing”, “contains”, and are inclusive or open-ended and do not exclude additional, non-recited members, elements or method steps.

The term “about” as used herein when referring to a measurable value such as a parameter, an amount, a temporal duration, and the like, is meant to encompass variations of +/−20% or less, preferably +/−10% or less, more preferably +/−5% or less, even more preferably +/−1% or less, and still more preferably +/−0.1% or less from the specified value, insofar such variations are appropriate to perform in the disclosed invention.

All documents cited in the present specification are hereby incorporated by reference in their entirety. In particular, the teachings of all documents herein specifically referred to are incorporated by reference.

The present invention concerns methods and compounds or pharmaceutical compositions useful for the treatment of proliferative disorders.

By “proliferative disease or disorder” is meant all neoplastic cell growth and proliferation, whether malignant or benign, including all transformed cells and tissues and all cancerous cells and tissues. Proliferative diseases or disorders include, but are not limited to, premalignant or precancerous lesions, abnormal cell growths, benign tumours, malignant tumours, and cancer.

Additional examples of proliferative diseases and/or disorders include, but are not limited to neoplasms, whether benign or malignant, located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital tract. In a preferred embodiment, the proliferative disorder involves tumour.

As used herein, the terms “tumour” or “tumour tissue” refer to an abnormal mass of tissue that results from excessive cell division. A tumour or tumour tissue comprises “tumour cells” which are neoplastic cells with abnormal growth properties and no useful bodily function. Tumours, tumour tissue and tumour cells may be benign or malignant. A tumour or tumour tissue may also comprise “tumour-associated non-tumour cells”, e.g., vascular cells which form blood vessels to supply the tumour or tumour tissue. Non-tumour cells may be induced to replicate and develop by tumour cells, for example, the induction of angiogenesis in a tumour or tumour tissue. In another preferred embodiment, the proliferative disorder involves malignancy or cancer.

As used herein, the term “malignancy” refers to a non-benign tumour or a cancer. As used herein, the term “cancer” connotes a type of proliferative disease which includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include: squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as CNS cancer, melanoma, head and neck cancer, bone cancer, bone marrow cancer, duodenum cancer, oesophageal cancer, thyroid cancer, hematological cancer. The term “cancer” includes primary malignant cells or tumours (e.g., those whose cells have not migrated to sites in the subject's body other than the site of the original malignancy or tumour) and secondary malignant cells or tumours (e.g., those arising from metastasis, the migration of malignant cells or tumour cells to secondary sites that are different from the site of the original tumour).

Preferably, said cancer is selected from non-small cell lung cancer, CNS cancer, melanoma, ovarian cancer, kidney cancer, prostate cancer, breast cancer, colon cancer, bladder cancer, sarcoma, pancreatic cancer, colorectal cancer, head and neck cancer, liver cancer, stomach cancer, oesophageal cancer, or lymphoma.

Most preferably, said cancer is selected from colon cancer; prostate cancer; breast cancer; head and neck cancer; glioma, preferably glioblastoma or non-small-cell lung cancer (NSCLC) and apoptosis resistant cancer cells in the general meaning of the term.

Apoptosis resistant cancer cells means cancer cells that are resistant to apoptosis and that cannot be killed by pro-apoptotic drugs.

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumours, Breast Cancer, Cancer of the Renal Pelvis and Urethra, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Glioblastoma, Childhood Cerebellar Astrocytoma, Childhood Cerebral Astrocytoma, Childhood Extracranial Germ Cell Tumours, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumours, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumours, Exocrine Pancreatic Cancer, Extracranial Germ Cell Tumour, Extragonadal Germ Cell Tumour, Extrahepatic Bile Duct Cancer, Eye Cancer, Female Breast Cancer, Gaucher's Disease, Gallbladder Cancer, Gastric Cancer, Gastrointestinal Carcinoid Tumour, Gastrointestinal Tumours, Germ Cell Tumours, Gestational Trophoblastic Tumour, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumour, Ovarian Low Malignant Potential Tumour, Pancreatic Cancer, Paraproteinemias, Purpura, Parathyroid Cancer, Penile Cancer, Pheochromocytoma, Pituitary Tumour, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Urethra Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumours, T-Cell Lymphoma, Testicular Cancer, Thymoma, Thyroid Cancer, Transitional Cell Cancer of the Renal Pelvis and Urethra, Transitional Renal Pelvis and Urethra Cancer, Trophoblastic Tumours, Urethra and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumour, and any other proliferative disease, besides neoplasia, located in an organ system listed above.

In a further embodiment, the proliferative disorder is premalignant condition. Premalignant conditions are known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell 1976 (Basic Pathology, 2d Ed., W. B. Saunders Co., Philadelphia, pp. 68-79).

“Hyperplasia” is a form of controlled cell proliferation, involving an increase in cell number in a tissue or organ, without significant alteration in structure or function. Hyperplastic disorders which can be treated by the method of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

“Metaplasia” is a form of controlled cell growth in which one type of adult or fully differentiated cell substitutes for another type of adult cell. Metaplastic disorders which can be treated by the method of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

“Dysplasia” is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated by the method of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders include, but are not limited to, benign dysproliferative disorders (e.g., benign tumours, fibrocystic conditions, tissue hypertrophy, intestinal polyps, colon polyps, and oesophageal dysplasia), leukoplakia, keratoses, Bowen's disease, Farmer's Skin, solar cheilitis, and solar keratosis.

In preferred embodiments, the proliferative disorder is chosen from glioma, preferably glioblastoma; prostate cancer; non-small-cell lung cancer (NSCLC); melanoma, head and neck cancer, pancreas cancer or colon cancer. By showing the anti-proliferative effect of the compounds of the invention on cell-lines derived from each of these cancer-types, the inventors realised that the above cancer types can particularly benefit from the methods and agents of the invention.

As used herein, the term “glioma” refers to its art-recognised connotation. By virtue of further illustration and not limitation, the term “glioma” refers to a tumour originating in the neuroglia of the brain or spinal cord. Gliomas can be derived from glial cell types, such as, e.g., astrocytes and oligodendrocytes, thus gliomas include astrocytomas and oligodendrogliomas, as well as anaplastic gliomas, glioblastomas, and ependymonas. Astrocytomas and ependymomas can occur in all areas of the brain and spinal cord in both children and adults. Oligodendrogliomas typically occur in the cerebral hemispheres of adults. Malignant astrocytic gliomas are associated with the worst prognoses because of their ability to infiltrate diffusely into the normal brain parenchyma and include World Health Organization (WHO) grades II, Ill and grade IV tumors.

As used herein, the term “glioblastoma” refers to its art-recognised connotation. By virtue of further illustration and not limitation, glioblastoma may also be known as “glioblastoma multiforme” (GBM) or as “grade 4 astrocytoma” and represents perhaps the most common and aggressive type of malignant primary brain tumour.

As used herein, the term “prostate cancer” (CaP) refers to its art-recognised connotation. By virtue of illustration and not limitation, the term “prostate cancer” refers to both the appearance of a palpable tumour of the prostate, and also to microscopically detectable neoplastic or transformed cells in the prostate gland. In the latter case, the said cytologically-detectable prostate cancer may be asymptomatic, in that neither the patient nor the medical practitioner detects the presence of the cancer cells. Cancer cells are generally found in the prostates of men who live into their seventies or eighties, however not all of these men develop prostate cancer. In the event that prostate cancer metastasises to additional sites distal to the prostate, the condition is described as metastatic cancer (MC), to distinguish this condition from organ-confined prostate cancer. CaP fatality typically results from metastatic dissemination of prostatic adenocarcinoma cells to distant sites, usually in the axial skeleton.

The term “non-small-cell lung cancer” (NSCLC) refers to its art-recognised connotation. By means of exemplification and not limitation, the term encompasses any of subtypes thereof, i.e., adenocarcinoma of the lung, squamous cell carcinoma of the lung and large cell carcinoma of the lung.

The term “colon cancer” refers to its art-recognised connotation. By means of illustration and not limitation, the term “colon cancer” refers to cancers arising in the large intestine (including both the colon and rectum) of any histologic type, including but not limited to malignant epithelial tumours. As used herein the term colon cancer thus encompasses colorectal cancer. Malignant epithelial tumours of the large intestine may be divided into five major histologic types: adenocarcinoma, mucinous adenocarcinoma (also termed colloid adenocarcinoma), signet ring adenocarcinoma, scirrhous tumours and carcinoma simplex. Colon cancer is staged using any of several classification systems known in the art. The Dukes system is one of the most often employed staging systems. See Dukes and Bussey 1958 (Br J Cancer 12: 309).

The present invention also provides methods of treating proliferative disorders in a subject needing such therapy, comprising administering a therapeutically effective amount of the compound or the pharmaceutical composition of the invention.

The present invention also provides methods of treating oxidative and inflammatory disorders in a subject needing such therapy, comprising administering a therapeutically effective amount of the compound or the pharmaceutical composition of the invention.

Except when noted, “subject” or “patient” are used interchangeably and refer to animals, preferably vertebrates, more preferably mammals, and specifically includes human patients and non-human mammals. “Mammalian” subjects include, but are not limited to, humans, domestic animals, commercial animals, farm animals, zoo animals, sport animals, pet and experimental animals such as dogs, cats, guinea pigs, rabbits, rats, mice, horses, cattle, cows; primates such as apes, monkeys, orang-utans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Accordingly, “subject” or “patient” as used herein means any mammalian patient or subject to which the compositions of the invention can be administered. Preferred patients are human subjects.

As used herein, the terms “treat” or “treatment” refer to both therapeutic treatment and prophylactic or preventative measures, wherein the object is to prevent or slow down (lessen) an undesired physiological change or disorder, such as the development or spread of proliferative disease, e.g., cancer. Beneficial or desired clinical results include, but are not limited to, alleviation of symptoms, diminishment of extent of disease, stabilised (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, a phrase such as “a subject in need of treatment” includes subjects, such as mammalian subjects, that would benefit from treatment of a given condition, preferably a proliferative disease, such as, e.g., cancer, e.g., as above.

Such subjects will typically include, without limitation, those that have been diagnosed with the condition, preferably a proliferative disease, e.g., cancer, those prone to have or develop the said condition and/or those in whom the condition is to be prevented.

The term “therapeutically effective amount” refers to an amount of a compound or pharmaceutical composition of the invention effective to treat a disease or disorder in a subject, i.e., to obtain a desired local or systemic effect and performance. By means of example and not limitation, in the case of proliferative disease, e.g., cancer, therapeutically effective amount of a drug may reduce the number of cancer cells; reduce the tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; enhance efficacy of another cancer therapy; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR). The term thus refers to the quantity of compound or pharmaceutical composition that elicits the biological or medicinal response in a tissue, system, animal, or human that is being sought by a researcher, veterinarian, medical doctor or other clinician, which includes alleviation of the symptoms of the cancer being treated. In particular, these terms refer to the quantity of compound or pharmaceutical composition according to the invention which is necessary to prevent, cure, ameliorate, or at least minimize the clinical impairment, symptoms, or complications associated with cancer in either a single or multiple doses.

The compound or the pharmaceutical composition of the invention may be used alone or in combination with any of the cancer therapies selected from the group comprising chemotherapy, radiation therapy, immunotherapy, and/or gene therapy. As used herein the term “cancer therapy” is meant to encompass radiation therapy, chemotherapy, immunotherapy, gene-based therapy, surgery, as well as combinations thereof.

In another preferred embodiment the compound or the pharmaceutical composition of the invention may be used alone or in combination with one or more active compounds that are suitable in the treatment of cancer, preferably glioma, preferably glioblastoma; prostate cancer; NSCLC; or colon cancer. The term “active compound” refers to a compound other than the agents of the invention which is used to treat cancer. The active compounds may preferably be selected from the group comprising radiation therapeutics, chemotherapeutics including but not limited to temozolomide, vincristine, vinorelbine, procarbazine, carmustine, lomustine, taxol, taxotere, tamoxifen, retinoic acid, 5-fluorouracil, cyclophosphamide and thalidomide.

The compound or the pharmaceutical composition of the invention can thus be administered alone or in combination with one or more active compounds. The latter can be administered before, after or simultaneously with the administration of the said agent(s).

A further object of the invention are pharmaceutical preparations which comprise a therapeutically effective amount of the compound of the invention as defined herein, or pharmaceutically acceptable salts thereof, and a pharmaceutically acceptable carrier, i.e., one or more pharmaceutically acceptable carrier substances and/or additives, e.g., buffers, carriers, excipients, stabilisers, etc.

The term “pharmaceutically acceptable” as used herein is consistent with the art and means compatible with the other ingredients of a pharmaceutical composition and not deleterious to the recipient thereof.

The term “pharmaceutically acceptable salts” as used herein means an inorganic acid addition salt such as hydrochloride, sulfate, and phosphate, or an organic acid addition salt such as acetate, maleate, fumarate, tartrate, and citrate. Examples of pharmaceutically acceptable metal salts are alkali metal salts such as sodium salt and potassium salt, alkaline earth metal salts such as magnesium salt and calcium salt, aluminum salt, and zinc salt. Examples of pharmaceutically acceptable ammonium salts are ammonium salt and tetramethylammonium salt. Examples of pharmaceutically acceptable organic amine addition salts are salts with morpholine and piperidine. Examples of pharmaceutically acceptable amino acid addition salts are salts with lysine, glycine, and phenylalanine.

The pharmaceutical composition according to the invention may further comprise at least one active compound, as defined above.

The pharmaceutical composition according to the invention can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures, or, for example, microcapsules, implants or rods.

The pharmaceutical composition can be prepared in a manner known per se to one of skill in the art. For this purpose, at least one compound according to the invention or a cyclodextrin salt thereof as defined above, one or more solid or liquid pharmaceutical excipients and, if desired, in combination with other pharmaceutical active compounds, are brought into a suitable administration form or dosage form which can then be used as a pharmaceutical in human medicine or veterinary medicine.

By means of non-limiting examples, such a formulation may be in a form suitable for oral administration, for parenteral administration (such as by intravenous, intramuscular, or subcutaneous injection, or intravenous infusion), for topical administration (including ocular), for administration by inhalation, by a skin patch, by an implant, by a suppository, etc. Such suitable administration forms—which may be solid, semi-solid, or liquid, depending on the manner of administration—as well as methods and carriers, diluents and excipients for use in the preparation thereof, will be clear to the skilled person; reference is made to for instance U.S. Pat. No. 6,372,778, U.S. Pat. No. 6,369,086, U.S. Pat. No. 6,369,087, and U.S. Pat. No. 6,372,733, as well as to the standard handbooks, such as the latest edition of Remington's Pharmaceutical Sciences.

As non-limiting examples, the active compound, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human medicine. For the production of pills, tablets, sugar-coated tablets and hard gelatin capsules it is possible to use, for example, lactose, starch, for example maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example of solutions for injection, or of emulsions or syrups are, for example, water, physiological sodium chloride solution, alcohols such as ethanol, glycerol, polyols, sucrose, invert sugar, glucose, mannitol, vegetable oils, etc. It is also possible to lyophilize the nucleic acid and/or the active compound and to use the resulting lyophilisates, for example, for preparing preparations for injection or infusion. Suitable carriers for microcapsules, implants or rods are, for example, copolymers of glycolic acid and lactic acid.

The pharmaceutical preparations can also contain additives, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

For an oral administration form, the compositions of the present invention can be mixed with suitable additives, such as excipients, stabilizers, or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate, and lactose and/or other excipients, binders, extenders, disintegrants, diluents, and lubricants known in the art.

The oral administration of a pharmaceutical composition comprising at least one compound according to the invention, or a pharmaceutically acceptable salt or ester and/or solvate thereof, is suitably accomplished by uniformly and intimately blending together a suitable amount of said compound in the form of a powder, optionally also including a finely divided solid carrier, and encapsulating the blend in, for example, a hard gelatin capsule. The solid carrier can include one or more substances, which act as binders, lubricants, disintegrating agents, coloring agents, and the like. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other pharmaceutically active substances (which may or may not lead to a synergistic effect with the compounds of the invention) and other substances that are commonly used in pharmaceutical formulations, such as lubricating agents, wetting agents, emulsifying, and suspending agents, dispersing agents, desintegrants, bulking agents, fillers, preserving agents, sweetening agents, flavoring agents, flow regulators, release agents, etc. The compositions may also be formulated so as to provide rapid, sustained, or delayed release of the active compound(s) contained therein, for example using liposomes or hydrophilic polymeric matrices based on natural gels or synthetic polymers.

Preferably, the present composition is administered in a GLP/GMP solvent, containing or not cyclobetadextrine and/or similar compounds.

The dosage or amount of compounds of the invention used, optionally in combination with one or more active compounds to be administered, depends on the individual case and is, as is customary, to be adapted to the individual circumstances to achieve an optimum effect. Thus, it depends on the nature and the severity of the disorder to be treated, and also on the sex, age, weight and individual responsiveness of the human or animal to be treated, on the efficacy and duration of action of the compounds used, on whether the therapy is acute or chronic or prophylactic, or on whether other active compounds are administered in addition to the agent(s) of the invention.

Without limitation, depending on the type and severity of the disease, a typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. A preferred dosage of the agent may be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks.

The pharmaceutical preparations of the invention are preferably in a unit dosage form, and may be suitably packaged, for example in a box, blister, vial, bottle, sachet, ampoule, or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of at least one compound of the invention, e.g. about 10, 25, 50, 100, 200, 300, or 400 mg per unit dosage.

In another embodiment, the invention provides a kit comprising a pharmaceutical composition according to the invention, and an active compound as defined herein, for simultaneous, separate or sequential administration to a subject in need thereof.

For these purposes, the compounds or the pharmaceutical compositions of the present invention may be administered orally, parenterally, i.e. including subcutaneous injections, intravenous, intramuscular, intrasternal injection, or infusion techniques, by inhalation spray, or rectally, in dosage unit formulations containing conventional non-toxic pharmaceutically acceptable carriers, adjuvants, and vehicles. At least one compound of the invention will generally be administered in an “effective amount”, by which is meant any amount of a compound of the Formula I or a cyclodextrin salt thereof as defined above above that, upon suitable administration, is sufficient to achieve the desired therapeutic or prophylactic effect in the individual to which it is administered. Usually, depending on the condition to be prevented or treated and the route of administration, such an effective amount will usually be between 0.01 to 1000 mg per kilogram body weight, more often between 0.1 and 500 mg, such as between 1 and 250 mg, for example about 5, 10, 20, 50, 100, 150, 200, or 250 mg, per kilogram body weight day of the patient per day, which may be administered as a single daily dose, divided over one or more daily doses, or essentially continuously, e.g. using a drip infusion. The amount(s) to be administered, the route of administration and the further treatment regimen may be determined by the treating clinician, depending on factors such as the age, gender and general condition of the patient and the nature and severity of the disease/symptoms to be treated.

In accordance with the method of the present invention, said pharmaceutical composition can be administered separately at different times during the course of therapy or concurrently in divided or single combination forms. The present invention is therefore to be understood as embracing all such regimes of simultaneous or alternating treatment and the term “administering” is to be interpreted accordingly.

Essentially, the primary modes of treatment of solid tumor cancers comprise surgery, radiation therapy, and chemotherapy, separately and in combination. The compounds according to the invention are suitable for use in combination with these medicinal techniques. The compounds of the invention may be useful in increasing the sensitivity of tumor cells to radiation in radiotherapy and also in potentiating or enhancing damage to tumors by chemotherapeutic agents. The compounds and their pharmaceutically acceptable salts and/or solvates may also be useful for sensitizing multidrug-resistant tumor cells. The compounds according to the invention are useful therapeutic compounds for administration in conjunction with DNA-damaging cytotoxic drugs or radiation used in radiotherapy to potentiate their effect.

In another embodiment of the method of the invention, the administration may be performed with food, e.g., a high-fat meal. The term “with food” means the consumption of a meal either during or no more than about one hour before or after administration of a pharmaceutical composition according to the invention.

Oral administration of a pharmaceutical composition comprising at least one compound according to the invention, or a pharmaceutically acceptable salt or ester and/or solvate thereof can also be accomplished by preparing capsules or tablets containing the desired amount of said compound, optionally blended with a solid carrier as described above. Compressed tablets containing the pharmaceutical composition of the invention can be prepared by uniformly and intimately mixing the active ingredient with a solid carrier such as described above to provide a mixture having the necessary compression properties, and then compacting the mixture in a suitable machine to the shape and size desired. Molded tablets maybe made by molding in a suitable machine, a mixture of powdered compound moistened with an inert liquid diluent.

When administered by nasal aerosol or inhalation, these compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions, or emulsions of the compounds of the invention or their physiologically tolerable salts in a pharmaceutically acceptable solvent, such as ethanol or water, or a mixture of such solvents. If required, the formulation can also additionally contain other pharmaceutical auxiliaries such as surfactants, emulsifiers and stabilizers as well as a propellant.

For subcutaneous or intravenous administration, the compound of the invention, if desired with the substances customary therefore such as solubilizers, emulsifiers, or further auxiliaries, are brought into solution, suspension, or emulsion. The compounds of the invention can also be lyophilized and the lyophilizates obtained used, for example, for the production of injection or infusion preparations. Suitable solvents are, for example, water, physiological saline solution, or alcohols, e.g. ethanol, propanol, glycerol, in addition also sugar solutions such as glucose or mannitol solutions, or alternatively mixtures of the various solvents mentioned. The injectable solutions or suspensions may be formulated according to known art, using suitable non-toxic, parenterally-acceptable diluents, or solvents, such as mannitol, 1,3-butanediol, water, Ringer's solution, or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, these formulations may be prepared by mixing the compounds according to the invention with a suitable non-irritating excipient, such as cocoa butter, synthetic glyceride esters, or polyethylene glycols, which are solid at ordinary temperatures, but liquidify and/or dissolve in the rectal cavity to release the drug.

The pharmaceutical compositions of this invention can be administered to humans in dosage ranges specific for each compound comprised in said compositions. The compounds comprised in said composition can be administered together or separately.

It will be understood, however, that specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

### EXAMPLES

The invention is illustrated by the following non-limiting examples

### Example 1

### Synthesis of the Compounds According to the Invention

### General

¹H NMR (300 M), spectra were recorded on a Bruker Avance® Spectrometer. The ¹H NMR chemical shifts were reported in parts par million (ppm) relative to the singlet at 7.26 ppm for chloroform in deuteriochloroform and the coupling constants are in Hz. The following abbreviations are used for spin multiplicity: s, singlet; d, doublet; t, triplet; q, quadruplet, qt, quintuplet; m, multiplet; b, broad. Routine thin layer chromatography (TLC) was performed on silica gel plates (Silicagel GF254® from VWR), column chromatography was performed on silica gel (spherical particle size 60-200 μm from MP Biomedicals). Solvents from Aldrich were used without further purification.

### 1. Synthesis of Intermediate I: 3-methoxy-4-[(methoxycarbonyl)oxy]benzoyl chloride

### 1.A. Synthesis of 3-Methoxy-4-[(methoxycarbonyl)oxy]benzoic acid

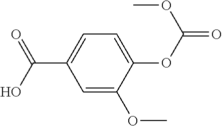

3-Methoxy-4-[(methoxycarbonyl)oxy]benzoic acid was synthesised according to the method described by K. Hallman (Tetrahedron: Asymmetry 10 (1999) 4037-4046): Vanillic acid (4 g, 23.8 mmoles) was dissolved in sodium hydroxide 0.5M (140 mL, 70 mmoles) in water at 0° C. under vigorous stirring. Methyl chloroformate (15 mL, 37.9 mmoles) was added over a period of 10-15 minutes. The reaction was allowed to warm to room temperature then stirred overnight and quenched by adding HCl 2 M until a pH of 3 was reached. A white precipitate was obtained, filtered off, washed with water and finally dried. Yield: 93% of white crystals.

For some products, the final deprotection didn't furnished the desired product so other protecting groups were tested, for example benzyl ether.

### 1.B. Synthesis of 3-methoxy-4-[(methoxycarbonyl)oxy]benzoyl chloride (I1)

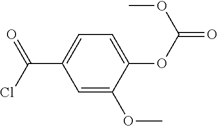

3-methoxy-4-(Methoxycarbonyloxy)benzoic acid (cf. A.1. 5 g, 22 mmoles) was dissolved under vigorous stirring in thionyl chloride (15 ml) cooled with a ice-bath at 0° C.

The reaction mixture was let to warm to room temperature and then refluxed for 30 minutes at 60° C. After this period, the reagent was evaporated under reduced pressure. The residue was dissolved in dichloromethane (15 mL) and the solvent was evaporated under reduced pressure. This operation was done two times.

Yield: 96% of white crystals.

### 2. Synthesis of Intermediate 2: 3-methoxy-4-benzyloxy-benzoyl chloride

### 2.A. Synthesis of 3-Methoxy-4-benzyloxy-benzoic acid

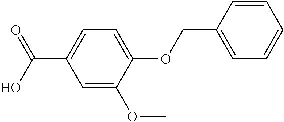

To a stirred solution of vanillic acid (5 g, 30 mmol) in THF (15 mL), a solution of NaOH (3 g) in water (37 mL) is added. The medium is cooled at 0° C. and a solution of benzyl chloride (4.1 mL, 34.8 mmol) is added. The medium is allowed to warm to room temperature and is then heated at 70° C. for 18 hours and then at 90° C. for 4 hours. After cooling to room temperature, the organic solvent is evaporated and the residual aqueous phase is acidified with 2M HCl. The precipitate is filtered and washed with cyclohexane to afford 4.8 g of white solid. Yield: 63%.

### 4-benzyloxy-3-methoxybenzoic acid

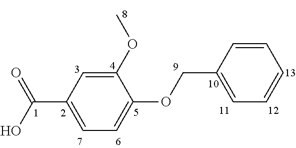

¹H NMR (DMSO): 12.68 (bs; 1H; COOH); 7.54 (dd; 2H; 7; ³J_{6,7}=8.4; ⁴J_{3,7}=2.1); 7.42 (m; 12H; 3, 11, 12&13); 7.14 (d; 2H; 6; ³J_{6,7}=8.4); 5.16 (s; 2H; 9); 3.80 (s; 6H; 8).

### 2.B. Synthesis of 3-methoxy-4-benzyloxy-benzoyl chloride

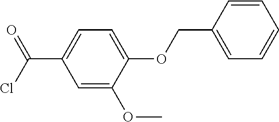

3-Methoxy-4-benzyloxy-benzoic acid (cf. A.2. 5 g, 22 mmoles) was dissolved under vigorous stirring in thionyl chloride (15 ml) cooled with a ice-bath at 0° C. The reaction mixture was let to warm to room temperature and then refluxed for 30 minutes at 60° C. After this period, the reagent was evaporated under reduced pressure. The residue was dissolved in dichloromethane (15 mL) and the solvent was evaporated under reduced pressure. This operation was done two times. Yield 82% of white solid

### 3. General Procedure for Esterification and Subsequent Hydrolysis

The diesters were synthesised according to the method described by Yu (Ang. Chem. Int. Ed. 46(6) 881-3 (2007)). Following synthesis, hydrolysis was performed. The complete general reaction is displayed in schemes 1 and 2.

### 3.A. Esterification:

Each of the different diols as exemplified below (9.5 mmoles), pyridine (4 mL, 49.5 mmol) and dichloromethane (10 mL) were added to either 3-methoxy-4-[(methoxycarbonyl)oxy]benzoyl chloride (cf. I1) or 3-methoxy-4-benzyloxy-benzoyl chloride (cf. I2) (each 21 mmol). The reaction mixture was refluxed for 10 minutes then stirred at room temperature overnight and quenched by adding HCl 2 M until a pH of 3 was reached. The organic layer was dried over Na2SO4 anhydrous and concentrated under vacuum.

### 3.B. Hydrolysis:

3.B.1. Hydrolysis of the methoxycarbonyl group of the di-esters (cf. Scheme 1) was performed by adding THF (30 mL) to a stirred suspension of the diester (4 mmol) in NH4OH (17 mL). The reaction mixture was stirred at room temperature for 100 minutes. The reaction was stopped by adding carefully concentrated HCl to reach pH 3. Ethyl acetate was added and, after shaking, the organic layer was dried over anhydrous Na2SO4 and concentrated under vacuum.

3.B.2. For hydrolysis of the diesters having the benzyloxy protective group as outlined in Scheme 2, the following protocol was followed: Pd on carbon 10% was added (0.4 mmol) to a stirred solution of the diester (4 mmol) in MeOH (17 mL). The reaction mixture was stirred at room temperature overnight under hydrogen (1 atm). The medium was filtered through celite and MeOH was evaporated under vacuum. The crude product was chromatographed only if necessary.

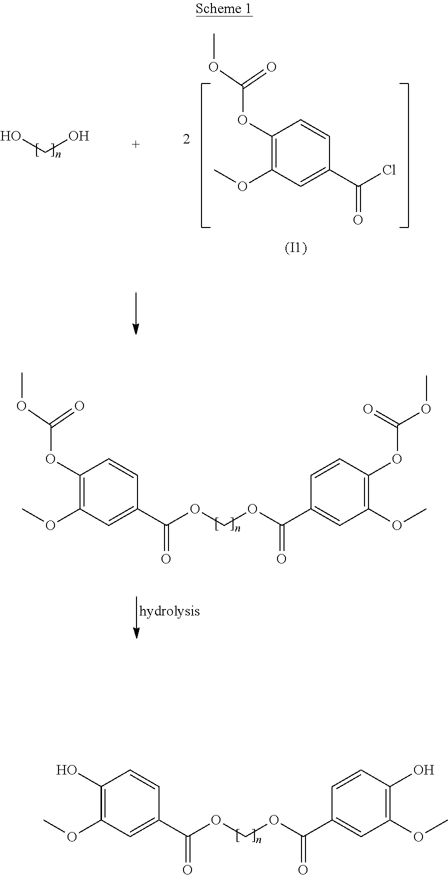

wherein n is an integer selected from 1-12

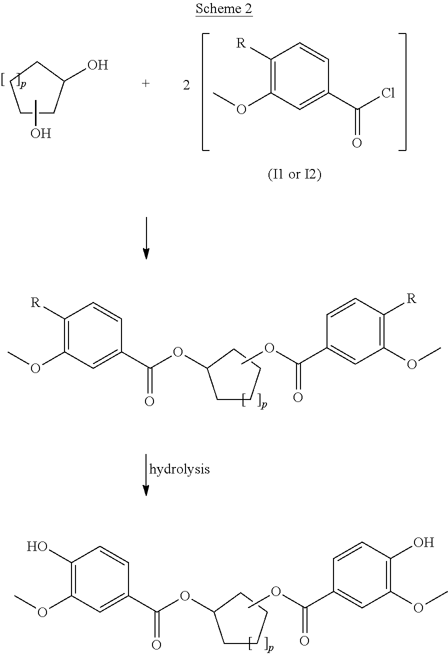

wherein p is an integer of 0, 1, 2, or 3
wherein R is a protective group needed is selected from:

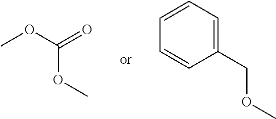

depending on whether I1 or I2 is used

### 4. General Protocol for the Synthesis of the Compounds of the Invention

### 4.A. Synthesis of compound DLT1: Ethane-1,2-diyl bis-(4-hydroxy-3-methoxybenzoate)

Following Scheme 1 above, Ethane-1,2-diol was reacted with I1, as outlined in point 3.A. resulting in Ethane-1,2-diyl bis-[3-methoxy-4-[(methoxycarbonyl)oxy]benzoate] (n=2): White solid; yield 70%

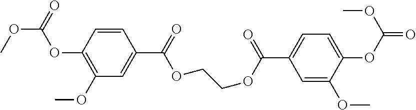

Following hydrolysis as outlined in point 3.B.1, Ethane-1,2-diyl bis-(4-hydroxy-3-methoxybenzoate) (working name DLT1) was obtained: Amber solid; yield 49%; ¹H-NMR: δ (CDCl₃): 7.67-7.647 (2H, dd), 7.55-7.54 (2H, d), 6.94-6.92 (2H, d), 6.04 (2H, s), 4.63 (4H, s), 3.92 (6H, s).

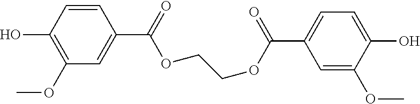

### 4.B. Synthesis of Compound DLT2

Following Scheme 1 above, Propane-1,3-diol was reacted with I1, as outlined in point 3.A. resulting in Propane-1,3-diyl bis-[3-methoxy-4-[(methoxycarbonyl)oxy]benzoate] (n=3): White powder; yield 70%

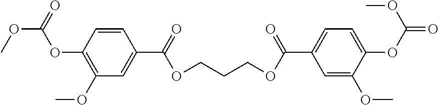

Following hydrolysis as outlined in point 3.B.1, Propane-1,3-diyl bis-(4-hydroxy-3-methoxybenzoate) (working name DLT2) was obtained: White powder; yield 56%; ¹H-NMR: δ (CDCl₃): 7.66-7.63 (2H, dd), 7.53-7.52 (2H, d), 6.93-6.91 (2H, d), 4.50 (4H, t, m), 3.90 (6H, s), 3.76 (2H, s), 2.26 (2H, m).

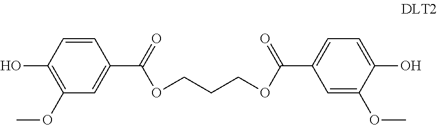

### 4.C. Synthesis of Compound DLT10

Following Scheme 1 above, Butane-1,4-diol was reacted with I1, resulting in Butane-1,4-diyl bis-[3-methoxy-4-[(methoxycarbonyl)oxy]benzoate] (n=4): White powder; yield 65%

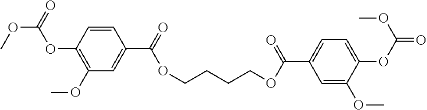

Following hydrolysis as outlined in point 3.B.1, Butane-1,4-diyl bis-(4-hydroxy-3-methoxybenzoate) (working name DLT10) was obtained: White powder; yield 60%; ¹H-NMR: δ (CDCl₃): 7.67-7.64 (2H, dd), 7.55-7.54 (2H, d), 6.94-6.92 (2H, d), 6.23 (2H, bs), 4.37 (2H, t), 3.95 (6H, s), 1.90 (4H, t, m).

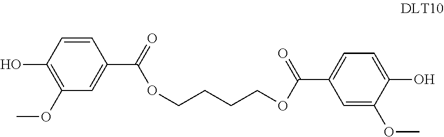

### 4.D. Synthesis of Compound DLT 3

Following Scheme 1 above, Pentane-1,5-diol was reacted with I1, resulting in Pentane-1,5-diyl bis-[3-methoxy-4-[(methoxycarbonyl)oxy]benzoate] (n=5): White powder; yield 70%

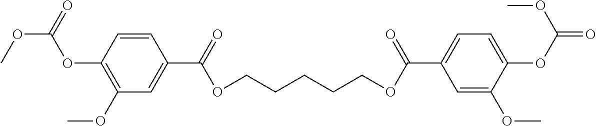

Following hydrolysis as outlined in point 3.B.1, Pentane-1,5-diyl bis-(4-hydroxy-3-methoxybenzoate) (working name DLT3) was obtained: White powder; yield 77%; ¹H-NMR: δ (CDCl₃): 7.67-7.647 (2H, d), 7.55-7.54 (2H, d), 6.94-6.92 (2H, d), 6.23 (2H, s), 4.37 (4H, t), 3.95 (6H, s), 1.86 (4H, m), 1.55 (2H, m)

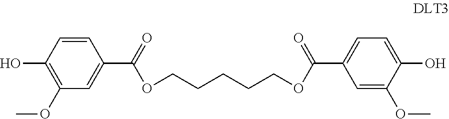

### 4.E. Synthesis of Compound DLT 11

Following Scheme 1 above, Hexane-1,6-diol was reacted with I1, resulting in Hexane-1,6-diyl bis-[3-methoxy-4-[(methoxycarbonyl)oxy]benzoate] (n=6): White solid; yield 60%

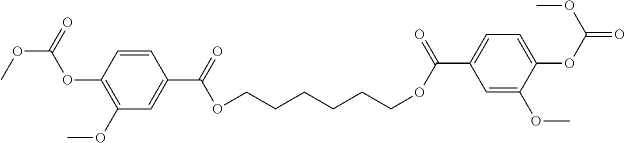

Following hydrolysis as outlined in point 3.B.1, Hexane-1,6-diyl bis-(4-hydroxy-3-methoxybenzoate) (working name DLT11) was obtained: White solid; yield 50%; ¹H-NMR: δ (CDCl₃): 7.67-7.64 (2H, dd), 7.55-7.54 (2H, d), 6.94-6.92 (2H, d), 6.23 (2H, s), 4.29 (4H, t), 3.91 (6H, s), 1.85 (4H, m) 1.52 (4H, t)

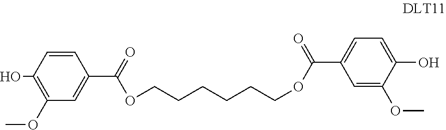

### 4.F. Synthesis of Compound DLT 4

Following Scheme 2 above, trans-cyclohexane-1,2-diol was reacted with I1, resulting in trans-cyclohexane-1,2-diyl bis-[3-methoxy-4 [(methoxycarbonyl)oxy]-benzoate]: Beige syrup; yield 70%

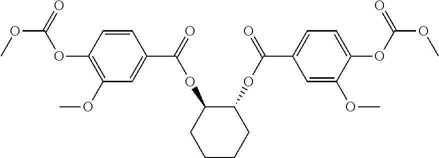

Following hydrolysis as outlined in point 3.B.1, trans-cyclohexane-1,2-diyl bis-(4-hydroxy-3-methoxybenzoate) (working name DLT4) was obtained: From carbonate: white solid; yield 39%; ¹H-NMR: δ (CDCl₃): 7.60-7.57 (2H, dd), 7.48-7.47 (2H, d), 6.90-6.87 (2H, d), 5.16 (2H, m), 3.85 (6H, s), 2.22 (2H, m), 1.81 (2H, m), 1.60-1.41 (4H, m).

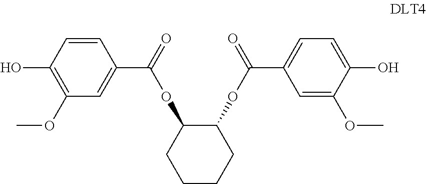

The pure trans enantiomers were also synthesized starting from enantiopur cyclohexane diols: DLT7 (S,S); yield 38% and DLT8 (R,R); yield 39%.

### 4.G. Synthesis of Compound DLT9

Following Scheme 2 above, cis-cyclohexane-1,2-diol was reacted with I1, resulting in cis-cyclohexane-1,2-diyl bis-[3-methoxy-4 [(methoxycarbonyl)oxy]-benzoate] or [(1R,2S)-2-(3-methoxy-4-methoxycarbonyloxy-benzoyl)oxycyclohexyl]3-methoxy-4-methoxy-carbonyloxy-benzoate:

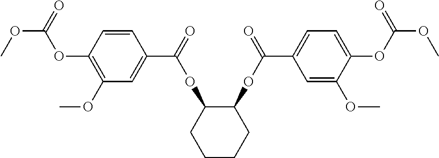

Following hydrolysis as outlined in point 3.B.1, cis-cyclohexane-1,2-diyl bis-(4-hydroxy-3-methoxybenzoate) (working name DLT9) was obtained: white solid; yield 30%; ¹H-NMR: δ (CDCl₃): 7.61-7.58 (2H, dd), 7.49-7.48 (2H, d), 6.89-6.86 (2H, d), 3.81 (6H, s), 4.55 (2H, s), 2.19 (2H, m), 1.82 (2H, m), 1.44 (4H, m).

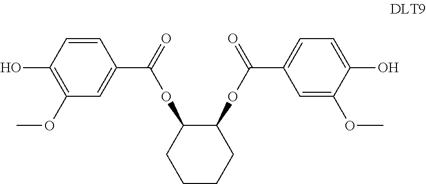

### 4.H. Synthesis of Compound DLT5

Following Scheme 2 above, cis- and trans-cyclohexane-1,3-diol was reacted with 12, resulting in cis- and trans-cyclohexane-1,3-diyl bis-[3-methoxy-4 benzyloxy]benzoate] Starting from a mixture of cis/trans 1,3 cyclohexane diol, the isomers seems to be separable by chromatography but so far, we obtained the cis isomer as a 80/20 mixture and the trans isomer as a mixture 60/40 mixture. Respective yields: 20% and 15%. White solids.

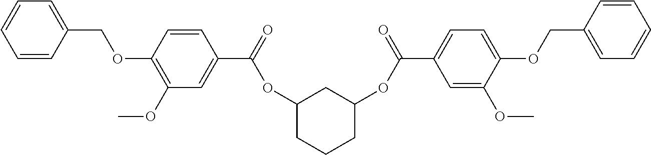

Following hydrolysis as outlined in point 3.B.2, a racemic mixture of cis and trans-Cyclohexane-1,3-diol di-(4-hydroxy-3-methoxybenzoate) (DLT5) was obtained: white solid;

First experiment yield=57%; ¹H-NMR: δ (CDCl₃): 7.63-7.59 (2H, dd), 7.52-7.49 (2H, d), 6.92-6.85 (2H, d), 5.16 (2H, m), 3.82 (6H, s), 2.24 (2H, m), 1.85 (4H, m), 1.50 (2H, m).

Second experiment yield=24%; Rf=0.3 (Cyclohexane/AcOEt 8/2). Only the peaks of the major product are described

¹H NMR (DMSO): 7.48 to 7.44 (m; 4H; 3&7); 6.84 (d; 2H; 6; ³J_{4,7}=8.1); 4.99 (bs; 2H; 10); 3.80 (s; 6H; 8); 2.38 to 1.88 (m; 8H; 9, 11&12).

¹³C NMR (DMSO): 164.6 (1); 151.2 (5); 147.0 (4); 123.1 (7); 120.4 (2); 114.7 (6); 112.2 (3); 69.8 (10); 55.3 (8); 36.0 (9); 29.9 (11); 18.7 (12).

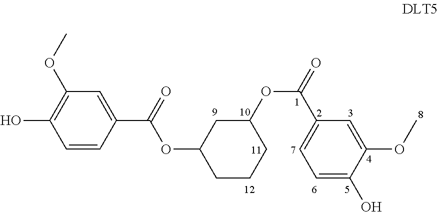

### 5. An Other General Procedure for the Three Step Synthesis Using Benzyl Ether as Phenol Protecting Group

The diol (100 to 200 mg), the DMAP (2.1 eq.) and the protected vanillic acid (3-Methoxy-4-benzyloxy-benzoic acid (cf. A.2.) 2.5 eq.) are weight in the reactor. Toluene is added (100 mL for 100 mg of diol) and then the DCC (2.3 eq.). The medium is stirred at RT for 3 to 4 days. The solvent is evaporated to dryness and the medium is directly purified by silica gel chromatography using the eluent given for the Rf.

The dibenzylated compound (200 to 500 mg) is weight in the reactor. MeOH is added (20 mL for 100 mg) and the medium is cooled by a water-ice bath before addition of the 10% Palladium on carbon catalyst (same weight as the dibenzylated product). The medium is then placed under hydrogen atmosphere and stirred at RT overnight. The medium is filtered through silica gel and then chromatographed if necessary.

Yield=63%

### 4-benzyloxy-3-methoxybenzoic acid

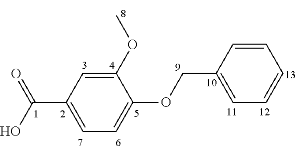

¹H NMR (DMSO): 12.68 (bs; 1H; COOH); 7.54 (dd; 2H; 7; ³J_{6,7}=8.4; ⁴J_{3,7}=2.1); 7.42 (m; 12H; 3, 11, 12&13); 7.14 (d; 2H; 6; ³J_{6,7}=8.4); 5.16 (s; 2H; 9); 3.80 (s; 6H; 8).

### 5. A. Synthesis of Compound DLT8:

### Trans-R,R-1,2-cyclohexane diyl bis(4-benzyloxy-3-methoxybenzoate)

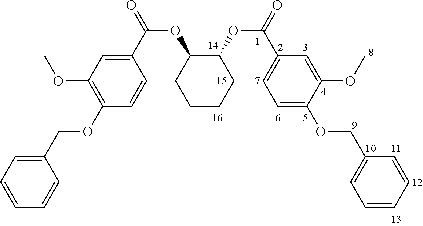

Yield=48%; Rf=0.25 (Petroleum ether/AcOEt 8/2)

¹H NMR (DMSO): 7.50 (dd; 2H; 7; ³J_{6,7}=8.5; ⁴J_{3,7}=1.8); 7.40 (m; 12H; 3, 14, 15&16); 7.08 (d; 2H; 6; ³J_{6,7}=8.5); 5.10 (bs; 6H; 8&12); 3.75 (s; 6H; 11); 2.10 (m; 2H; 9a); 1.72 to 1.42 (m; 6H; 2b, 3a&3b).

¹³C NMR (DMSO): 164.9 (1); 151.9 (5); 148.5 (4); 136.3 (10); 128.4/127.8 (11&12); 127.9 (13); 122.8 (7); 121.9 (2); 112.4 (6); 111.7 (3); 74.1 (9); 69.8 (14); 55.54 (8); 29.7 (15); 23.0 (16).

### Trans-R,R-1,2-cyclohexane diyl bis(4-hydroxy-3-methoxybenzoate) (DLT8)

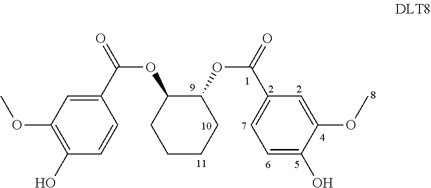

Yield=81%; Rf=0.25 (Petroleum ether/AcOEt 8/2)

¹H NMR (DMSO): 7.36 (m; 4H; 7&3); 6.81 (d; 2H; 6; ³J_{6,7}=8.1); 5.08 (bs; 2H; 9); 2.13 to 2.10 (m; 2H; 10a); 1.78 to 1.48 (m; 6H; 10b&11).

¹³C NMR (DMSO): 164.9 (1); 151.9 (5); 148.5 (4); 136.3 (10); 128.4/127.8 (11&12); 127.9 (13); 122.8 (7); 121.9 (2); 112.4 (6); 111.7 (3); 74.1 (9); 69.8 (14); 55.54 (8); 29.7 (15); 23.0 (16).

### 5.B. Synthesis of Compound DLT7:

### Trans-S,S-1,2-cyclohexane diyl bis(4-benzyloxy-3-methoxybenzoate)

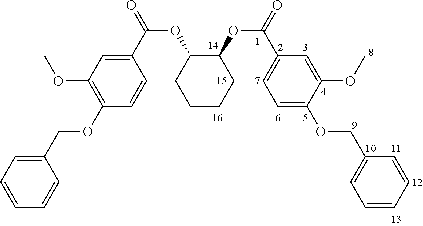

Yield=29%; Rf=0.25 (Petroleum ether/AcOEt 8/2)

¹H NMR (DMSO): 7.50 (dd; 2H; 7; ³J_{6,7}=8.5; ⁴J_{3,7}=1.8); 7.40 (m; 12H; 3, 14, 15&16); 7.08 (d; 2H; 6; ³J_{6,7}=8.5); 5.10 (bs; 6H; 8&12); 3.75 (s; 6H; 11); 2.10 (m; 2H; 9a); 1.72 to 1.42 (m; 6H; 2b, 3a&3b).

¹³C NMR (DMSO): 164.9 (1); 151.9 (5); 148.5 (4); 136.3 (10); 128.4/127.8 (11&12); 127.9 (13); 122.8 (7); 121.9 (2); 112.4 (6); 111.7 (3); 74.1 (9); 69.8 (14); 55.54 (8); 29.7 (15); 23.0 (16).

### Trans-S,S-1,2-cyclohexane diyl bis(4-hydroxy-3-methoxybenzoate) (DLT7)

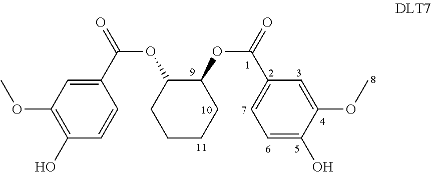

Yield=71%; Rf=0.25 (Petroleum ether/AcOEt 8/2)

¹H NMR (DMSO): 7.36 (m; 4H; 7&3); 6.81 (d; 2H; 6; ³J_{6,7}=8.1); 5.08 (bs; 2H; 9); 2.13 to 2.10 (m; 2H; 10a); 1.78 to 1.48 (m; 6H; 10b&11).

¹³C NMR (DMSO): 164.9 (1); 151.9 (5); 148.5 (4); 136.3 (10); 128.4/127.8 (11&12); 127.9 (13); 122.8 (7); 121.9 (2); 112.4 (6); 111.7 (3); 74.1 (9); 69.8 (14); 55.54 (8); 29.7 (15); 23.0 (16).

### 5.C. Synthesis of Compound DLT9:

### Cis-1,2-Cyclohexane diyl bis(4-benzyloxy-3-methoxybenzoate)

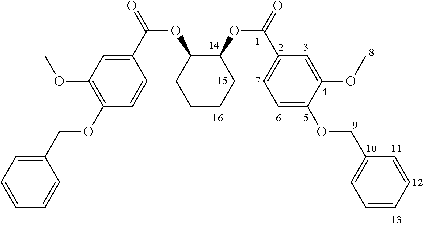

Yield=24%; Rf=0.3 (Cyclohexane/AcOEt 8/2)

¹H NMR (DMSO): 7.56 (dd; 2H; 7; ³J_{6,7}=8.5; ⁴J_{3,7}=2.1); 7.40 (m; 12H; 3, 11, 12&13); 7.12 (d; 2H; 6; ³J_{6,7}=8.5); 5.27 (bs; 2H; 14); 5.16 (s; 4H; 9); 3.66 (s; 6H; 8); 1.92 to 1.51 (m; 8H; 15&16).

¹³C NMR (DMSO): 164.9 (1); 151.9 (5); 148.5 (4); 136.3 (10); 128.4/127.8 (11&12); 127.9 (13); 122.8 (7); 121.9 (2); 112.4 (6); 111.7 (3); 74.1 (9); 69.8 (14); 55.54 (8); 29.7 (15); 23.0 (16).

### cis-1,2-cyclohexane diyl bis(4-hydroxy-3-methoxybenzoate) (DLT9)

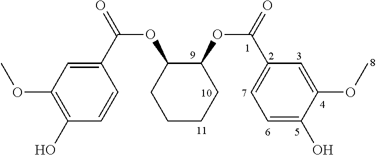

Yield=100%; Rf=0.3 (Cyclohexane/AcOEt 6/4)

¹H NMR (DMSO): 7.50 (dd; 2H; 7; ³J_{6,7}=8.5; ⁴J_{3,7}=1.8); 7.40 (m; 12H; 3, 14, 15&16); 7.08 (d; 2H; 6; ³J_{6,7}=8.5); 5.10 (bs; 6H; 8&12); 3.75 (s; 6H; 11); 2.10 (m; 2H; 9a); 1.72 to 1.42 (m; 6H; 2b, 3a&3b).

¹³C NMR (DMSO): 164.9 (1); 151.9 (5); 148.5 (4); 136.3 (10); 128.4/127.8 (11&12); 127.9 (13); 122.8 (7); 121.9 (2); 112.4 (6); 111.7 (3); 74.1 (9); 69.8 (14); 55.54 (8); 29.7 (15); 23.0 (16).

### 6. General Procedures for the Selective Alkylation of Vanillic Acid with Dibrominated Compounds

The dialkylating agent (100 to 300 mg), vanillic acid (2.2 eq.) and NaHCO₃ (2.2 eq.) are weight in the reactor. DMF is added (10 mL for 100 mg) and the medium is heated at 110° C. for 12 h. The medium is partitioned between water and AcOEt, the aqueous phase is extracted three times, the organic phases are dried over Na₂SO₄ and concentrated under vacuum. The crude product obtained is then purified by silica gel chromatography using the eluant specified for the Rf.

### 6.A. Synthesis of Compound DLT10:

### 1,4-Butane diyl bis(4-hydroxy-3-methoxybenzoate) (or 4-(4-hydroxy-3-methoxy-benzoyl)oxybutyl 4-hydroxy-3-methoxy-benzoate; methanol) (working name DLT10)

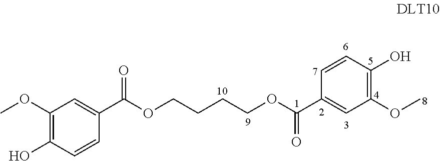

Yield=42%

¹H NMR (DMSO): 9.97 (bs; 2H; OH); 7.44 (m; 4H; 3&7); 6.85 (d; 2H; 6; ³J_{6,7}=8.4); 4.28 (bs; 4H; 9); 3.79 (s; 6H; 8); 1.82 (bs; 4H; 10).

¹³C NMR (DMSO): 165.6 (1); 151.4 (5); 147.3 (4); 123.3 (7); 120.6 (2); 115.1 (6); 112.4 (3); 63.9 (9); 55.5 (8); 25.1 (10).

### 6.B. Synthesis of Compound DLT11:

### 1,6-Hexane diyl bis(4-hydroxy-3-methoxybenzoate) (or 6-(4-hydroxy-3-methoxy-benzoyl)oxyhexyl 4-hydroxy-3-methoxy-benzoate; methanol) (working name DLT11)

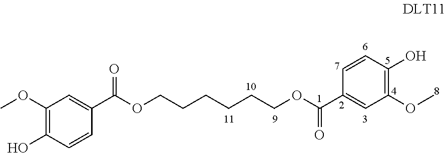

Yield=44%

¹H NMR (DMSO): 9.98 (bs; 2H; OH); 7.44 (m; 4H; 3&7); 6.85 (d; 2H; 6; ³J_{6,7}=8.1); 4.20 (t; 4H; 9; ³J_{6,7}=6.4); 3.79 (s; 6H; 8); 1.70 (bs; 4H; 10); 1.44 (bs; 4H; 11).

¹³C NMR (DMSO): 165.6 (1); 151.4 (5); 147.3 (4); 123.3 (7); 120.7 (2); 115.2 (6); 112.4 (3); 64.1 (9); 55.6 (8); 28.2 (10); 25.2 (11).

### 6.C. Synthesis of Compound DLT12:

### 1,7-Heptane diyl bis(4-hydroxy-3-methoxybenzoate) (or 7-(4-hydroxy-3-methoxy-benzoyl)oxyheptyl 4-hydroxy-3-methoxy-benzoate; methanol) (working name DLT12)

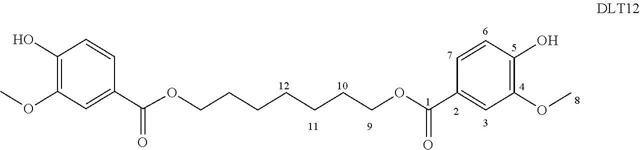

Yield=40%

¹H NMR (DMSO): 9.94 (bs; 2H; OH); 7.44 (m; 4H; 3&7); 6.85 (d; 2H; 6; ³J_{6,7}=8.1); 4.20 (t; 4H; 9; ³J_{6,7}=6.4); 3.80 (s; 6H; 8); 1.68 (bs; 4H; 10); 1.39 (bs; 6H; 11&12).

¹³C NMR (DMSO): 165.5 (1); 151.4 (5); 147.3 (4); 123.2 (7); 120.6 (2); 115.1 (6); 112.4 (3); 64.1 (9); 55.5 (8); 28.2 (12); 28.1 (10); 25.3 (11).

### 6.D. Synthesis of Compound DLT13:

### 1,8-Octane diyl bis(4-hydroxy-3-methoxybenzoate) (or 7-(4-hydroxy-3-methoxy-benzoyl)oxyheptyl 4-hydroxy-3-methoxy-benzoate; methanol) (working name DLT13)

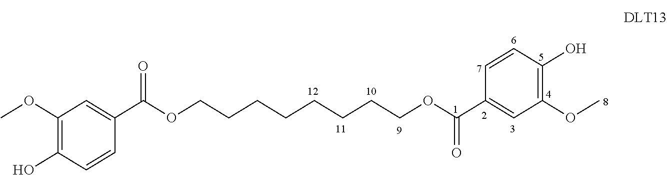

Yield=41%

¹H NMR (CDCl₃): 7.63 (dd; 2H; 7; ³J_{6,7}=8.1; ⁴J_{3,7}=1.8); 7.55 (d; 2H; 3; ⁴J_{3,7}=1.8); 6.93 (d; 2H; 6; ³J_{6,7}=8.1); 4.28 (t; 4H; 9; ³J_{9,10}=6.7); 3.93 (s; 6H; 8); 1.76 (m; 4H; 10); 1.40 (bs; 8H; 11&12).

¹³C NMR (CDCl₃): 166.5 (1); 149.9 (5); 146.1 (4); 124.0 (7); 122.6 (2); 114.0 (6); 111.7 (3); 64.9 (9); 56.1 (8); 29.2/28.2 (10&12); 26.0 (11).

### 6.E. Synthesis of Compound DLT24:

### α,α′-o-Xylene diyl bis(4-hydroxy-3-methoxybenzoate) (or [2-[(4-hydroxy-3-methoxy-benzoyl)oxymethyl]phenyl]methyl 4-hydroxy-3-methoxy-benzoate; methanol)

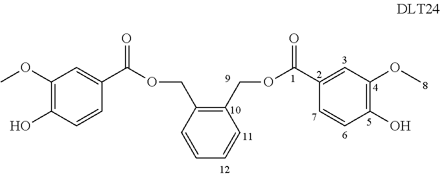

Yield=43; Rf=0.17 (Petroleum ether/AcOEt 5/5)

¹H NMR (CDCl₃+1 drop of DMSO-d6): 7.58 (dd; 2H; 7; ³J_{6,7}=8.4; ⁴J_{3,7}=2.1); 7.51 (m; 4H; 3&11); 7.37 (m; 2H; 12); 6.85 (d; 2H; 6; ³J_{6,7}=8.4); 5.49 (s; 4H; 9); 3.87 (s; 6H; 8).

¹³C NMR (CDCl₃+1 drop of DMSO-d6): 165.9 (1); 150.6 (5); 146.6 (4); 134.7 (10); 129.6/128.5 (11&12); 124.0 (7); 121.3 (2); 114.3 (6); 112.0 (3); 64.0 (9); 55.8 (8).

Exemplary compounds based on the general formulas (I), (II), (III), (IV), (V), and (VI) that can be made through similar synthesis routes as the ones exemplified above are displayed in Table 1. For some of these examples, we considered only symmetrical compounds: R1=R4, R2=R5 and R3=R6. However, it is of course possible to synthesise dissymmetrical compounds. In cases were needed, the commercial diols are either available as pure enantiomers, or it is possible to separate the two enantiomers of the final product by crystallization of diastereomer salts with a chiral base.

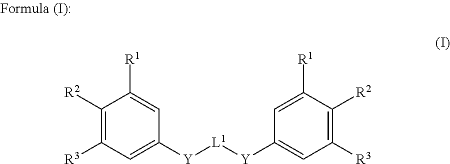

wherein R¹⁻³=H, OH, C₁₋₈ alkoxyalylene, OMe, halogen;
wherein Y is selected from the group comprising COO, tetrazole, OCO, OCOO, CONR⁴, NR⁴CO, OCONR⁴, NR⁴COO, NR⁴CONR⁴;
wherein R⁴═H, O₁₋₄ Alkyl;
wherein L¹=C₁₋₈ alkylene, preferably C₅₋₁₀; or (CH₂)ₙ, wherein n is an integer selected from 2-10 or

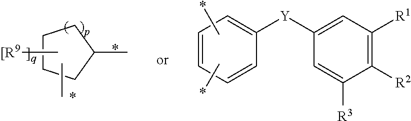

(* The asterisk is used herein to indicate the point at which a mono- or bivalent radical depicted is connected to the structure to which it relates and of which the radical forms part), wherein p is an integer selected from 0, 1, 2, or 3
and, wherein each group can optionally be substituted with one or two substances selected form the group comprising CO₂H, vanillic acid, alkyloxycarbonyl, . . .

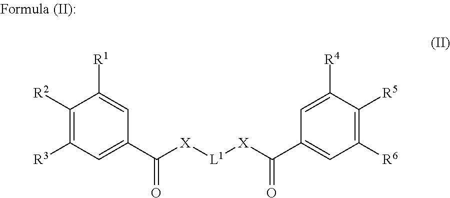

wherein X is selected from the group comprising O, NH, N—C₁₋₆alkyl;
wherein each R¹, R², R³, R⁴, R⁵, R⁶ is independently selected from the group comprising H, OH, C₁₋₆alkoxyC₁₋₆alkyl, C₁₋₆alkoxy, and halogen; L¹ is a group selected from C₁₋₈alkylene or

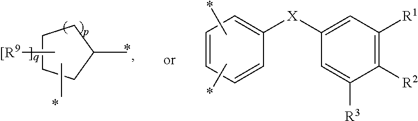

(* The asterisk is used herein to indicate the point at which a mono- or bivalent radical depicted is connected to the structure to which it relates and of which the radical forms part), each group being optionally substituted with one, two or three substituents each independently selected form the group comprising C₁₋₆alkyl, CO₂H, vanillic acid, amine, and C₁₋₆alkyloxycarbonyl, wherein p is an integer selected from 0, 1, 2, or 3;
R⁹ is selected from the group comprising OH, CO₂H, NH₂ and q is an integer selected from 0, 1, 2, or 3; or stereoisomeric forms thereof.

Original structures envisaged according to exactly the same synthetic strategy using preferentially the benzyle protecting group for the phenol.

Exemplary compounds based on Formula I:

### 6-(3,4-dimethoxybenzoyl)oxyhexyl 3,4-dimethoxybenzoate (DLT 26)

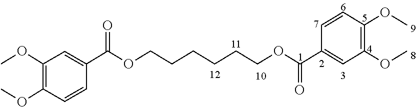

Yield=72%; R_{f}=0.2 (CH₂Cl₂/AcOEt 97/3). ¹H NMR (DMSO+a few drops CDCl₃): 7.55 (dd; 2H; 7; ³J_{3,7}=8.4; ⁴J_{3,7}=1.8); 6.98 (d; 2H; 6; ³J_{6,7}=8.4); 4.23 (t; 4H; 10; ³J_{6,7}=6.4); 3.82/3.79 (2 s; 12H; 8&9); 1.73 (bs; 4H; 11); 1.47 (bs; 4H; 12). ¹³C NMR (DMSO+a few drops CDCl₃): 165.3 (1); 152.7 (5); 148.2 (4); 122.9/121.9 (2&7); 111.5/110.7 (3&6); 64.0 (10); 55.5/55.3 (8&9); 28.0 (11); 25.1 (12).

### 1,4-oxybut-2-enyl-bis(4-hydroxy-3-methoxybenzoate) (or [(E)-4-(4-hydroxy-3-methoxy-benzoyl)oxybut-2-enyl]-4-hydroxy-3-methoxy-benzoate; methanol) (DLT 27)

### Mixture of Cis and Trans

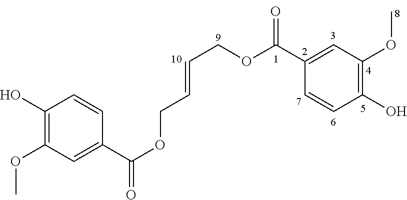

Only the peaks of the major product (probably the trans) are described. ¹H NMR (DMSO): 9.98 (bs; 2H; OH); 7.49 (dd; 2H; 7; ³J_{6,7}=8.1; ⁴J_{3,7}=1.8); 7.44 (d; 2H; 3; ⁴J_{3,7}=1.8); 6.87 (d; 2H; 6; ³J_{6,7}=8.1); 6.03 (bs; 2H; 6); 4.79 (bs; 4H; 9); 3.81 (s; 6H; 8).

¹³C NMR (DMSO): 165.1 (1); 151.5 (5); 147.3 (4); 127.9 (10); 123.4 (7); 120.2 (2); 115.1 (6); 112.4 (3); 63.6 (9); 55.5 (8).

### 1,4-oxybut-2-ynyl bis(4-hydroxy-3-methoxybenzoate) (DLT28)

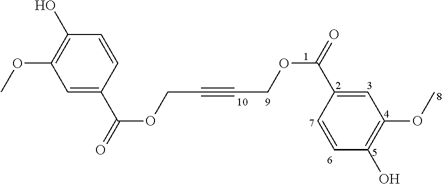

¹H NMR (DMSO): 10.05 (bs; 2H; OH); 7.48 (dd; 2H; 7; ³J_{6,7}=8.2; ⁴J_{3,7}=1.4); 7.43 (d; 2H; 3; ⁴J_{3,7}=1.4); 6.89 (d; 2H; 6; ³J_{6,7}=8.2); 5.99 (s; 4H; 9); 3.82 (s; 6H; 8).¹³C NMR (DMSO): 164.7 (1); 151.8 (5); 147.4 (4); 123.7 (7); 119.6 (2); 115.2 (6); 112.5 (3); 81.3 (10); 55.5 (8); 51.9 (9).

### 6-(3-hydroxy-4-methoxy-benzoyl)oxyhexyl 3-hydroxy-4-methoxy-benzoate (DLT29)

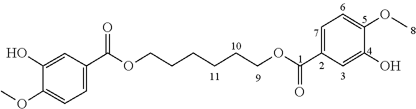

Yield=29%; R_{f}=0.2 (Cyclohexane/AcOEt 6/4). ¹H NMR (DMSO): 9.41 (bs; 2H; OH); 7.42 (dd; 2H; 7; ³J_{6,7}=8.4; ⁴J_{3,7}=2.1); 7.37 (d; 2H; 3; ³J_{3,7}=2.1); 6.98 (d; 2H; 6; ³J_{6,7}=8.4); 4.20 (t; 4H; 9; ³J_{6,7}=6.4); 3.82 (s; 6H; 8); 1.70 (bs; 4H; 10); 1.44 (bs; 4H; 11).

¹³C NMR (DMSO): 165.5 (1); 151.8 (5); 146.2 (4); 122.1/121.4 (2&7); 115.6 (6); 111.4 (3); 64.1 (9); 55.6 (8); 28.1 (10); 25.2 (11).

### [3-[(4-hydroxy-3-methoxy-benzoyl)oxymethyl]phenyl]methyl 4-hydroxy-3-methoxy-benzoate (DLT25)

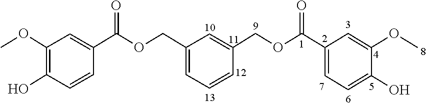

Yield=43% (256 mg); R_{f}=0.17 (Cyclohexane/AcOEt 7/3); HPLC purity:

¹H NMR (DMSO): 9.99 (bs; 2H; OH); 7.51 to 7.41 (m; 8H; 3,7, 10, 12&13); 6.85 (d; 2H; 6; ³J_{8,7}=8.4); 5.31 (s; 4H; 9); 3.79 (s; 6H; 8). ¹³C NMR (DMSO): 165.0 (1); 150.6 (5); 146.6 (4); 136.5 (11); 128.4/127.1/126.7 (10, 12&13); 123.2 (7); 120.0 (2); 114.9 (6); 112.2 (3); 65.1 (9); 55.2 (8).

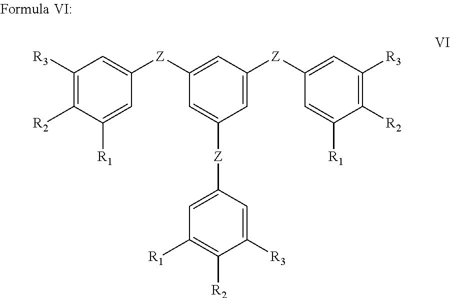

wherein Z is selected from the group comprising COCH₂CO, COCH₂CH₂, CH₂CH₂CO, CH₂COCH₂, COOCH₂, CONHCH₂, CON—C₁₋₆alkylCH₂, CONHCO, CON—C₁₋₆alkylCO, CH₂NHCH₂, CH₂N—C₁₋₆alkylCH₂, CH₂OCO, CH₂NHCO, CH₂N(C₁₋₆alkyl)CO, CH₂OCH₂, CH₂SCH₂, SO₂OCH₂, SO₂NHCH₂, SO₂N—C₁₋₆alkylCH₂;
and wherein R¹⁻³ can be each independently of each other ═H, OH, Halogen, O₁₋₈ alkoxyalkylene, OMe Ac, OAc, C₁₋₈ alkyl, NO₂;
and wherein two contiguous substituents among R¹⁻³ can be together a dioxole.

Exemplary compounds of Formula VI that have already been synthesized by a single step as depicted below are as follows:

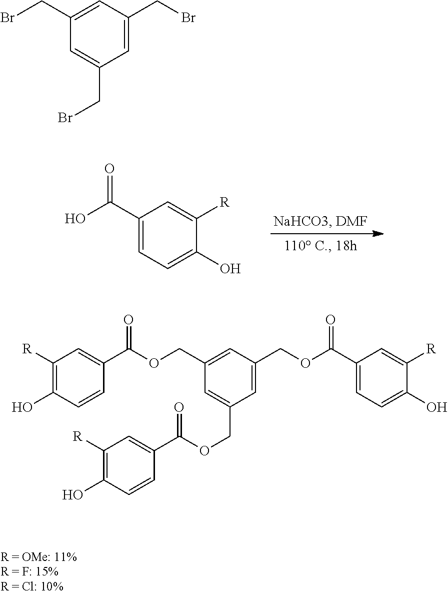

### [3,5-bis[(4-hydroxy-3-methoxy-benzoyl)oxymethyl]phenyl]methyl 4-hydroxy-3-methoxy-benzoate. DLT95

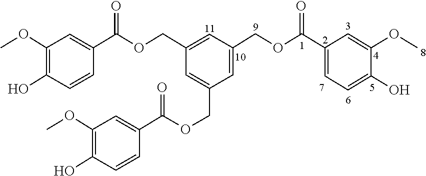

Yield=11%, R_{f}=0.23 (Cyclohexane/AcOEt: 5/5), then precipitation from CH₂Cl₂. RP-HPLC: purity=100% (254 nm), t_{R}=4.04 min; ¹H NMR (DMSO-d6): δ 9.99 (bs, 3H, OH), 7.50 to 7.45 (m, 9H, H-3, H-7, H-11), 6.85 (d, ³J_{6,7}=8.1, 3H, H-6), 5.34 (s, 6H, H-9), 3.78 (s, 9H, H-8). ¹³C NMR (DMSO-d6): δ 165.3 (C-1), 151.6 (C-5), 147.3 (C-4), 137.1 (C-10), 126.5 (C-11), 123.5 (C-7), 120.1 (C-2), 115.1 (C-6), 112.4 (C-3), 65.2 (C-9), 55.5 (C-8). Mp: 161° C. Anal. Calcd for C₃₃H₃₀O₁₂.3/2CH₂Cl₂: C, 59.89; H, 4.68. Found: C, 59.84; H, 4.72 (equivalent to 8% of CH₂Cl₂ w/w). MS (ESI+) m/z 641.1625 (MNa+), 0.6 ppm. IR-FT: 3378.86; 2941.00; 1701.97; 1597.35; 1528.19; 1516.96.

### [3,5-bis[(4-hydroxy-3-fluoro-benzoyl)oxymethyl]phenyl]methyl 4-hydroxy-3-fluoro-benzoate. DLT95-F

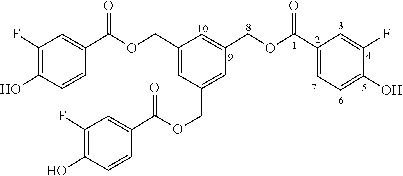

Yield=15%, R_{f}=0.4 (CH₂Cl₂/MeOH: 9/1), RP-HPLC: purity=98.4% (254 nm), t_{R}=3.96 min, ¹H NMR (DMSO-d6): δ 10.91 (bs, 3H, OH), 7.68 to 7.7.65 (m, 6H, H-3, H-7), 7.51 (s, 3H, H-10), 7.03 (t, ³J_{6,7}=⁴J_{6-F}=9.0, 2H, H-6), 5.34 (s, 6H, H-8). ¹³C NMR (DMSO-d6): δ 164.5 (d, ⁴J_{1-F}=2.3, C-1), 150.4 (d, ¹J_{4-F}=240.7, C-4), 149.1 (d, ²J_{5-F}=12.0, C-5), 137.0 (C-9), 126.8 (C-10), 126.7 (d, ⁴J_{7-F}=2.3, C-7), 120.5 (d, ³J_{2-F}=6.0, C-2), 117.6 (d, ³J_{6-F}=3.0, C-6), 117.0 (d, ²J_{3-F}=19.5.0, C-3), 65.6 (C-8). Mp: 88° C. Anal. Calcd for C₃₀H₂₁F₃O₉1/4CH₂Cl₂: C, 60.18; H, 3.59. Found: C, 60.16; H, 4.00. IR-FT: 3382.35; 2957.89; 1702.31; 1617.81; 1597.99; 1518.77.

### [3,5-bis[(4-hydroxy-3-chloro-benzoyl)oxymethyl]phenyl]methyl 4-hydroxy-3-chloro-benzoate. DLT95-Cl

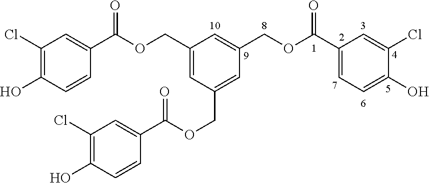

Yield=10%, R_{f}=0.27 (CH₂Cl₂/MeOH: 98/2), RP-HPLC: purity=89.8% (210 nm), t_{R}=2.73 min, ¹H NMR (DMSO-d6): δ 11.3 (bs, 3H, OH), 7.88 (d, ⁴J_{3,7}=1.8, 3H, H-3), 7.79 (dd, ³J_{6,7}=8.7, ⁴J_{3,7}=1.8, 3H, H-7), 7.51 (s, 3H, H-10), 7.05 (d, ³J_{6,7}=8.7, 3H, H-6), 5.34 (s, 6H, H-8). ¹³C NMR (DMSO-d6): δ 164.3 (C-1), 157.7 (C-5), 136.9 (C-4), 131.0 (C-3), 129.8 (C-7), 126.9 (C-2), 121.1 (C-4), 119.9 (C-10), 116.4 (C-6). Mp: 178° C. Anal. Calcd for C₃₀H₂₁Cl₃O₉1/8CH₂Cl₂: C, 56.32; H, 3.33. Found: C, 56.43; H, 3.58. IR-FT: 3393.09; 2961.56; 1690.44; 1601.50; 1579.04; 1500.77.

Exemplary compounds based on Formula (I), (II), (III), (IV), (V) or (VI) are given in Table 1 below.

TABLE 1

| Com- | | X; or Y or | | | | |
|---|---|---|---|---|---|---|
| pound | | Z where | R¹ = | R² = | R³ = | |
| # | Name | indicated | R₄ | R₅ | R₆ | L¹ |
| DLT1 | Ethane-1,2-diyl bis-(4-hydroxy- | O | H | OH | OCH₃ | (CH₂)₂ |
| | 3-methoxybenzoate) | | | | | |
| DLT2 | Propane-1,3-diyl bis-(4- | O | H | OH | OCH₃ | (CH₂)₃ |
| | hydroxy-3-methoxybenzoate) | | | | | |
| DLT3 | Pentane-1,5-diyl bis-(4- | O | H | OH | OCH₃ | (CH₂)₅ |
| | hydroxy-3-methoxybenzoate) | | | | | |
| DLT4 | trans-cyclohexane-1,2-diyl bis- | O | H | OH | OCH₃ | trans-cyclohexane |
| | (4-hydroxy-3- | | | | | |
| | methoxybenzoate) | | | | | |
| DLT5 | cis/trans-cyclohexane-1,3-diyl | O | H | OH | OCH₃ | cis/trans-cyclohexane |
| | bis-(4-hydroxy-3- | | | | | |
| | methoxybenzoate) | | | | | |
| DLT6 | cis/trans-cyclohexane-1,4-diyl | O | H | OH | OCH₃ | cis/trans-cyclohexane |
| | bis-(4-hydroxy-3- | | | | | |
| | methoxybenzoate) | | | | | |
| DLT7 | S,S-trans-cyclohexane-1,2-diyl | O | H | OH | OCH₃ | S,S-trans- |
| | bis-(4-hydroxy-3- | | | | | cyclohexane |
| | methoxybenzoate) | | | | | |
| DLT8 | R,R-trans-cyclohexane-1,2-diyl | O | H | OH | OCH₃ | R,R-trans- |
| | bis-(4-hydroxy-3- | | | | | cyclohexane |
| | methoxybenzoate) | | | | | |
| DLT9 | cis-cyclohexane-1,2-diyl bis-(4- | O | H | OH | OCH₃ | cis-cyclohexane |
| | hydroxy-3-methoxybenzoate) | | | | | |
| DLT10 | Butane-1,4-diyl bis-(4-hydroxy- | O | H | OH | OCH₃ | (CH₂)₄ |
| | 3-methoxybenzoate) | | | | | |
| DLT11 | Hexane-1,6-diyl bis-(4- | O | H | OH | OCH₃ | (CH₂)₆ |
| | hydroxy-3-methoxybenzoate) | | | | | |
| DLT12 | Heptane-1,7-diyl bis-(4- | O | H | OH | OCH₃ | (CH₂)₇ |
| | hydroxy-3-methoxybenzoate) | | | | | |
| DLT13 | Octane-1,8-diyl bis-(4-hydroxy- | O | H | OH | OCH₃ | (CH₂)₈ |
| | 3-methoxybenzoate) | | | | | |
| DLT14 | cis-cyclopentane-1,2-diyl bis- (4-hydroxy-3- methoxybenzoate) | O | H | OH | OCH₃ | 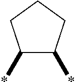 |
| DLT15 | S,S-trans-cyclopentane-1,2- diyl bis-(4-hydroxy-3- methoxybenzoate) | O | H | OH | OCH₃ | 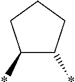 |
| DLT16 | R,R-trans-cyclopentane-1,2- diyl bis-(4-hydroxy-3- methoxybenzoate) | O | H | OH | OCH₃ | 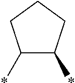 |
| DLT17 | cis-cyclopentane-1,3-diyl bis- (4-hydroxy-3- methoxybenzoate) | O | H | OH | OCH₃ | 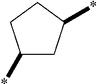 |
| DLT18 | S,S-trans-cyclopentane-1,3- diyl bis-(4-hydroxy-3- methoxybenzoate) | O | H | OH | OCH₃ | 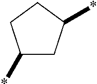 |
| DLT19 | R,R-trans-cyclopentane-1,3- diyl bis-(4-hydroxy-3- methoxybenzoate) | O | H | OH | OCH₃ | 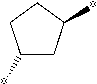 |
| DLT20 | 2-hydroxypropane-1,3-diyl bis(4-hydroxy-3- methoxybenzoate) | O | H | OH | OCH₃ | 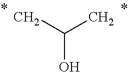 |
| DLT21 | S,S-trans-cycloheptane-1,2- diyl bis-(4-hydroxy-3- methoxybenzoate) | O | H | OH | OCH₃ | 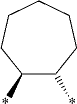 |
| DLT22 | S,S-trans-cycloheptane-1,2- diyl bis-(4-hydroxy-3- methoxybenzoate) | O | H | OH | OCH₃ | 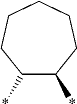 |
| DLT23 | Ethane-1,2-diyl bis-(4-hydroxy- | O | H | OH | Cl | (CH₂)₂ |
| | 3-chlorobenzoate) | | | | | |
| DLT24 | [2-[(4-hydroxy-3-methoxy- benzoyl)oxymethyl]phenyl] methyl 4-hydroxy-3-methoxy- benzoate; methanol | O | H | OCH₃ | OH | 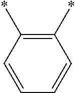 |
| DLT25 | [3-[(4-hydroxy-3-methoxy- benzoyl)oxymethyl]phenyl] methyl 4-hydroxy-3-methoxy- benzoate | Y = O | H | OCH₃ | OH | 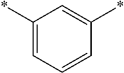 |
| DLT26 | 6-(3,4-dimethoxybenzoyl) | Y = O | H | OCH₃ | OCH₃ | (CH₂)₆ |
| | oxyhexyl 3,4-dimethoxy- | | | | | |
| | benzoate | | | | | |
| DLT27 | [(E)-4-(4-hydroxy-3-methoxy- benzoyl)oxybut-2-enyl] 4- hydroxy-3-methoxy-benzoate; methanol) | Y = O | H | OH | OCH₃ | 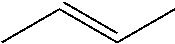 |
| DLT28 | 1,4-oxybut-2-ynyl bis(4- hydroxy-3-methoxy-benzoate) | Y = O | H | OCH₃ | OH | 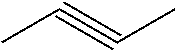 |
| DLT29 | 6-(3-hydroxy-4-methoxy- | Y = O | H | OH | OCH₃ | (CH₂)₆ |
| | benzoyl)oxyhexyl 3-hydroxy-4- | | | | | |
| | methoxy-benzoate | | | | | |
| DLT30 | R,R-trans-cyclohexane-1,2- | O | H | OH | Cl | R,R-trans- |
| | diyl bis-(4-hydroxy-3- | | | | | cyclohexane |
| | chlorobenzoate) | | | | | |
| DLT31 | Ethane-1,2-diyl bis-(3,4,5- | O | OH | OH | OH | (CH₂)₂ |
| | trihydroxybenzoate) | | | | | |
| DLT32 | cis-cyclohexane-1,2-diyl bis- | O | OH | OH | OH | cis-cyclohexane |
| | (3,4,5-trihydroxybenzoate) | | | | | |
| DLT33 | S,S-trans-cyclohexane 1,2-diyl | O | OH | OH | OH | S,S-trans- |
| | bis-(3,4,5-trihydroxybenzoate) | | | | | cyclohexane |
| DLT34 | R,R-trans-cyclohexane 1,2-diyl | O | OH | OH | OH | R,R-trans- |
| | bis-(3,4,5-trihydroxybenzoate) | | | | | cyclohexane |
| DLT35 | N,N′-Ethane-1,2-diyl bis-(4- | NH | H | OH | OCH₃ | (CH₂)₂ |
| | hydroxy-3-methoxybenzamide) | | | | | |
| DLT36 | N,N′-Propane-1,3-diyl bis-(4- | NH | H | OH | OCH₃ | (CH₂)₃ |
| | hydroxy-3-methoxybenzamide) | | | | | |
| DLT37 | N,N′-Pentane-1,5-diyl bis-(4- | NH | H | OH | OCH₃ | (CH₂)₅ |
| | hydroxy-3-methoxybenzamide) | | | | | |
| DLT38 | N,N′-trans-cyclohexane-1,2- | NH | H | OH | OCH₃ | trans-cyclohexane |
| | diyl bis-(4-hydroxy-3- | | | | | |
| | methoxybenzamide) | | | | | |
| DLT39 | N,N′-cis/trans-cyclohexane- | NH | H | OH | OCH₃ | cis/trans-cyclohexane |
| | 1,3-diyl bis-(4-hydroxy-3- | | | | | |
| | methoxybenzamide) | | | | | |
| DLT40 | N,N′-cis/trans-cyclohexane- | NH | H | OH | OCH₃ | cis/trans-cyclohexane |
| | 1,4-diyl bis-(4-hydroxy-3- | | | | | |
| | methoxybenzamide) | | | | | |
| DLT41 | N,N′-cis-cyclohexane-1,2-diyl | NH | H | OH | OCH₃ | cis-cyclohexane |
| | bis-(4-hydroxy-3- | | | | | |
| | methoxybenzamide) | | | | | |
| DLT42 | N,N′-S,S-trans-cyclohexane- | NH | H | OH | OCH₃ | S,S-trans- |
| | 1,2-diyl bis-(4-hydroxy-3- | | | | | cyclohexane |
| | methoxybenzamide) | | | | | |
| DLT43 | N,N′-R,R-trans-cyclohexane- | NH | H | OH | OCH₃ | R,R-trans- |
| | 1,2-diyl bis-(4-hydroxy-3- | | | | | cyclohexane |
| | methoxybenzamide) | | | | | |
| DLT44 | N,N′-Butane-1,4-diyl bis-(4- | NH | H | OH | OCH₃ | (CH₂)₄ |
| | hydroxy-3-methoxybenzamide) | | | | | |
| DLT45 | N,N′-Hexane-1,6-diyl bis-(4- | NH | H | OH | OCH₃ | (CH₂)₆ |
| | hydroxy-3-methoxybenzamide) | | | | | |
| DLT46 | N,N′-Heptane-1,7-diyl bis-(4- | NH | H | OH | OCH₃ | (CH₂)₇ |
| | hydroxy-3-methoxybenzamide) | | | | | |
| DLT47 | N,N′-Octane-1,8-diyl bis-(4- | NH | H | OH | OCH₃ | (CH₂)₈ |
| | hydroxy-3-methoxybenzamide) | | | | | |
| DLT48 | N,N′-cis-cyclopentane-1,2-diyl bis-(4-hydroxy-3- methoxybenzamide) | NH | H | OH | OCH₃ | 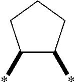 |
| DLT49 | N,N′-S,S-trans-cyclopentane- 1,2-diyl bis-(4-hydroxy-3- methoxybenzamide) | NH | H | OH | OCH₃ | 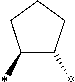 |
| DLT50 | N,N′-R,R-trans-cyclopentane- 1,2-diyl bis-(4-hydroxy-3- methoxybenzamide) | NH | H | OH | OCH₃ | 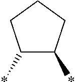 |
| DLT51 | N,N′-cis-cyclopentane-1,3-diyl bis-(4-hydroxy-3- methoxybenzamide) | NH | H | OH | OCH₃ | 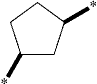 |
| DLT52 | N,N′-S,S-trans-cyclopentane- 1,3-diyl bis-(4-hydroxy-3- methoxybenzamide) | NH | H | OH | OCH₃ | 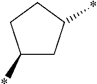 |
| DLT53 | N,N′-R,R-trans-cyclopentane- 1,3-diyl bis-(4-hydroxy-3- methoxybenzamide) | NH | H | OH | OCH₃ | 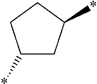 |
| DLT54 | N,N′-(2-aminopropane)-1,3-diyl | NH | H | OH | OCH₃ | CH₂CH(NH₂)CH₂ |
| | bis(4-hydroxy-3- | | | | | |
| | methoxybenzamide) | | | | | |
| DLT55 | N,N′-S,S-trans-cycloheptane- 1,2-diyl bis-(4-hydroxy-3- methoxybenzamide) | NH | H | OH | OCH₃ | 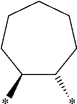 |
| DLT56 | N,N′-S,S-trans-cycloheptane- 1,2-diyl bis-(4-hydroxy-3- methoxybenzamide) | NH | H | OH | OCH₃ | 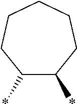 |
| DLT57 | N,N′-Ethane-1,2-diyl bis-(4- | NH | H | OH | Cl | (CH₂)₂ |
| | hydroxy-3-chlorobenzamide) | | | | | |
| DLT58 | N,N′-Ethane-1,2-diyl bis-(4- | NH | OCH₃ | OH | OCH₃ | (CH₂)₂ |
| | hydroxy-3,5- | | | | | |
| | dimethoxybenzamide) | | | | | |
| DLT59 | N,N′-S,S-trans-cyclohexane- | NH | OCH₃ | OH | OCH₃ | S,S-trans- |
| | 1,2-diyl bis-(4-hydroxy-3,5- | | | | | cyclohexane |
| | dimethoxybenzamide) | | | | | |
| DLT60 | N,N′-S,S-trans-cyclohexane- | NH | H | OH | Cl | S,S-trans- |
| | 1,2-diyl bis-(4-hydroxy-3- | | | | | cyclohexane |
| | chlorobenzamide) | | | | | |
| DLT61 | N,N′-cis-cyclohexane-1,2-diyl | NH | OCH₃ | OH | OCH₃ | cis-cyclohexane |
| | bis-(4-hydroxy-3,5- | | | | | |
| | dimethoxybenzamide) | | | | | |
| DLT62 | N,N′-cis-cyclohexane-1,2-diyl | NH | H | OH | Cl | cis-cyclohexane |
| | bis-(4-hydroxy-3- | | | | | |
| | chlorobenzamide) | | | | | |
| DLT63 | N,N′-R,R-trans-cyclohexane- | NH | OCH₃ | OH | OCH₃ | R,R-trans- |
| | 1,2-diyl bis-(4-hydroxy-3,5- | | | | | cyclohexane |
| | dimethoxybenzamide) | | | | | |
| DLT64 | N,N′-R,R-trans-cyclohexane- | NH | H | OH | Cl | R,R-trans- |
| | 1,2-diyl bis-(4-hydroxy-3- | | | | | cyclohexane |
| | chlorobenzamide) | | | | | |
| DLT65 | N,N′-Ethane-1,2-diyl bis-(3,4,5- | NH | OH | OH | OH | (CH₂)₂ |
| | trihydroxybenzamide) | | | | | |
| DLT66 | N,N′-cis-cyclohexane-1,2-diyl | NH | OH | OH | OH | cis-cyclohexane |
| | bis-(3,4,5- | | | | | |
| | trihydroxybenzamide) | | | | | |
| DLT67 | N,N′-S,S-trans-cyclohexane | NH | OH | OH | OH | S,S-trans- |
| | 1,2-diym bis- | | | | | cyclohexane |
| | (3,4,68trihydroxybenzamide) | | | | | |
| DLT68 | N,N′-R,R-trans-cyclohexane | NH | OH | OH | OH | R,R-trans- |
| | 1,2-diyl bis-(3,4,5- | | | | | cyclohexane |
| | trihydroxybenzamide) | | | | | |
| DLT69^{$} | 3-(hydroxymethyl)-3- | O | H | OH | OCH₃ | 4-(hydroxymethyl)-4- |
| | hydroxycyclohexyl-1,5,6-diyl | | | | | hydroxy-cyclohexyl- |
| | tris(4-hydroxy-3- | | | | | (4-hydroxy-3- |
| | methoxybenzoate) | | | | | methoxybenzoate) |
| | 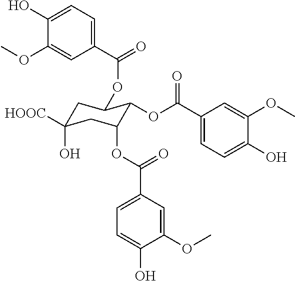 | | | | | 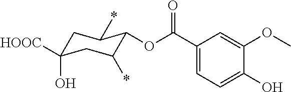 |
| DLT70 | 2-hydroxypropane-1,3-diyl bis(3-chloro-4- hydroxybenzoate) | O | H | OH | Cl | 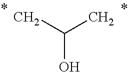 |
| DLT71 | 2-hydroxypropane-1,3-diyl bis (4-hydroxy-3,5- dimethoxybenzoate) | O | OMe | OH | OMe | 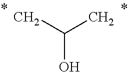 |
| DLT73 | Propane-1,3-diyl bis-(4- | O | H | OH | Cl | (CH₂)₃ |
| | hydroxy-3-chlorobenzoate) | | | | | |
| DLT74 | Pentane-1,5-diyl bis-(4- | O | H | OH | Cl | (CH₂)₅ |
| | hydroxy-3-chlorobenzoate) | | | | | |
| DLT75 | trans-cyclohexane-1,2-diyl bis- | O | H | OH | Cl | trans-cyclohexane |
| | (4-hydroxy-3-chlorobenzoate) | | | | | |
| DLT76 | cis/trans-cyclohexane-1,3-diyl | O | H | OH | Cl | cis/trans-cyclohexane |
| | bis-(4-hydroxy-3- | | | | | |
| | chlorobenzoate) | | | | | |
| DLT78 | cis/trans-cyclohexane-1,4-diyl | O | H | OH | Cl | cis/trans-cyclohexane |
| | bis-(4-hydroxy-3- | | | | | |
| | chlorobenzoate) | | | | | |
| DLT79 | Butane-1,4-diyl bis-(4-hydroxy- | O | H | OH | Cl | (CH₂)₄ |
| | 3-chlorobenzoate) | | | | | |
| DLT80 | Hexane-1,6-diyl bis-(4- | O | H | OH | Cl | (CH₂)₆ |
| | hydroxy-3-chlorobenzoate) | | | | | |
| DLT81 | Propane-1,3-diyl bis-(4- | O | OMe | OH | OMe | (CH₂)₃ |
| | hydroxy-3-chlorobenzoate) | | | | | |
| DLT82 | Pentane-1,5-diyl bis-(4- | O | OMe | OH | OMe | (CH₂)₅ |
| | hydroxy-3,5- | | | | | |
| | dimethoxybenzoate) | | | | | |
| DLT83 | trans-cyclohexane-1,2-diyl bis- | O | OMe | OH | OMe | trans-cyclohexane |
| | (4-hydroxy-3,5- | | | | | |
| | dimethoxybenzoate) | | | | | |
| DLT84 | cis/trans-cyclohexane-1,3-diyl | O | OMe | OH | OMe | cis/trans-cyclohexane |
| | bis-(4-hydroxy-3,5- | | | | | |
| | dimethoxybenzoate) | | | | | |
| DLT85 | cis/trans-cyclohexane-1,4-diyl | O | OMe | OH | OMe | cis/trans-cyclohexane |
| | bis-(4-hydroxy-3,5- | | | | | |
| | dimethoxybenzoate) | | | | | |
| DLT86 | Butane-1,4-diyl bis-(4-hydroxy- | O | OMe | OH | OMe | (CH₂)₄ |
| | 3,5-dimethoxybenzoate) | | | | | |
| DLT87 | Hexane-1,6-diyl bis-(4- | O | OMe | OH | OMe | (CH₂)₆ |
| | hydroxy-3,5- | | | | | |
| | dimethoxybenzoate) | | | | | |
| DLT88 | S,S-trans-cyclohexane-1,2-diyl | O | H | OH | Cl | S,S-trans- |
| | bis-(4-hydroxy-3- | | | | | cyclohexane |
| | chlorobenzoate) | | | | | |
| DLT89 | cis-cyclohexane-1,2-diyl bis-(4- | O | OCH₃ | OH | OCH₃ | cis-cyclohexane |
| | hydroxy-3,5- | | | | | |
| | dimethoxybenzoate | | | | | |
| DLT90 | cis-cyclohexane-1,2-diyl bis-(4- | O | H | OH | Cl | cis-cyclohexane |
| | hydroxy-3-chlorobenzoate) | | | | | |
| DLT91 | R,R-trans-cyclohexane-1,2-diyl | O | OCH₃ | OH | OCH₃ | R,R-trans- |
| | bis-(4-hydroxy-3,5- | | | | | cyclohexane |
| | dimethoxybenzoate | | | | | |
| DLT92 | S,S-trans-cyclohexane-1,2-diyl | O | OCH₃ | OH | OCH₃ | S,S-trans- |
| | bis-(4-hydroxy-3,5- | | | | | cyclohexane |
| | dimethoxybenzoate | | | | | |
| DLT94 | Ethane-1,2-diyl bis-(4-hydroxy- | O | OCH₃ | OH | OCH₃ | (CH₂)₂ |
| | 3,5-dimethoxybenzoate) | | | | | |
| DLT95 | [3,5-bis-[(4-hydroxy-3- methoxy-benzoyl)-oxymethyl]- phenyl]-methyl-4-hydroxy-3- methoxy-benzoate | Z = OCH₂ | OCH₃ | OH | H | 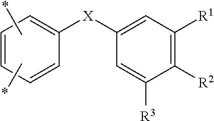 |
| DLT95- F | [3,5-bis[(4-hydroxy-3-fluoro- benzoyl)oxymethyl]-phenyl]- methyl-4-hydroxy-3-fluoro- benzoate | Z = OCH₂ | F | OH | H | 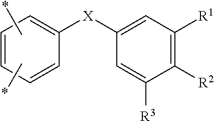 |
| DLT95- Cl | [3,5-bis-[(4-hydroxy-3-chloro- benzoyl)-oxymethyl]-phenyl]- methyl-4-hydroxy-3-chloro- benzoate | Z = OCH₂ | Cl | OH | H | 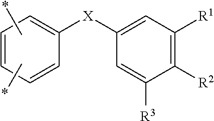 |

In each of the above structures, the L¹ group can optionally have one or more substituents each independently selected from the group comprising C₁₋₆alkyl, CO₂H, vanillic acid, and C₁₋₆alkyloxycarbonyl.
* The asterisk is used herein to indicate the point at which a mono- or bivalent radical depicted is connected to the structure to which it relates and of which the radical forms part.
^{$}For this molecule (DLT69), the three-steps synthetic path envisaged is depicted in scheme 3 below.
When “Y =” or “Z =” is indicated in the column “X”, the compound is based respectively on general formula's (I) or (VI).

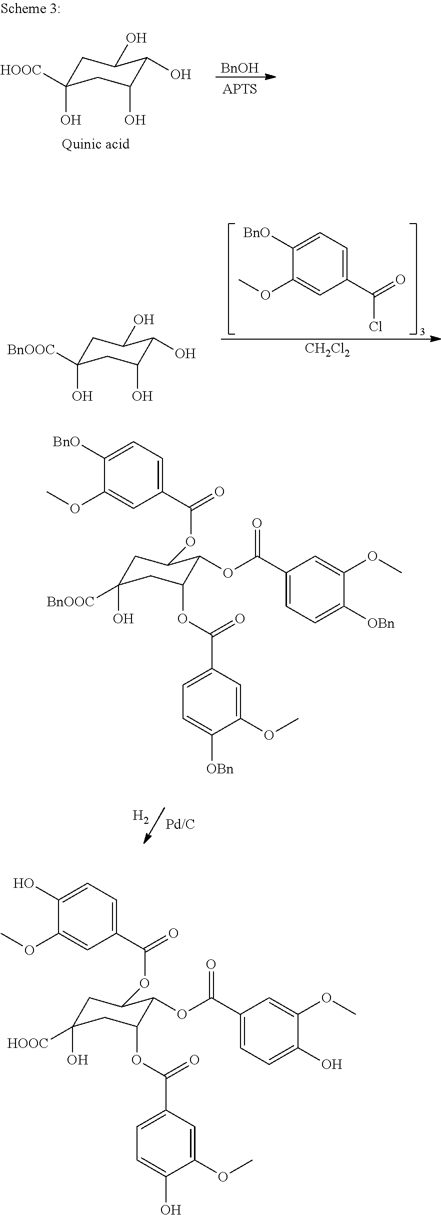

All synthesized compounds can be analyzed for their capability to modify intra-cellular ion concentrations, with focus on Cl⁻, Na⁺, K⁺ and Ca²⁺⁺.

### Example 2

### In Vitro Characterization of the Biological Effects of the Compounds According to the Invention

### A/ Effect on Overall Cell Growth

MTT tests were performed in order to rapidly, i.e. within 5 days, measure the effect of compounds of this invention on the overall cell growth. The test measured the number of metabolically active living cells that were able to transform the yellow product 3-(4,5-dimethylthiazol-2-yl)-2,5-diphenyl tetrazolium bromide (herein referred as MTT) into the blue product formazan dye by mitochondrial reduction. The amount of formazan obtained at the end of the experiment, measured by means of a spectrophotometer, is directly proportional to the number of living cells. Optical density determination thus enabled a quantitative measurement of the effect of the investigated compounds as compared to the control condition (untreated cells) and/or to other reference compounds.

Eleven human cancer cell lines and one mouse melanoma cell-line (B16F10) described in Table 2 were used in the following MTT tests. These cancer cell lines cover seven histological cancer types including prostate (PC3), glioma (Hs683, T98G, U373, melanoma (VM21, VM28), non-small-cell-lung (A549), breast (MCF-7), colon (LoVo) and oesophageal (OE21, OE33) cancers.

To perform the assay, cells were allowed to grow in 96-well micro-wells with a flat bottom with an amount of 100 μl of cell suspension per well with 5,000 to 8,000 cells/well depending on the cell type used. Each cell line was seeded in its appropriate culture medium.

The detailed experimental procedure was the following: after a 24-hour period of incubation at 37° C., the culture medium was replaced by 100 μl of fresh medium in which the tested compound was previously dissolved, at the following molar concentrations: 10⁻⁸ M, 5.10⁻⁸ M, 10⁻⁷ M, 5.10⁻⁷ M, 10⁻⁶ M, 5.10⁻⁶ M, 10⁻⁵ M, 5.10⁻⁵ M and 10⁻⁴ M. Each experiment was performed in sestuplicates (6 times).

After 72 hours of incubation at 37° C. with (experimental conditions) or without (control condition) the compound to be tested, the medium was replaced by 100 μl MTT dissolved in RPMI (1640 without phenol red) at a concentration of 0.5 or 1 mg/ml. The micro-wells were subsequently incubated during 3 hours and a half at 37° C. and centrifuged at 1300 rpm during 10 minutes. MTT was removed and formazan crystals formed were dissolved in 100 μl DMSO. The micro-wells were shaken for 5 minutes and read on a spectrophotometer at wavelengths of 570 nm (maximal formazan absorbance).

For each experimental condition, the mean optical density was calculated, allowing the determination of the percentage of remaining living cells in comparison to the control.

Table 3 shows the IC₅₀ (representing the range of concentration of the compound tested that resulted in a 50% inhibition of overall tumour cells growth) for each compound in each cell line investigated.

The origin of all the cell lines we used, along with their biological characteristics, and the validation procedures of the MTT colorimetric assay as employed here are fully detailed in Van Quaquebeke et al., 2005, J Med Chem 48:849-856; Ingrassia et al., 2009, J Med Chem 52:1100-1114; Mathieu et al., 2009, J Cell Mol Med, Feb. 20, 2009.

TABLE 2
IC₅₀ (μM) values IC50: Concentration of drug needed in order to inhibit cell population growth by 50%

| | In Vitro IC₅₀ Growth Inhibitory Values (μM; * = >100 μM; **: >1000 μM)^{a} | | | | | | | | | | | | |
|---|---|---|---|---|---|---|---|---|---|---|---|---|---|
| | U373 | Hs683 | A549 | PC-3 | MCF-7 | LoVo | T98G | OE21 | OE33 | VM21 | VM48 | m ± SEM | B16F10 |
| Control: | ** | ** | ** | ** | ** | ** | ** | ** | ** | ** | ** | ** | ** |
| vanillic | | | | | | | | | | | | | |
| acid | | | | | | | | | | | | | |
| DLT1 | 41 | 82 | 69 | 57 | 45 | 10 | 59 | 56 | 63 | 55 | 53 | 53 + 5 | 28 |
| DLT2 | * | * | * | 88 | 89 | 50 | * | * | 64 | 72 | 68 | * | 45 |
| DLT10 | 98 | * | 65 | 67 | 80 | 54 | 91 | * | 67 | 60 | 52 | * | 94 |
| DLT3 | 69 | * | * | * | * | 94 | * | * | * | * | * | * | * |
| DLT11 | 22 | 63 | 27 | 39 | 39 | 34 | 56 | 68 | 63 | 71 | 56 | 49 + 5 | 22 |
| DLT12 | 44 | 50 | 51 | 44 | 56 | 44 | 58 | 46 | 47 | 32 | 38 | 46 + 2 | 10 |
| DLT13 | 38 | 40 | 39 | 30 | 35 | 94 | 45 | 62 | 28 | 28 | 26 | 42 + 6 | 4 |
| DLT14 | 36 | 27 | 24 | 36 | 27 | 24 | 32 | 28 | 28 | 33 | 22 | 29 + 1 | 9 |
| DLT15 | 30 | 25 | 30 | 26 | 23 | 28 | 30 | 28 | 28 | 35 | 26 | 28 + 1 | 6 |
| DLT16 | 31 | 22 | 21 | 25 | 25 | 29 | 32 | 27 | 28 | 26 | 29 | 27 + 1 | 4 |
| DLT29 | 68 | 50 | 54 | 90 | 55 | 30 | * | 44 | 52 | 74 | 82 | * | 68 |
| DLT19 | 35 | 30 | 32 | 42 | 25 | 18 | 31 | 34 | 36 | 37 | 53 | 34 + 3 | 26 |
| DLT26 | 46 | 49 | 46 | 86 | * | 82 | * | 49 | * | * | * | * | * |
| DLT18 | * | 46 | 43 | 43 | 44 | 41 | * | 53 | 64 | * | 59 | * | 30 |
| DLT22 | 54 | 44 | 46 | 88 | 69 | 44 | 24 | 49 | 55 | 51 | 50 | 52 + 5 | 10 |
| DLT30 | 31 | 10 | 18 | 44 | 21 | 26 | 11 | 41 | 34 | 53 | 58 | 32 + 5 | 1 |
| DLT34 | 37 | 24 | 32 | 68 | 28 | 34 | 31 | 37 | 45 | 91 | 28 | 41 + 6 | 0.3 |
| DLT23 | 52 | 53 | 72 | 81 | 53 | 48 | 45 | 31 | 73 | 34 | 84 | 57 + 5 | 27 |
| DLT33 | 35 | 32 | 30 | 44 | 58 | 43 | 38 | 43 | 66 | 38 | 51 | 43 + 3 | 22 |
| DLT27 | * | * | * | * | * | * | * | 73 | * | * | * | * | 57 |
| DLT28 | 39 | 40 | 40 | 46 | 39 | 36 | 72 | 76 | 61 | 42 | 60 | 50 ± 4 | 36 |
| DLT24 | 65 | 47 | 48 | 42 | 44 | 46 | 64 | 56 | 66 | 48 | 58 | 53 ± 2 | 24 |
| DLT25 | 66 | 58 | 63 | 42 | 57 | 26 | 46 | 34 | 45 | 81 | 51 | 52 ± 5 | 37 |
| DLT7 | 20 | 57 | 43 | 59 | 47 | 44 | 70 | 41 | 66 | 35 | 36 | 47 ± 4 | 49 |
| DLT8 | 53 | * | 76 | 77 | 75 | 64 | * | 45 | 77 | 77 | 72 | * | 59 |
| DLT9 | 43 | 49 | 38 | 35 | 46 | 28 | 76 | 55 | 56 | 44 | 57 | 48 ± 3 | 38 |
| DLT5 | 27 | 69 | 65 | 34 | 32 | 31 | 72 | 46 | 55 | 47 | 47 | 48 ± 5 | 40 |
| DLT40 | * | 95 | * | 87 | 80 | 78 | * | 81 | * | * | 98 | * | 36 |
| DLT31 | 28 | 27 | 23 | 30 | 55 | 29 | 28 | 16 | 19 | 37 | 35 | 30 ± 3 | 41 |
| DLT31- | 37 | 25 | 26 | 44 | 41 | 48 | 37 | 73 | 71 | 65 | 61 | 33 ± 2 | 31 |
| F | | | | | | | | | | | | | |
| DLT31- | 31 | 31 | 26 | 36 | 33 | 33 | 25 | 28 | 40 | 35 | 32 | 29 ± 1 | 7 |
| Cl | | | | | | | | | | | | | |
| DLT36 | 38 | 31 | 26 | 40 | 25 | 32 | 40 | 28 | 36 | 49 | 43 | 35 ± 2 | 38 |
| DLT37 | 91 | 80 | 52 | * | 71 | 41 | 55 | 81 | * | * | * | * | * |

The purity of the DLT95 compounds was: 99% for DLT95, 98% for DLT95F and 92% for DLT95Cl.

wherein compounds 1-7 in Table 2 have the following general formula:
with linear carbon chains.

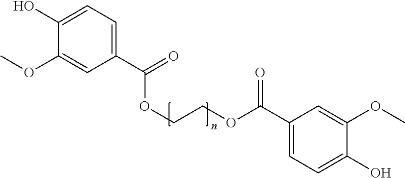

and wherein compounds 8-12 in Table 2 have the following general formula:

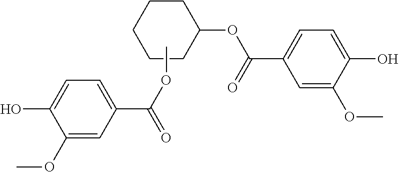

wherein 8-11 are of the cyclo-1,2 type and 12 is of the cyclo-1,3 type.

Wherein compounds 13-15 in Table 2 have the following general formula:

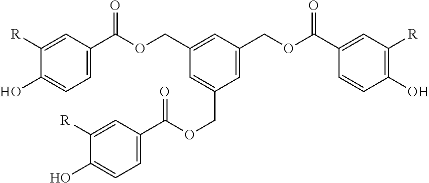

wherein for compound
13: R═Ome: 11%
14: R═F: 15%
15: R═Cl: 10%

### B/ Impairment of Cell Proliferation and Cell Migration Triggered by the Compound of the Present Invention in Cancer Cells

As the MTT test is based on the mitochondria functions, we also investigated the effects of the di- and tri-vanilloyl derivatives according to the invention on cell proliferation, migration and morphology by means of a cellular imaging approach (Debeir et al., Cytometry 60:29-40, 2004; Debeir et al., IEEE Trans Med Imaging 24:697-711, 2005) either in the human U373-MG glioma cell line, which is apoptosis-resistant but autophagy-sensitive (Lefranc F et al., Neurosurgery 2008) and in the human A549 non-small-cell-lung cancer cell line, which is apoptosis-resistant and autophagy-resistant (Mijatovic T et al., Neoplasia 2006, May; 8(5):402-12).

Investigations have also been performed in human normal fibroblasts (WS1 and WI38—see Tables 4 and 5). Cellular imaging relied on the use of computer-assisted phase-contrast microscopy making possible to film the behaviour of living cells in culture dishes for several days.

Cells were seeded in a 25-cm² flask at a low density, treated or not with the vanilloyl-esters invention (at a concentration of 50 μM) and filmed thereafter for a period of 72 h. The experiment was conducted in quadruplicates (in duplicates for DLT-95 compounds).

The behaviour of the cells, in terms of morphology, growth and death were thus investigated. The effect on the overall growth was measured by counting the number of cells on the first (0 h) and the last image (72 h) of each film. The global growth ratio (GGR) was then deduced by dividing the number of cells on the last image by the number of cells on the first image. The ratio GGR_{treated cells}/GGR_{control cells}, was further calculated thereby obtaining a value that describes the effect of compounds of the present invention on the overall cell growth. The methodology is fully described and validated in Debeir et al., 2008, Exp Cell Res 314:2985-2998 and Mathieu et al., 2009, J Cell Mol Med, Feb. 20, 2009.

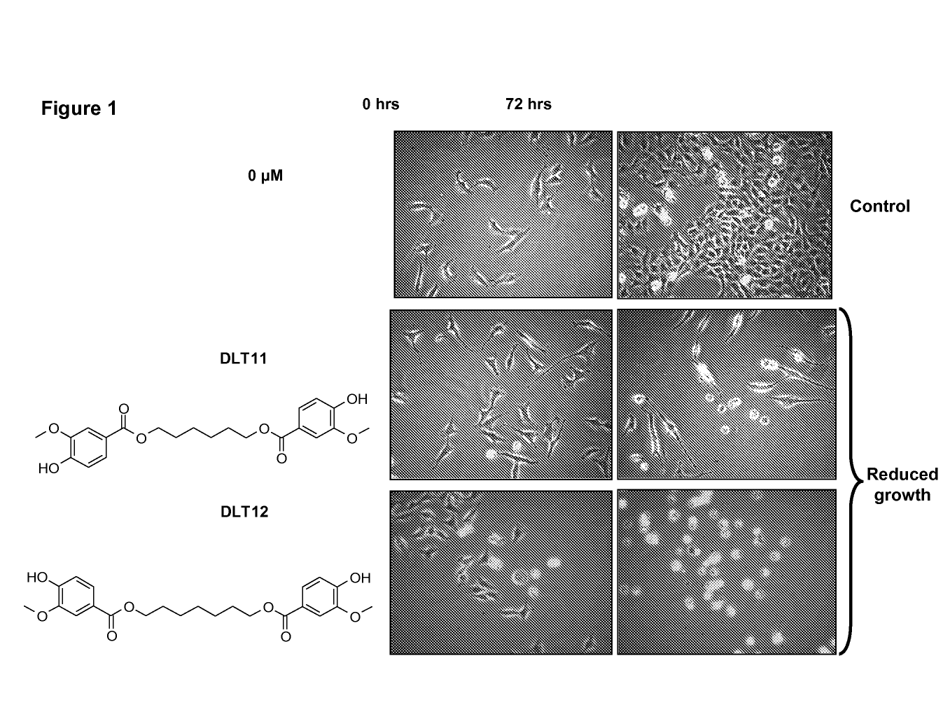

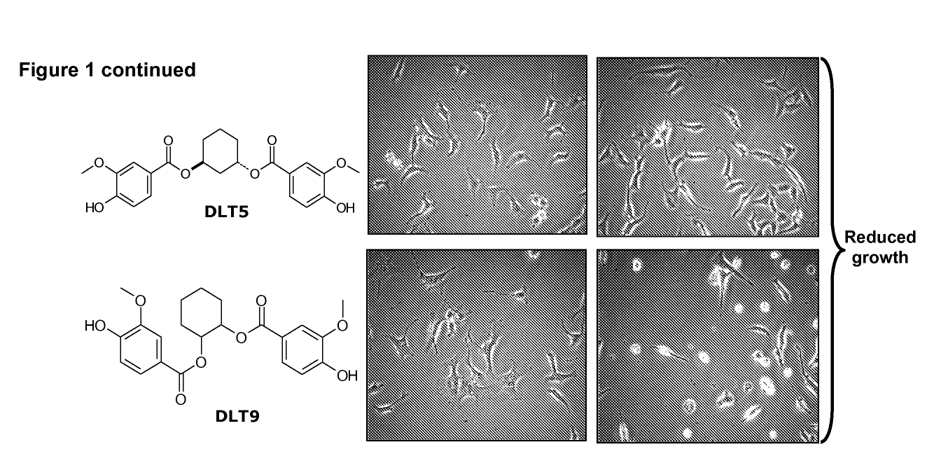

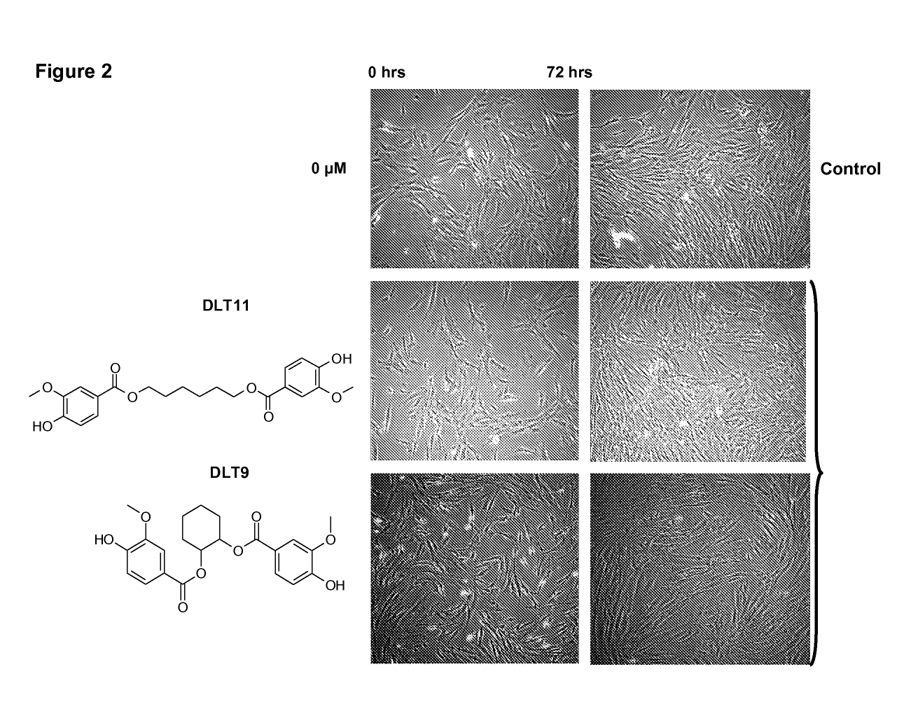

The recordings (not shown) and data obtained clearly show that the compounds according to the invention impair cell morphology and proliferation of human cancer cells. Illustrative pictures (time=0 h and 72 h) of glioblastoma and normal fibroblast cell line left untreated or treated with poly-vanillic compounds according to the invention (50 μM) are provided in FIG. 1 (U373 glioblastoma) and FIG. 2 (normal fibroblasts). The GGR parameter analyses confirmed the marked impairment of cell growth in both cancer cell lines of the poly-vanillic compounds according to the invention (Table 5).

Table 5 summarizes all the data obtained with this assay; the DLT1 and DLT4 compounds according to the invention clearly impair cell morphology, and growth in cancer cell-lines, but not in human fibroblast (non-cancer) cell-lines.

TABLE 4
Human normal cell lines

| Normal | ATCC | | |
|---|---|---|---|
| cell lines | code | Tissue | Literature reference |
| WS1 | | Fibroblasts | In Vitro Cell Dev Biol |
| | | | Anim. 34(8):631-5, 1998 |
| WI38 | | Fibroblasts | Exp Cell Res. 90(1):8-14, 1975 |

TABLE 5
Cellular imaging: recapitulative data

| | | | | Cancer cell | | Normal cell | |
|---|---|---|---|---|---|---|---|
| Global Grow Ratio Treated/Ct | | | | lines | | lines | |
| determined by Video Quantitative | | GGR T/Ct | | (Human) | | (Human) | |
| microscopy | | (50 μM) | | U373 | A549 | WI38 | WS1 |
| 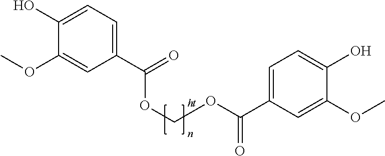 | n = 2 n = 3 n = 4 n = 5 n = 6 n = 7 n = 8 | | DLT-1 DLT-2 DLT-10 DLT-3 DLT-11 DLT-12 DLT-13 | 0.3 0.4 0.5 0.7 0.2 0.2 0.2 | 0.5 1.1 0.2 0.9 0.2 0.2 0.2 | 0.8 | |
| 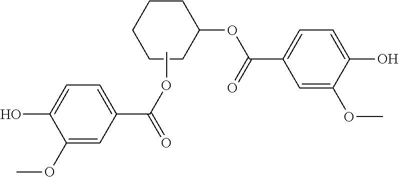 | cyclo-1,2 cyclo-1,3 | mix of trans trans SS trans RR cis mix of trans | DLT-4 DLT-7 DLT-8 DLT-9 DLT-5 | 0.2 0.2 0.5 0.2 0.2 | 0.2 0.2 0.5 0.2 0.1 | 0.9 | 1.0 |
| 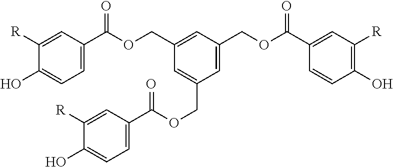 | K = O—CH2 K = F F = Cl | | DLT95 DLT95-F* DLT-95- Cl** | 0.1 0.4 0.5 | | | |

*= 37 μM/
**= 31 μM

### Example 3

### Kinase Inhibition Profile of DLT11 at 20 μM

### Materials and Methods

The kinase inhibition profile of DLT11 at 20 μM was determined using 250 protein kinases. Residual activity values were measured by testing each compound at one concentration in duplicate in each kinase assay. A radiometric protein kinase assay (33 PanQinase®Activity Assay) was used for measuring the kinase activity of 250 protein kinases. All kinase assays were performed in 96-well flashPlates™ from Perkin Elmer (Boston, Mass., USA) in a 50 μl reaction volume. The reaction cocktail was pipetted in 4 steps in the following order:
10 μl of non-radioactive ATP solution (in H₂O)
25 μl of assay buffer/[γ-33P]-ATP mixture
5 μl of test sample in 10% DMSO
10 μl of enzyme/substrate mixture

The assay for all enzymes contained 60 mM HEPES-NaOH, pH 7.5, 3 mM MgCl2, 3 mM MnCl2, 3 μM Na-orthovanadate, 1.2 mM DTT, 50 μg/ml PEG20000, 1 μM ATP/[γ-33P]-ATP (approx. 6×1005 cpm per well), protein kinase, and substrate. All PKC assays (except the PKC-mu and the PKC-nu assay) additionally contained 1 mM CaCl2, 4 mM EDTA, 5 μg/ml Phosphatidylserine and 1 μg/ml 1,2-Dioleyl-glycerol. The MYLK2, CAMK1D, CAMK2A, CAMK2B, CAMK2D, CAMK4, CAMKK2, DAPK2 and EEF2K assays additionally contained 1 μg/ml Calmodulin and 0.5 mM CaCl2. The PRKG1 and PRKG2 assays additionally contained 1 μM cGMP.

### Recombinant Protein Kinases:

The protein kinases were expressed either in Sf9 insect cells or in E. coli as recombinant GST-fusion proteins or as His-tagged proteins. All kinases were produced from human cDNAs. Kinases were purified by affinity chromatography using either GSH-agarose (Sigma) or Ni-NTH-agarose (Qiagen). The purity of the protein kinases was examined by SDS-PAGE/coomassie staining. The identity of the protein kinases was checked by mass spectroscopy.

The reaction cocktails were incubated at 30° C. for 60 minutes. The reaction was stopped with 50 μl of 2% (v/v) H₃PO₄, plates were aspirated and washed two times with 200 μl 0.9% (w/v) NaCl. All assays were performed with a BeckmanCoulter Biomek 2000/SL robotic system.

Incorporation of ³³Pi (counting of “cpm”) was determined with a microplate scintillation counter (Microbeta, Wallac).

For each kinase, the median value of the cpm of three wells with complete reaction cocktails, but without kinase, was defined as “low control” (n=3). This value reflects unspecific binding of radioactivity to the plate in the absence of protein kinase but in the presence of the substrate. Additionally, for each kinase the median value of the cpm of three other wells with the complete reaction cocktail, but without any compound, was taken as the “high control”, i.e. full activity in the absence of any inhibitor (n=3). The difference between high and low control of was taken as 100% activity for each kinase. As part of the data evaluation the low control value of each kinase was subtracted from the high control value as well as from their corresponding “compound values”. The residual activity (in %) for each compound well was calculated by using the following formula: Res. Activity (%)=100×[(cpm of compound−low control)/(high control−low control)]

### Results

250 kinases where tested with DLT11 at 20 μM to establish the kinase inhibition profile of the compound. FIG. 3 shows that 19 out of 250 kinases had their activity inhibited by DLT11.

### Example 4

### Aurora A, B and C Kinase Inhibition Profile of DLT1, DLT2, DLT 7, DLT8, DLT9, DLT11, DLT12 and Vanillic Acid at 8 Different Concentrations

We established dose-response curves for the inhibition of Aurora A, B and C kinases activity for the DLT compounds and vanillic acid at 0, 1, 5, 10, 25, 50, 75 and 100 μM with the same protocol as describe previously (FIG. 4). As can be seen from the figure, all 8 compounds have a marked effect on the Aurora kinases A, B and C, indicating a common target for the DLT compounds of the invention. Note that at a concentration of 20 μM of the DLT compounds, the activity of the three Aurora kinases is reduced by approximately 40-50%, whereas at concentrations of 50 μM, i.e. the concentration used in the anti-proliferation assays of example 2B, the activity of said kinases is reduced by 60-75%.

### Example 5

### Kinase Inhibition Profile of DLT1 and DLT5 at 10 μM

### Materials and Methods

### Buffers

Buffer A: 10 mM MgCl₂, 1 mM EGTA, 1 mM DTT, 25 mM Tris-HCl pH 7.5, 50 μg heparin/ml.
Buffer C: 60 mM β-glycerophosphate, 15 mM p-nitrophenylphosphate, 25 mM Mops (pH 7.2), 5 mM EGTA, 15 mM MgCl₂, 1 mM DTT, 1 mM sodium vanadate, 1 mM phenyl phosphate.

### Kinase Preparations and Assays

Kinase activities were assayed in Buffer A or C, at 30° C., at a final ATP concentration of 15 μM. Blank values were subtracted and activities expressed in % of the maximal activity, i.e. in the absence of inhibitors. Controls were performed with appropriate dilutions of DMSO.

CDK1/cyclin B (M phase starfish oocytes, native), CDK2/cyclin A, CDK2/cyclin E, CDK5/p25 and CDK7/cyclin H (human, recombinant) were prepared as previously described (Leclerc et al., 2001, J. Biol. Chem. 2001, 276, 251-260; Bach et al., 2005, J Biol Chem 280: 31208-31219). Their kinase activity was assayed in buffer C, with 1 mg histone H1/ml, in the presence of 15 μM [gamma-³³P] ATP (3,000 Ci/mmol; 10 mCi/ml) in a final volume of 30 μl. After 30 min incubation at 30° C., 25 μl aliquots of supernatant were spotted onto 2.5×3 cm pieces of Whatman P81 phosphocellulose paper, and, 20 sec later, the filters were washed five times (for at least 5 min each time) in a solution of 10 ml phosphoric acid/liter of water. The wet filters were counted in the presence of 1 ml ACS (Amersham) scintillation fluid.

CDK9/cyclin T (human, recombinant, expressed in insect cells) was assayed as described for CDK1/cyclin B, but using a pRB fragment (a.a.773-928) (3.5 μg/assay) as a substrate.

GSK-3 (porcine brain, native) was assayed, as described for CDK1 but in Buffer A and using a GSK-3 specific substrate (GS-1: YRRAAVPPSPSLSRHSSPHQSpEDEEE) (Seq. ID NO 1) (pS stands for phosphorylated serine) (Primot et al., 2000, Protein Expr. & Purif. 20 (3), 394-404). GS-1 was synthesized by Millegen (Labege, France).

CK1 (porcine brain, native) was assayed as described for CDK1 but using the CK1-specific peptide substrate RRKHAAIGpSAYSITA (Seq. ID NO 2) (Reinhardt et al., 2007, Protein Expr. & Purif. 54, 101-109), obtained from Millegen (Labege, France).

Erk2 (rat, recombinant) was assayed as described for CDK1 but using the specific substrate Ets1 (amino acids 1-138) in buffer A.

DYRK1A (rat, recombinant, expressed in E. coli as a GST fusion protein) was purified by affinity chromatography on glutathione-agarose and assayed as described for CDK1/cyclin B using myelin basic protein (1 mg/ml) as a substrate.

### Results

The results are given in Table 6, indicating that the enzymatic activity of Dyrk1A and Ckone is markedly reduced by the DLT1 compound and to a lesser extent reduced by the DLT5 compound.

TABLE 6
% ENZYMATIC ACTIVITY (10 μM 1% DMSO_{F})

| | CDK5 | GSK3 | pfGSK3 | Ckone | Dyrk1A | Erk2 | CDK2A |
|---|---|---|---|---|---|---|---|
| DLT1 | 87 | 77 | 92 | 62 | 49 | 99 | 91 |
| DLT5 | 95 | 81 | 89 | 73 | 74 | 92 | 100 |

DLT1 compound inhibit 51% of DYRK1A kinase activity at 10 μM

From examples 3-5 4 it follows that the DLT compounds tested in the present invention have a mechanism of action that acts through inhibition of kinases of the Aurora type or the Dyrk1A kinase all indeed known to be involved in cellular proliferation. This finding is of course helpful for designing screening assays for compounds having an anti-proliferative effect on cancer or tumour cells.

### Example 6

### Aurora A, B and C, WEE1 and DYRK-1A Kinase Inhibition Profile of DLT95, DLT95-F, DLT95-Cl and Vanillic Acid at 8 Different Concentrations

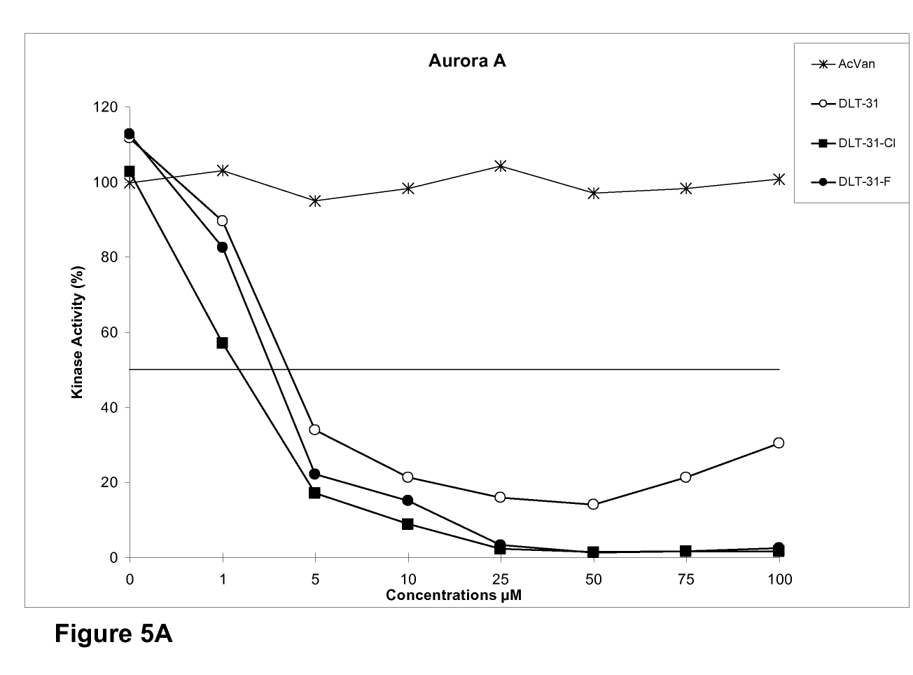

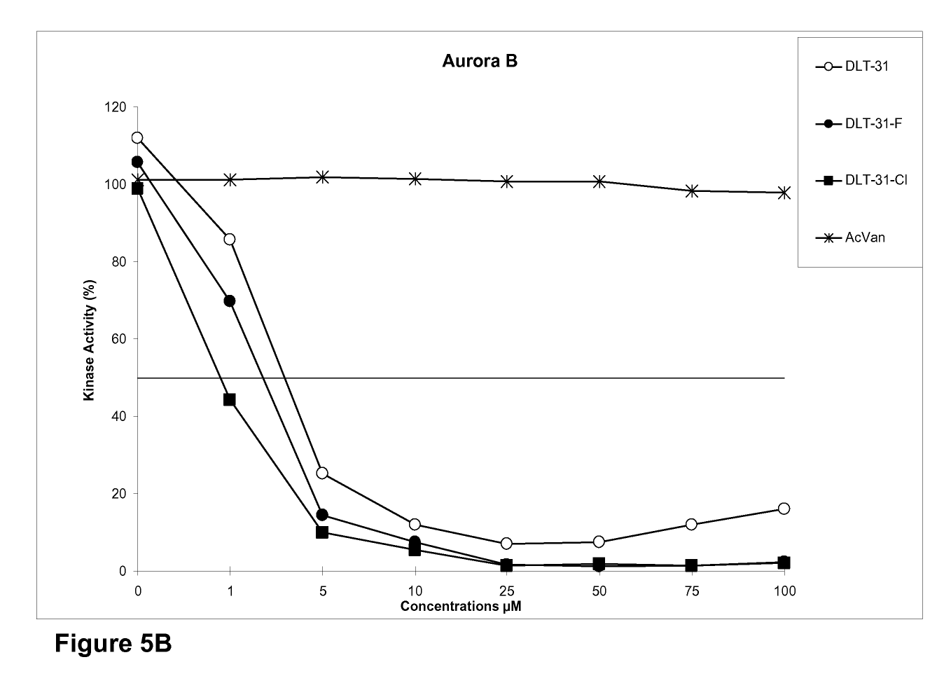

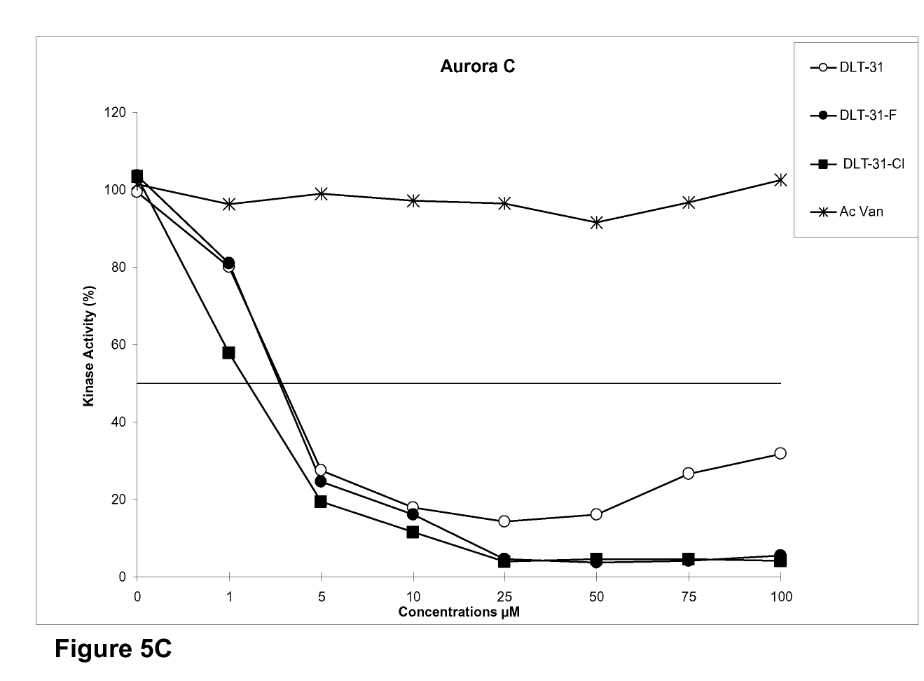

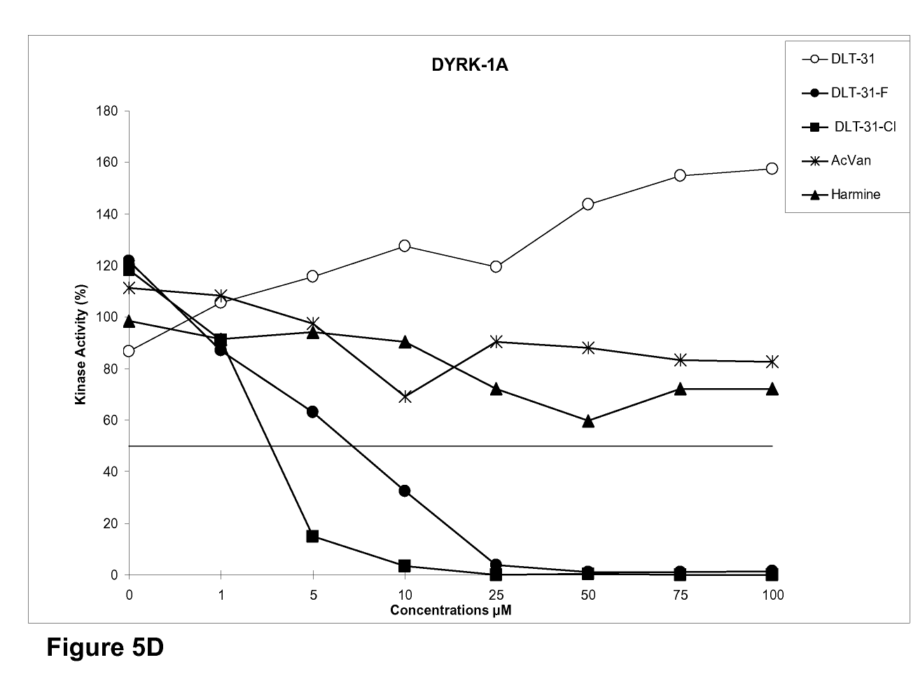

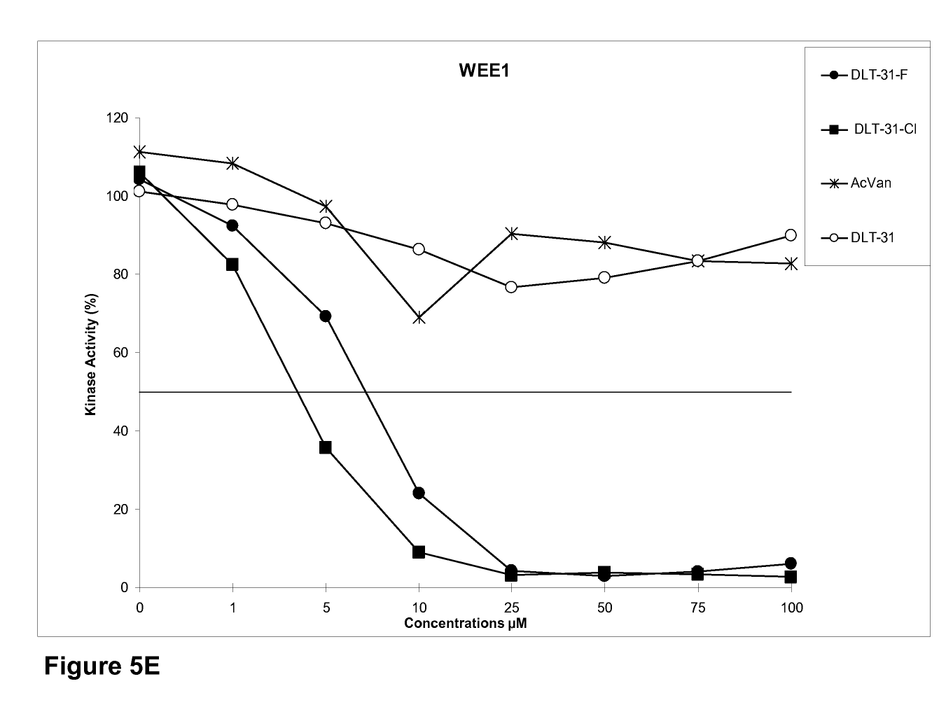

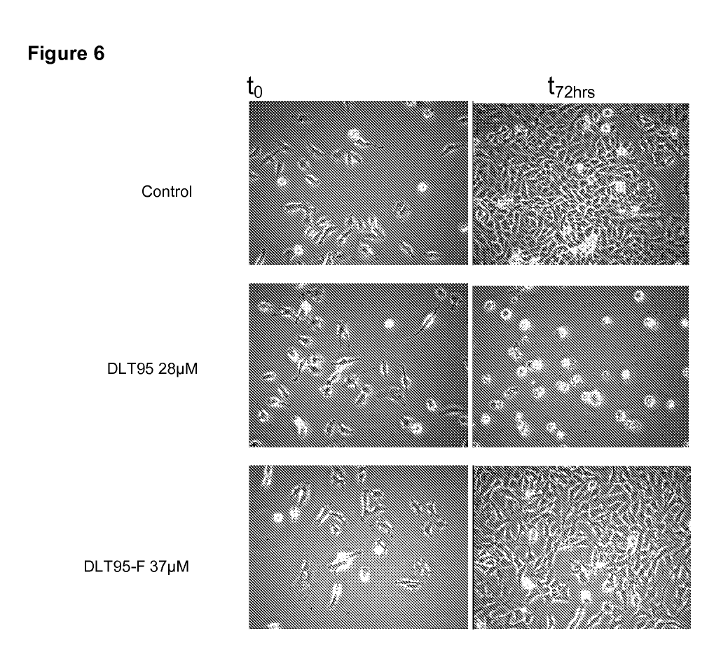

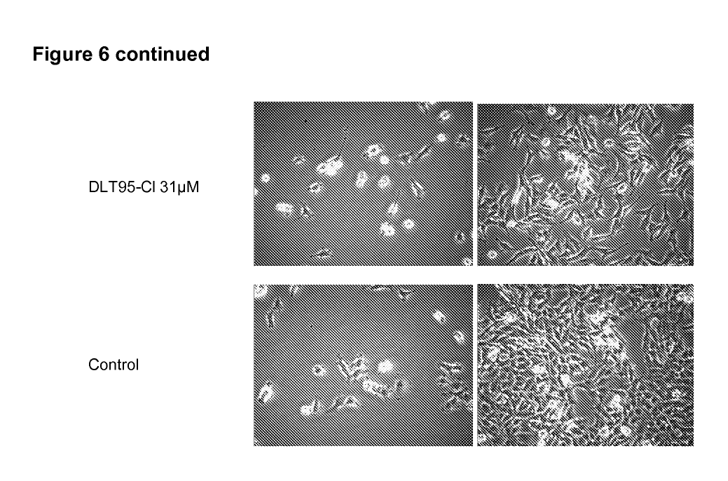

In addition, the inventors have embarked on a route to identify the actual targets of the compounds of the invention and have established that certain kinases, known to be involved in proliferation disorders are inhibited by some of the compounds of the invention. The results are presented in Table 7, FIG. 5 and example 7.

We established dose-response curves for the inhibition of Aurora A, B and C, WEE1 and DYRK1A kinases activity for the three DLT-95-compounds and vanillic acid at 0, 1, 5, 10, 25, 50, 75 and 100 μM with the same protocol as described below (example 7) except that we performed the experiments in triplicates (n=3). As can be seen from the figure, all 8 compounds have a marked effect on the Aurora kinases A, B and C, indicating a common target for the DLT compounds of the invention. DLT95-Cl compound is the most potent inhibitor for the 5 kinases with IC50≦3 μM.

TABLE 7
IC₅₀: Concentration of drug (μM) needed in order to
inhibit kinase activity by 50%

| Compounds | AurA | AurB | AurC | DYRK-1A | WEE1 |
|---|---|---|---|---|---|
| DLT-95-Cl | 1.7 | 0.9 | 1.8 | 3.2 | 3.74 |
| DLT-95-F | 3.2 | 2.4 | 4.7 | 7.1 | 7.12 |
| DLT-95 | 9.1 | 3.4 | 3.3 | >100 | >100 |
| AcVan | >100 | >100 | >100 | >100 | >100 |

### Example 7

### Kinase Inhibition Profile of DLT31-Cl at 20 μM

In view of the results in FIG. 5 and Table 7, the inventors performed a broader analysis on impairment of 255 protein kinases by DLT95-Cl compound of the invention.

255 kinases where tested with DLT95-Cl at 20 μM to establish the kinase inhibition profile of the compound.

The activity of 249 over the 255 kinases screened is ≧50% impaired by DLT95-Cl at 20 μM. In September 2009 we will perform the same experiment with DLT95-Cl at 2 μM to know if we maintain a potent kinase inhibition activity with ten time lower DLT95-Cl concentration.

### General Procedure to Test the Kinase Activity Inhibition of the Compounds Under Study:

The kinase inhibition profile of DLT95-Cl at 20 μM was determined using 255 protein kinases. Residual activity values were measured by testing each compound at one concentration in duplicate in each kinase assay. A radiometric protein kinase assay (33 PanQinase®Activity Assay) was used for measuring the kinase activity of 250 protein kinases. All kinase assays were performed in 96-well flashPlates™ from Perkin Elmer (Boston, Mass., USA) in a 50 μl reaction volume. The reaction cocktail was pipetted in 4 steps in the following order:
10 μl of non-radioactive ATP solution (in H₂O)
25 μl of assay buffer/[γ-33P]-ATP mixture
5 μl of test sample in 10% DMSO
10 μl of enzyme/substrate mixture

The assay for all enzymes contained 60 mM HEPES-NaOH, pH 7.5, 3 mM MgCl₂, 3 mM MnCl2, 3 μM Na-orthovanadate, 1.2 mM DTT, 50 μg/ml PEG20000, 1 μM ATP/[γ-33P]-ATP (approx. 6×1005 cpm per well), protein kinase, and substrate. All PKC assays (except the PKC-mu and the PKC-nu assay) additionally contained 1 mM CaCl2, 4 mM EDTA, 5 μg/ml Phosphatidylserine and 1 μg/ml 1,2-Dioleyl-glycerol. The MYLK2, CAMK1D, CAMK2A, CAMK2B, CAMK2D, CAMK4, CAMKK2, DAPK2 and EEF2K assays additionally contained 1 μg/ml Calmodulin and 0.5 mM CaCl2. The PRKG1 and PRKG2 assays additionally contained 1 μM cGMP.

### Recombinant Protein Kinases:

The protein kinases were expressed either in Sf9 insect cells or in E. coli as recombinant GST-fusion proteins or as His-tagged proteins. All kinases were produced from human cDNAs. Kinases were purified by affinity chromatography using either GSH-agarose (Sigma) or Ni-NTH-agarose (Qiagen). The purity of the protein kinases was examined by SDS-PAGE/coomassie staining. The identity of the protein kinases was checked by mass spectroscopy.

The reaction cocktails were incubated at 30° C. for 60 minutes. The reaction was stopped with 50 μl of 2% (v/v) H₃PO₄, plates were aspirated and washed two times with 200 μl 0.9% (w/v) NaCl. All assays were performed with a BeckmanCoulter Biomek 2000/SL robotic system.

Incorporation of ³³Pi (counting of “cpm”) was determined with a microplate scintillation counter (Microbeta, Wallac).

For each kinase, the median value of the cpm of three wells with complete reaction cocktails, but without kinase, was defined as “low control” (n=2). This value reflects unspecific binding of radioactivity to the plate in the absence of protein kinase but in the presence of the substrate. Additionally, for each kinase the median value of the cpm of three other wells with the complete reaction cocktail, but without any compound, was taken as the “high control”, i.e. full activity in the absence of any inhibitor (n=2). The difference between high and low control of was taken as 100% activity for each kinase. As part of the data evaluation the low control value of each kinase was subtracted from the high control value as well as from their corresponding “compound values”. The residual activity (in %) for each compound well was calculated by using the following formula: Res. Activity (%)=100×[(cpm of compound−low control)/(high control−low control)]

The data obtained reveal that the compounds under study are potent pan-antikinase inhibitors, with marked inhibition activity observed for compound DLT-95-Cl. The large set of kinases targeted by compound DLT-95-Cl are overexpressed in most of those cancers associated with dismal prognoses, i.e. any cancer prone to metastasize (keeping in mind that >90% of cancer patients die from their metastases. Cancers that do not metastasize, such as malignant gliomas, also overexpress the kinases targeted by compound DLT-95-Cl. Compound DLT-95-Cl could therefore be used to combat alone or in combination with other treatments (including for example radiotherapy and chemotherapy) those cancers which are prone to metastasize and/or which already metastasized and/or malignant gliomas.

1. A compound of general formula (I): 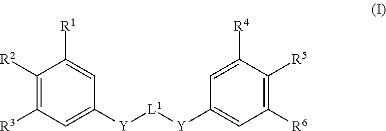
wherein each of R¹⁻⁶, independently of each other is H, OH, C₁₋₈ alkoxyalkylene, OMe, Ac, OAc, C₁₋₈ alkyl, NO₂ or a halogen, and wherein two contiguous substituents among R¹⁻³ may together form a dioxole;
wherein Y is selected from the group consisting of COO, tetrazole, OCO, OCOO, CONR¹⁰, NR¹⁰CO, OCONR¹⁰, NR¹⁰COO, NR¹⁰CONR¹⁰, COCH₂CO, COCH₂CH₂, CH₂CH₂CO, CH₂COCH₂, COOCH₂, CONHCH₂, CON—C₁₋₆alkylCH₂, CONHCO, CON—C₁₋₆alkylCO, CH₂NHCH₂, CH₂N—C₁₋₆alkylCH₂, CH₂OCO, CH₂NHCO, CH₂N(C₁₋₆alkyl)CO, CH₂OCH₂, CH₂SCH₂, SO₂OCH₂, SO₂NHCH₂, and SO₂N—C₁₋₆alkylCH₂, wherein R¹⁰=H or C₁₋₄ Alkyl, wherein R¹¹=C₁₋₆alkyl or CH2;
wherein L¹ is
C₁₋₈alkylene;
(CH₂)ₙ, wherein n is 2-10; 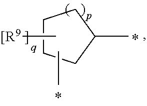
wherein
R⁹ is selected from the group consisting of OH, CO₂H, and NH₂;
q is 0, 1, 2, or 3, each group being unsubstituted or
substituted with one, two or three substituents each independently selected from the group consisting of C₁₋₆alkyl, CO₂H, vanillic acid, amine, and C₁₋₆alkyloxycarbonyl; and
p is an integer from 0, 1, 2, or 3; or 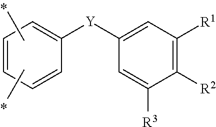
or stereoisomeric forms, pharmaceutically acceptable addition salts, hydrates or solvates thereof.

2. The compound according to claim 1, having general formula (VI): 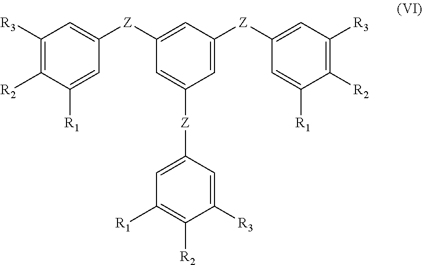
wherein Z is selected from the group consisting of COCH₂CO, COCH₂CH₂, CH₂CH₂CO, CH₂COCH₂, COOCH₂, CONHCH₂, CON—C₁₋₆alkylCH₂, CONHCO, CON—C₁₋₆alkylCO, CH₂NHCH₂, CH₂N—C₁₋₆alkylCH₂, CH₂OCO, CH₂NHCO, CH₂N(C₁₋₆alkyl)CO, CH₂OCH₂, CH₂SCH₂, SO₂OCH₂, SO₂NHCH₂, and SO₂N—C₁₋₆alkylCH₂;
and wherein each of R¹⁻³, independently of each other, is H, OH, Halogen, C₁₋₈ alkoxyalkylene, OMe Ac, OAc, C₁₋₈ alkyl, or NO₂; and wherein two contiguous substituents among R¹⁻³ can be may together form a dioxole.

3. The compound according to claim 2, selected from the group consisting of: [3,5-bis-[(4-hydroxy-3-methoxy-benzoyl)-oxymethyl]-phenyl]-methyl-4-hydroxy-3-methoxy-benzoate (DLT95), [3,5-bis [(4-hydroxy-3-fluoro-benzoyl)oxymethyl]-phenyl]-methyl-4-hydroxy-3-fluoro-benzoate (DLT95-F), and [3,5-bis-[(4-hydroxy-3-chloro-benzoyl)-oxymethyl]-phenyl]-methyl-4-hydroxy-3-chloro-benzoate (DLT95-Cl).

4. The compound according to claim 1, defined by the general formula (II): 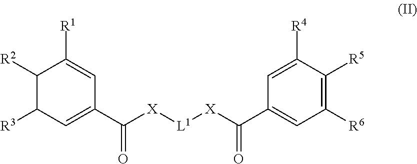
wherein X is selected from the group consisting of O, O—C₁₋₆alkyl, NH, and N—C₁₋₆alkyl;
wherein each of R¹, R², R³, R⁴, R⁵, R⁶ is independently selected from the group consisting of H, OH, C₁₋₈alkoxyC₁₋₆alkyl, C₁₋₆alkoxy, and halogen;
L¹ is selected from the group consisting of
C₁₋₈alkylene; 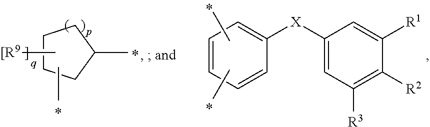
wherein the asterisk is used herein to indicate the point at which a mono- or bivalent radical depicted is connected to the structure to which it relates and of which the radical forms part;
each group being unsubstituted or substituted with one, two or three substituents each independently selected form the group consisting of C₁₋₆alkyl, CO₂H, vanillic acid, amine, and C₁₋₆alkyloxycarbonyl, wherein p is an integer selected from 0, 1, 2, or 3;
R⁹ is selected from the group consisting of OH, CO₂H, and NH₂ and q is 0, 1, 2, or 3;
or stereoisomeric forms, pharmaceutically acceptable addition salts, hydrates or solvates thereof.

5. The compound according to claim 1, wherein R¹ and R⁴ are each independently hydrogen, and R², R³, R⁵ and R⁶ are each independently hydrogen, hydroxyl, or C₁₋₆alkoxy.

6. The compound according to claim 1, wherein R¹ and R⁴ are each independently hydrogen, and R², R³, R⁵ and R⁶ are each independently hydroxyl or C₁₋₆alkoxy.

7. The compound according to claim 1, wherein R¹ and R⁴ are each independently hydrogen, R² and R⁵ are each independently C₁₋₆alkoxyl and R³ and R⁶ are each independently hydroxyl.

8. The compound according to claim 1, wherein R¹ and R⁴ are each independently hydrogen, R² and R⁵ are each independently methoxy and R³ and R⁶ are each independently hydroxyl.

9. The compound according to claim 1, wherein X is oxygen.

10. The compound according to claim 1, wherein X is NH.

11. The compound according to claim 4, wherein X═OCH2, L¹ is 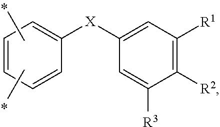
wherein the asterisk is used herein to indicate the point at which a mono- or bivalent radical depicted is connected to the structure to which it relates and of which the radical forms part, and R¹ and R⁴=OMe, F, or Cl, R² and R⁵=OH, and R³ and R⁶=H

12. The compound according to claim 1, having formula (III) 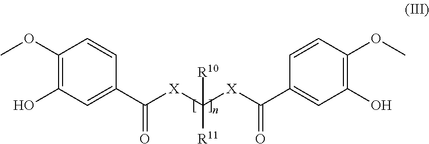
wherein X is selected from the group consisting of O, O—C₁₋₆alkyl, NH, and N—C₁₋₆alkyl;
wherein n is 1, 2, 3, 4, 5, 6, 7, or 8; and
wherein R¹⁰ and R¹¹ are, each independently selected from the group comprising consisting of H, CO₂H, or C₁₋₆alkyl and vanillic acid.

13. The compound of claim 12, wherein X is oxygen, R¹⁰ and R¹¹ are hydrogen, and n is 2.

14. The compound of claim 12, wherein X is NH R¹⁰ and R¹¹ are hydrogen, and n is 2.

15. The compound of claim 13, selected from the group consisting of Ethane-1,2-diyl bis-(4-hydroxy-3-methoxybenzoate), Propane-1,3-diyl bis-(4-hydroxy-3-methoxybenzoate), Butane-1,4-diyl bis-(4-hydroxy-3-methoxybenzoate), Pentane-1,5-diyl bis-(4-hydroxy-3-methoxybenzoate), and Hexane-1,6-diyl bis-(4-hydroxy-3-methoxybenzoate).

16. The compound according to claim 1, having formula (IV) 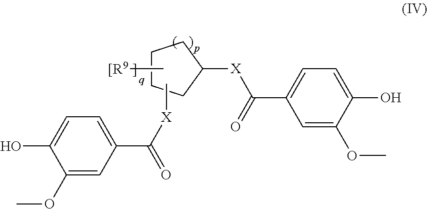
wherein X is selected from the group consisting of O, O—C₁₋₆alkyl, NH, and N—C₁₋₆alkyl;
wherein p is 0, 1, 2, or 3; and
R⁹ is selected from the group consisting of OH, CO₂H, and NH_{z} and q is 0, 1, 2, or 3.

17. The compound of claim 14, wherein X is oxygen, p is 2, and q is 0.

18. The compound of claim 14, wherein X is NH, p is 2, and q is 0.

19. The compound according to claim 17, selected from the group consisting of trans-cyclohexane-1,2-diyl bis-(4-hydroxy-3-methoxybenzoate); cis-cyclohexane-1,2-diyl bis-(4-hydroxy-3-methoxybenzoate); racemic cyclohexane-1,3-diyl bis-(4-hydroxy-3-methoxybenzoate); cis-cyclohexane-1,3-diyl bis-(4-hydroxy-3-methoxybenzoate); trans-cyclohexane-1,3-diyl bis-(4-hydroxy-3-methoxybenzoate); cis-cyclohexane-1,4-diyl bis-(4-hydroxy-3-methoxybenzoate); trans-cyclohexane-1,4-diyl bis-(4-hydroxy-3-methoxybenzoate); and racemic cyclohexane-1,4-diyl bis-(4-hydroxy-3-methoxybenzoate).

20. The compound of claim 19, wherein the compound is trans-cyclohexane-1,2-diyl bis-(4-hydroxy-3-methoxybenzoate).

21. The compound according to claim 1, selected from the group consisting of: [2-[(4-hydroxy-3-methoxy-benzoyl)oxymethyl]phenyl]methyl 4-hydroxy-3-methoxy-benzoate, 6-(3,4-dimethoxybenzoyl)oxyhexyl 3,4-dimethoxybenzoate, 1,4-oxybut-2-enyl-bis(4-hydroxy-3-methoxybenzoate), 1,4-oxybut-2-ynyl bis(4-hydroxy-3-methoxybenzoate), 6-(3-hydroxy-4-methoxy-benzoyl)oxyhexyl 3-hydroxy-4-methoxy-benzoate, [3-[(4-hydroxy-3-methoxy-benzoyl)oxymethyl]phenyl]methyl-4-hydroxy-3-methoxy-benzoate, 2-[bis[2-(4-hydroxy-3-methoxy-benzoyl)oxyethyl]amino]ethyl-4-hydroxy-3-methoxy-benzoate, and [7-[(4-hydroxy-3-methoxy-benzoyl)oxymethyl]-2,6-dimethyl-3,5-dioxo-pyrazolo[1,2-a]pyrazol-1-yl]methyl-4-hydroxy-3-methoxy-benzoate.

22. The compound according to claim 1, having formula (V) 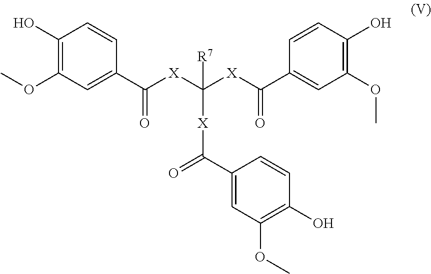
wherein X is selected from the group consisting of O, O—C₁₋₆alkyl, NH, and N—C₁₋₆alkyl;
wherein R⁷ is H, CO₂H, or C₁₋₆alkyl.

23. A compound having formula (IIa) 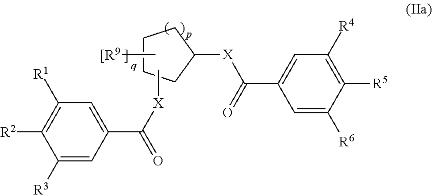
wherein X is selected from the group consisting of O, O—C₁₋₆alkyl, NH, and N—C₁₋₆alkyl;
wherein each R¹, R², R³, R⁴, R⁵, R⁶ is independently selected from the group consisting of H, OH, C₁₋₈alkoxyC₁₋₆alkyl, C₁₋₆alkoxy, and halogen; each group being optionally substituted with one, two or three substituents each independently selected form the group consisting of amide, amine, C₁₋₆alkyl, CO₂H, vanillic acid, and C₁₋₆alkyloxycarbonyl;
wherein p is 0, 1, 2, or 3; and
wherein R⁹ is selected from the group consisting of OH, CO₂H, and NH₂ and q is 0, 1, 2, or 3.

24. A pharmaceutical composition comprising the compound of claim 1 and a pharmaceutically acceptable carrier.

25-26. (canceled)

27. A method of treating a proliferative disorder in a subject in need thereof, the method comprising administering a therapeutically effective amount of the compound according to claim 1 to the subject.

28. A method of treating an oxidative or inflammatory disorder in a subject needing such therapy in need thereof, the method comprising administering a therapeutically effective amount of the compound according to claim 1 to the subject.

29. The method of according to claim 28, further comprising treating the subject with a therapy selected from the group consisting of: chemotherapy, radiation therapy, immunotherapy, gene therapy, and any combination thereof.

30. The method according to claim 27, further comprising treating the subject with one or more active compounds, before, after or simultaneously with the administration of the compound of claim 1.

31. The compound of claim 1, wherein the composition is comprised in pills, tablets, lacquered tablets, sugar-coated tablets, granules, hard and soft gelatin capsules, aqueous, alcoholic or oily solutions, syrups, emulsions, suspensions, suppositories, solutions for injection or infusion, ointments, tinctures, sprays or transdermal therapeutic systems, nasal sprays or aerosol mixtures, microcapsules, implants, or rods.

32. A method for identifying agents for treating proliferative disorders, Alzheimer's disease, Parkinson's disease or Pick's disease or for ameliorating symptoms of Down syndrome that inhibit the activity of one or more of the kinases selected from the group of: Aurora A, B, or C kinase, and DYRK1A kinase; the method comprising measuring the activity of said one or more kinases in the presence and absence of said agent, wherein a decrease in enzyme activity in the presence of the agent indicates that it is an inhibitory agent.

33. The method according to claim 32, wherein the agent is a compound of claim 1.

34. A method for treating a proliferative disorder, Alzheimer's disease, Parkinson's disease or Pick's disease or for ameliorating symptoms of Down syndrome in a subject, the method comprising administering a therapeutically effective amount of the compound according to claim 1 to the subject.

35. The method according to claim 28, further comprising treating the subject with one or more active compounds, before, after or simultaneously with the administration of the compound of claim 1.


SEQUENCE LISTING (2 sequences)

SEQ ID NO: 1 - protein, 26 aa, Artificial
  Substrate for GSK-3 enzyme
YRRAAVPPSPSLSRHSSPHQSEDEEE

SEQ ID NO: 2 - protein, 15 aa, Artificial
  Substrate for CK-1 enzyme
RRKHAAIGSAYSITA